(19) United States
(12) Patent Application Publication
(10) Pub. No.: US 2009/0105477 A1
(43) Pub. Date: Apr. 23, 2009

(54) Quinazoline Protein Tyrosine Phosphatase Inhibitors

(76) Inventors: Steven Joseph Berthel, Mendham Township, NJ (US); Adrian Wai-Hing Cheung, Glen Rock, NJ (US); Kshitij Chhabilbhai Thakkar, Clifton, NJ (US); Weiya Yun, Warren, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 12/261,138

(22) Filed: Oct. 30, 2008

Related U.S. Application Data
(63) Continuation of application No. 11/268,300, filed on Nov. 6, 2005.
(60) Provisional application No. 60/715,260, filed on Sep. 8, 2005.
(60) Provisional application No. 60/626,288, filed on Nov. 9, 2004.

(51) Int. Cl. C07D 239/72

(52) U.S. Cl. 544/291

(57) ABSTRACT

The present invention comprises aminoquinazoline compounds of the general formula I:

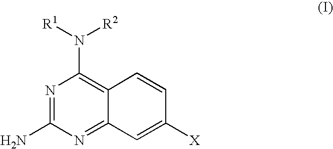

wherein X is an unsubstituted or substituted phenyl, or is an unsubstituted or substituted 5 or 6 membered heteroaromatic ring. The compounds of the present invention are potent inhibitors of PTP1B. Accordingly, the invention also encompasses pharmaceutical compositions and methods of treating or preventing PTP-1B mediated diseases, including diabetes, obesity, and diabetes-related diseases.

### PRIORITY TO RELATED APPLICATION

This application is a continuation of U.S. application Ser. No. 11/268,300 filed Nov. 6, 2005, which claims the benefit of U.S. Provisional Application No. 60/715,260, filed Sep. 8, 2005, and U.S. Provisional Application No. 60/626,288, filed Nov. 9, 2004. The entire contents of the above-identified applications are hereby incorporated by reference.

### BACKGROUND OF THE INVENTION

Protein tyrosine phosphatases (PTPases) are key enzymes in processes that regulate cell growth and differentiation. The inhibition of these enzymes can play a role in the modulation of multiple signaling pathways in which tyrosine phosphorylation dephosphorylation plays a role. PTP1B is a particular protein tyrosine phosphatase that is often used as a prototypical member of that class of enzymes. Kennedy et al., 1999, Science 283: 1544-1548 showed that protein tyrosine phosphatase PTP-1B is a negative regulator of the insulin signaling pathway, suggesting that inhibitors of this enzyme may be beneficial in the treatment of diabetes.

PTPase inhibitors are recognized as potential therapeutic agents for the treatment of diabetes. See, e.g. Moeller et al., 3(5):527-40, Current Opinion in Drug Discovery and Development, 2000; or Zhang, Zhong-Yin, 5:416-23, Current Opinion in Chemical Biology, 2001. The utility of PTPase inhibitors as therapeutic agents has been a topic of discussion in several review articles, including, for example, Expert Opin Investig Drugs 12(2):223-33, February 2003.

Inhibitors of PTP-1B have utility in controlling or treating Type 1 and Type 2 diabetes, in improving glucose tolerance, and in improving insulin sensitivity in patients in need thereof.

### SUMMARY OF THE INVENTION

The present invention comprises aminoquinazoline compounds of the general formula I:

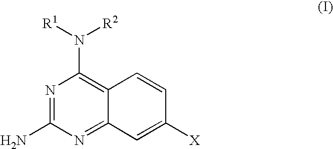

wherein X is an unsubstituted or substituted phenyl, or is an unsubstituted or substituted 5 or 6 membered heteroaromatic ring. The compounds of the present invention are potent inhibitors of PTP1B. Accordingly, the invention also encompasses pharmaceutical compositions and methods of treating or preventing PTP-1B mediated diseases, including diabetes, obesity, and diabetes-related diseases.

### DETAILED DESCRIPTION OF THE INVENTION

The present invention comprises compounds of the formula I:

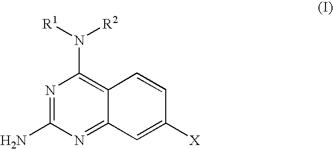

wherein X is a group X-1 of the formula:

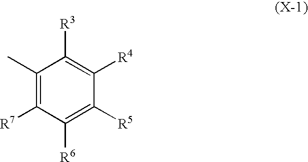

or X is a group X-2 of the formula:

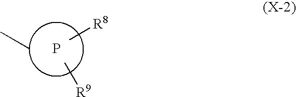

wherein
R¹ and R² are independently selected from the group consisting of hydrogen, lower alkyl, alkoxy lower alkyl, hydroxy lower alkyl, except that R¹ and R² may not both be hydrogen. It is preferred that the lower alkyl, alkoxy lower alkyl, and hydroxy lower alkyl groups have up to 4 carbon atoms with C1-4 alkyl and hydroxy C1-3 alkyl being more preferred; and it is most preferable that one of R¹ or R² is hydrogen.

R³, R⁴, R⁶ and R⁷ are each independently selected from the group consisting of hydrogen, lower alkyl, substituted lower alkyl, lower alkoxy, substituted lower alkoxy, hydroxy, halogen, lower alkylthio, lower alkylsulfinyl, lower alkylsulfonyl, aminosulfonyl, cyano, nitro, carbamoyl, lower alkylcarbamoyl, lower alkanoyl, aroyl, aryl, aryloxy, aryl lower alkoxy, aryl loweralkenyl, aryl lower alkynyl, lower alkenyl, lower alkynyl, lower alkylamino, substituted lower alkylamino, lower alkanoylamino, sulfonylamino, cycloalkyl, heterocycloalkyl, heterocyclyloxy, heterocyclylcarbonyl, carboxyl, lower alkoxy carbonyl, and a substituent of the formula:

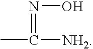

Preferred substituents for R³ and R⁷ are halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl. Chlorine, fluorine, trifluoromethyl, C1-4 alkyl, C1-3 alkylthio, C1-3 alkylsulfonyl, C1-3 alkoxy, C1-3 alkoxy substituted with a group selected from hydroxy, methoxy and ethoxy are still more preferred.

Preferred substituents for R⁴ and R⁶ are hydrogen, halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl. Hydrogen, chlorine, fluorine, trifluoromethyl, C1-4 alkyl, C1-3 alkylthio, C1-3 alkylsulfonyl, C1-3 alkoxy, C1-3 alkoxy substituted with a group selected from hydroxy, methoxy and ethoxy are further preferred. Hydrogen is more preferred.

R⁵ is selected from the group consisting of hydrogen, lower alkyl, lower alkoxy, alkoxy lower alkyl, alkoxy lower alkoxy, hydroxy lower alkyl, hydroxy, hydroxyalkoxy, halogen, lower alkylthio, lower alkylsulfinyl, lower alkylsulfonyl, perfluoro lower alkyl, alkanyoyl, aroyl, aryl alkynyl, lower alkynyl and lower alkanoylamino. Hydrogen is preferred.

\textcircled{P} is a 5 or 6 membered heteroaromatic ring containing from 1 to 2 hetero atoms selected from the group consisting of oxygen, sulfur and nitrogen;

R⁸ and R⁹ are each independently selected from the group consisting of hydrogen, lower alkyl, lower alkoxy, perfluoro lower alkyl, halogen, aryl lower alkyl, aryl, and aryl lower alkoxy.

As used in this specification, the term “lower alkyl”, alone or in combination (for example, as part of “lower alkoxy,” “lower alkanoyl,” “lower alkylamino,” etc. defined below), means a straight-chain or branched-chain alkyl group containing a maximum of six carbon atoms, such as methyl, ethyl, n-propyl, isopropyl, n-butyl, sec. butyl, isobutyl, tert.butyl, n-pentyl, n-hexyl and the like. “Substituted” in front of “lower alkyl” or a lower alkyl combination such as “lower alkoxy,” “lower alkanoyl”, “lower alkylamino,” etc., means the lower alkyl portion is substituted by one or more groups selected independently from cycloalkyl, heterocycloalkyl, nitro, aryloxy, aryl, heteroaryl, hydroxy, halogen, cyano, lower alkoxy, lower alkoxycarbonyl, lower alkanoyl, lower alkylthio, lower alkyl sulfinyl, lower alkyl sulfonyl, and substituted amino, e.g., dimethylamino. Preferred substituents are hydroxy, halogen, nitro, lower alkoxy, phenoxy, phenyl and lower alkylthio. Examples of substituted lower alkyl groups include 2-hydroxyethyl, 2-methoxypropyl, 3-oxobutyl, cyanomethyl, trifluoromethyl, 2-nitropropyl, benzyl, including p-chloro-benzyl and p-methoxy-benzyl, and 2-phenyl ethyl. The term “hydroxy lower alkyl” means a lower alkyl group which is mono- or di-substituted with hydroxy. The term “alkoxy lower alkyl” means a lower alkyl group mono-substituted with a lower alkoxy.

The term “lower alkoxy carbonyl” means a carboxyl group whose hydrogen is substituted with lower alkyl.

The term “lower alkoxy” means a lower alkyl group bonded through an oxygen atom. Examples of unsubstituted lower alkoxy groups are methoxy, ethoxy, n-propoxy, isopropoxy, n-butoxy, tert-butoxy and the like. “Alkoxy lower alkoxy” means a lower alkoxy group substituted with a C₁₋₃ alkoxy. “Hydroxy lower alkoxy” means a lower alkoxy group which is mono- or disubstituted with hydroxy.

The term “lower alkylthio” means a lower alkyl group bonded through a divalent sulfur atom, for example, a methyl mercapto or a isopropyl mercapto group. The term “lower alkylsulfinyl” means a lower alkyl group as defined above bound to the rest of the molecule through the sulfur atom in the sulfinyl group. The term “lower alkylsulfonyl” means a lower alkyl group as defined above bound to the rest of the molecule through the sulfur atom in the sulfonyl group.

The term “lower alkanoyl” means lower alkyl groups bonded to the rest of the molecule via a carbonyl group and embraces in the sense of the foregoing definition groups such as formyl (methanoyl), acetyl, propionyl and the like. The term “perfluoro lower alkanoyl” means a perfluoro lower alkyl group which is bonded to the rest of the molecule via a carbonyl group. “Lower alkanoylamino” means a lower alkanoyl group bonded to the rest of the molecule via an amino group. “Lower alkylamino” means a lower alkyl group bonded to the rest of the molecule via an amino group.

The term “carbamoyl” means the carboxamide substituent —C(O)—NH₂. The term “lower alkylcarbamoyl” means that one or both hydrogen atoms of the amide are independently substituted with lower alkyl.

The term “cycloalkyl” means an unsubstituted or substituted 3- to 6-membered carbocyclic ring. Substituents useful in accordance with the present invention are hydroxy, halogen, cyano, lower alkoxy, lower alkanoyl, lower alkyl, substituted lower alkyl, aroyl, lower alkylthio, lower alkyl sulfinyl, lower alkyl sulfonyl, aryl, heteroaryl and substituted amino. Preferred substitutents are hydroxy, halogen, lower alkoxy, lower alkyl, phenyl and benzyl.

The term “heterocycloalkyl” means an unsubstituted or substituted 5- to 6-membered carbocyclic ring in which one or two of the carbon atoms has been replaced by heteroatoms independently selected from O, S and N. “Heterocyclyl carbonyl” means a heterocycloalkyl group which is bonded to the rest of the molecule via a carbonyl group. “Heterocyclyloxy” means a heterocycloalkyl group which is bonded via an oxygen atom. Preferred heterocycloalkyl groups are pyrrolidinyl and morpholinyl. Substituents useful in accordance with the present invention are hydroxy, halogen, cyano, lower alkoxy, lower alkanoyl, lower alkyl, substituted lower alkyl, aroyl, lower alkylthio, lower alkylsulfinyl, lower alkylsulfonyl, aryl, heteroaryl and substituted amino. Preferred substitutents useful in accordance with the present invention are hydroxy, halogen, lower alkoxy, lower alkyl and benzyl.

The term “aryl” means a monocylic aromatic group, such as phenyl, which is unsubstituted or substituted by one to three conventional substituent groups selected from lower alkyl, lower alkoxy, hydroxy lower alkyl, hydroxy, hydroxyalkoxy, halogen, lower alkylthio, lower alkylsulfinyl, lower alkylsulfonyl, cyano, nitro, perfluoro lower alkyl, alkanoyl, aroyl, aryl alkynyl, heteroaryl, lower alkynyl and lower alkanoylamino. Examples of aryl groups that may be used in accordance with this invention are unsubstituted phenyl, m- or o-nitrophenyl, p-tolyl, m- or p-methoxyphenyl, 3,4-dimethoxyphenyl, p-chlorophenyl, p-cyanophenyl, m-methylthiophenyl, 2-methyl-5-nitrophenyl, 2,6-dichlorophenyl, m-perfluorophenyl, and the like.

The term “aryloxy” means an aryl group, as hereinbefore defined which is bonded via an oxygen atom. The preferred aryloxy group is phenoxy.

The term “lower alkenyl” means an alkene group having from 2 to 6 carbon atoms with a double bond located between any two adjacent carbon atoms.

The term “lower alkynyl” means an alkyne group having from 2 to 6 carbon atoms with a triple bond located between any two adjacent carbon atoms.

The term “heteroaryl” means an unsubstituted or substituted 5- or 6-membered monocyclic hetereoaromatic ring containing one to three hetereoatoms which are independently N, S or O. Examples are pyridyl, thienyl, pyrimidinyl, oxazolyl, and furyl. Substituents as defined above for “aryl” are included in the definition of heteroaryl.

The term “perfluoro lower alkyl” means a lower alkyl group wherein all the hydrogens of the lower alkyl group are replaced by fluorine. Preferred perfluoro lower alkyl groups are trifluoromethyl and pentafluoroethyl.

The term “aminosulfonyl” means an amino group bound to the rest of the molecule through the sulfur atom of a sulfonyl group wherein the amino may be optionally further mono- or di-substituted with methyl or ethyl.

The term “sulfonylamino” means a sulfonyl group bound to the rest of the molecule through the nitrogen atom of an amino group wherein the sulfonyl group may be optionally further substituted with methyl or ethyl.

The term “aroyl” means an aryl or heteroaryl group as defined bonded to the rest of the molecule via a carbonyl group. Examples of aroyl groups are benzoyl, 3-cyanobenzoyl, and the like.

The term “aryl lower alkoxy” means a lower alkoxy group in which one hydrogen atom is replaced by an aryl group. Benzyloxy is preferred.

The term “pharmaceutically acceptable salts” refers to conventional acid-addition salts or base-addition salts that retain the biological effectiveness and properties of the compounds of formulas I, I-A and I-B, and are formed from suitable non-toxic organic or inorganic acids, or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as for example, tetramethylammonium hydroxide. The chemical modification of a pharmaceutical compound (i.e., drug) into a salt is a technique well known to pharmaceutical chemists to obtain improved physical and chemical stability, hygroscopicity, flowability and solubility of compounds. See, e.g., H. Ansel et al., Pharmaceutical Dosage Forms and Drug Delivery Systems (6th Ed. 1995) at pp. 196 and 1456-1457.

Intravenous, intramuscular, oral or inhalation administrations are preferred forms of use. The dosages in which the compounds of the invention are administered in effective amount depend on the nature of the specific active ingredient, the age and requirements of the patient and the mode of administration. Dosages may be determined by any conventional means, e.g., by dose-limiting clinical trials. In general, dosages of about 0.1 to 20 mg/kg body weight per day are preferred, with dosages of 0.5-10 mg/kg per day being particularly preferred.

The invention further comprises pharmaceutical compositions that contain a pharmaceutically effective amount of a compound of the invention and a pharmaceutically acceptable carrier. Such compositions may be formulated by any conventional means. Tablets or granulates can contain a series of binders, fillers, carriers or diluents. Liquid compositions can be, for example, in the form of a sterile water-miscible solution. Capsules can contain a filler or thickener in addition to the active ingredient. Furthermore, flavor-improving additives as well as substances usually used as preserving, stabilizing, moisture-retaining and emulsifying agents as well as salts for varying the osmotic pressure, buffers and other additives can also be present.

The previously mentioned carrier materials and diluents can comprise any conventional pharmaceutically acceptable organic or inorganic substances, e.g., water, gelatine, lactose, starch, magnesium stearate, talc, gum arabic, polyalkylene glycols and the like.

Oral unit dosage forms, such as tablets and capsules, preferably contain from 1 mg to 250 mg of a compound of this invention. The compounds of the invention may be prepared by conventional means.

In accordance with this invention, the compounds herein as well as their pharmaceutically acceptable salts are useful in the control or prevention of illnesses associated with high blood glucose concentration. A preferred indication associated with the present invention is that associated with diabetes.

The dosage can vary within wide limits and will, of course, have to be adjusted to the individual requirements in each particular case. In the case of oral administration, the dosage for adults may vary from about 1 mg to about 1000 mg per day of a compound of formula I, or of the corresponding amount of a pharmaceutically acceptable salt thereof. The daily dosage may be administered as single dose or in divided doses, and in addition, the upper limit can also be exceeded when this is found to be indicated.

The methods for preparing the compounds of this invention are described in the following schemes. In all schemes and examples herein it is understood that all carbon and heteroatoms, whether structurally depicted by letter or bond line drawing, are accompanied by the appropriate number of hydrogen atoms to complete the valence as appropriate.

SCHEME 1 shows the preparation of 7-bromo-quinazoline-2,4-diamine according to the procedure described by Hynes, J; Tomazici, A; Parrish, C; Fetzer, O. Journal of Heterocyclic Chemistry (1991), 28(5), 1357-63.

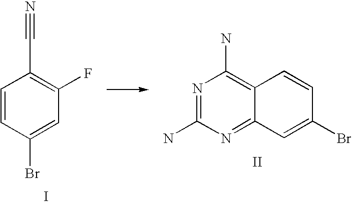

Compound II: A mixture of 4-bromo-2-fluorobenzonitrile I (5.24 g, 26.2 mmol) and guanidine carbonate (9.45 g, 52.5 mmol) in N,N-Dimethylacetamide (150 mL) were heated under nitrogen at 150-160° C. for 24 hours. The resultant reaction mixture was cooled, concentrated in vacuo, to get yellow solid. Water (150 mL) was added to the yellow solid followed by aqueous ammonium hydroxide (5 mL) till pH was 8-9. This resulted into very fine yellow solid which was washed with water followed by ether and then n-pentane to give 7-bromo-quinazoline-2,4-diamine II (6.18 g, 98.7%) as a pale yellow solid. ¹H NMR (DMSO-d₆, 400 MHz) δ 7.89 (d, J=8.79 Hz, 1H), 7.38 (broad s, 2H), 7.33 (d, J=1.95 Hz, 1H), 7.13 (dd, J₁=1.95 Hz, J₂=8.79 Hz, 1H), 6.18 (broad s, 2H).

Scheme 2 below provides a general synthesis step, and the examples provide a detailed description of the schematic methods:

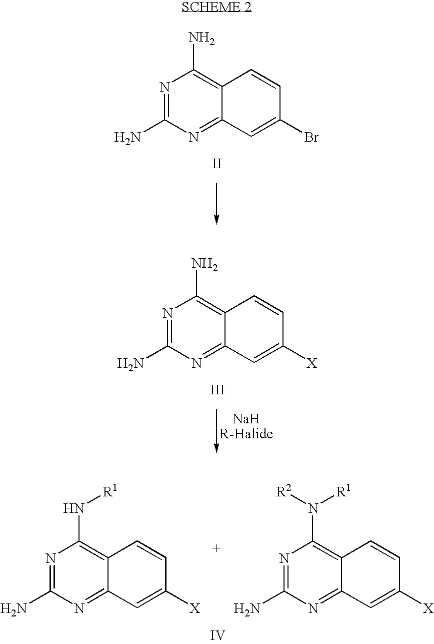

Compound III: The coupling reaction can be carried out by a conventional aryl coupling method, e.g., Suzuki coupling method: (a) Suzuki et al., synth. commun. 1981, 11, 513, (b) Suzuki pure and Appl. Chem. 1985, 57, 1749-1758, (c) Suzuki et al., Chem. Rev. 1995, 95, 2457-2483, (d) Shieh et al., J. Org. Chem. 1992, 57, 379-381, (e) Martin et al., Acta Chemica Scandinavica. 1993, 47, 513.

Typical conditions used to carry out the Suzuki coupling of 7-bromo-quinazoline-2,4-diamine II includes the use of either commercially appropriate aryl or heteroaromatic boronic acid or esters (e.g. where Ar is defined as aryl) as coupling partner, in aqueous base such as sodium bicarbonate or potassium carbonate or barium hydroxide or triethylamine solution, a palladium catalyst (2-20 mole %) such as tetrakis(triphenylphosphine)-palladium (0) or [1,1′bis(diphenylphosphino)-ferrocene]dichloro-palladium(II), in a suitable solvent such as aqueous ethanol or THF or DMF or ethylene glycol for at temperatures ranging from 25° C. to 125° C. for 2-18 hr yields compound III. Alternatively, coupling reaction can be carried out by a conventional aryl or heteroaromatic coupling partner utilizing Stille coupling. e.g. Stille et al., Angew. Chem. Int. Ed. Engl., 1986, 25, 508.

Typical conditions used to carry out the Stille reaction include the use of an organostannane as the coupling partner, palladium catalyst (2-20 mole %) such as tetrakis(triphenylphosphine)-palladium (0) or [1,1′bis(diphenylphosphino)-ferrocene]dichloro-palladium(II), a salt such as potassium fluoride or lithium chloride, in a suitable anhydrous solvent such as THF or DMF or ethylene glycol for at temperatures ranging from 25° C. to 125° C. for 2-18 hr yields compound III.

Compound IV: The compound III is then further alkylated at to −50° C. to room temperature with suitable base such as sodium hydride and variety of halides (e.g., R₁ Br, R₂ Br, R₃ Br or R₁₁, R₂₁, R₃₁, where R₁, R₂, R₃ are defined above) yields mono-, di- or tri substituted compounds IV. The control of alkylation can be controlled by selecting the appropriate equivalence of the halide used. Scheme 3 below provides an alternatively general synthesis steps, and the examples provide a detailed description of the schematic methods.

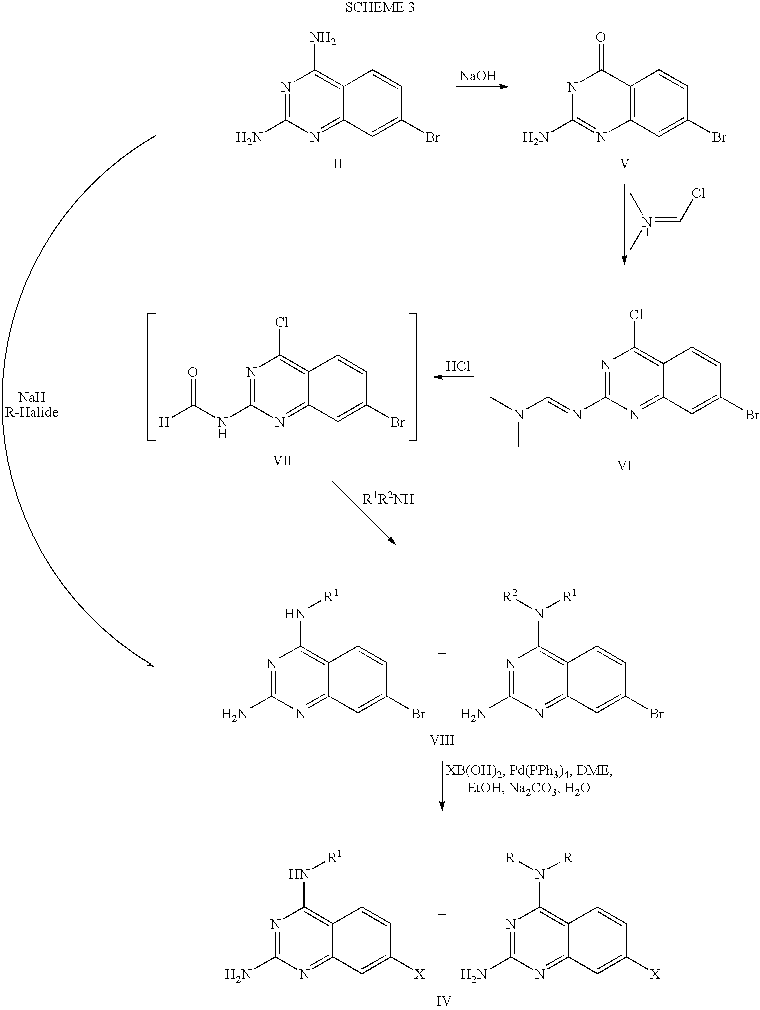

Compound V: A 5-L 3-neck Round bottom flask was charged with of 7-bromo-quinazoline-2,4-diamine II (292.9 g, 1225 mmol) followed by 2-methoxyethanol (600 mL) and a solution of sodium hydroxide (294 g, 7350 mmol) in deionized water (1.84 L). The mixture was then refluxed for 3 h, and a steady evolution of ammonia was noted. The yellow reaction solution was allowed to slowly cool to 65° C. overnight in order to avoid any accidental meltdowns. The resultant reaction was neutralized and acidified by drop wise addition of 12 N HCl (˜550 mL) with ice bath cooling to pH 5.5, and collected the white solid cake by filtration over a canvas filter pad. The solid cake was rinsed with ˜2 L of deionized water and sucked dry overnight at 25 torr at 60° C. with a slow nitrogen bleed to get 2-Amino-7-bromo-quinazolin-4-ol V (287.14 g, 96%) as a white powder. ¹H NMR (DMSO-d₆, 300 MHz) δ 11.16 (broad s, 1H), 7.74 (d, J=8.38 Hz, 1H), 7.73 (d, J=1.65 Hz, 1H), 7.18 (dd, J₁=8.38 Hz, J₂=1.65 Hz, 1H), 6.73 (broad s, 1H), 7.33 (d, J=1.95 Hz, 1H), 7.13 (dd, J₁=1.95 Hz, J₂=8.79 Hz, 1H), 6.18 (broad s, 2H).

Compound VI: Typical Vilsmeier chlorination conditions are carried out as described by Daluge et al, U.S. Pat. No. 5,917,042.

The Vilsmeier chlorination 2-Amino-quinazolin-4-ol may be carried out in an inert solvent such as toluene, chloroalkenes or chloroalkanes. The chlorination can be carried out at 0° C. to 100° C. Reaction time is typically 12-48 hours. of A 3-L three-neck Round bottom flask equipped was charged 2-Amino-7-bromo-quinazolin-4-ol V (33.60 g, 140 mmol), Chloroform (1.5 L) and Chloro methylenedimethyliminium chloride (58.51 g, 448 mmol, 3.2 equivalence), and warmed to reflux under N₂. The suspension became a yellow solution after ˜1 h and was refluxed while stirring for 16 hours with the evolved HCl gas being passed through an HCl scrubber. The resulting dark suspension was cooled to <5° C. and 800 mL of ice-water was added. Solid sodium carbonate monohydrate was added slowly until the pH reached ˜8. The layers were separated and the organic layer was washed with water (2×400 mL) followed brine (400 mL). The organic phase was dried over magnesium sulfate, filtered, and concentrated in vacuo to N′-(7-Bromo-4-chloro-quinazolin-2-yl)-N,N-dimethyl-formamidine VI as an orange solid (49.7 g). ¹H NMR (CDCl₃, 300 MHz) δ 8.82 (s, 1H), 7.89 (d, J=1.78 Hz, 1H), 7.94 (d, J=8.79 Hz, 1H), 7.48 (dd, J₁=8.79 Hz, J₂=1.78 Hz, 1H), 3.22 (s, 3H), 3.21 (s, 3H).

A solution of either 6N Hydrochloric acid or 1.5 M KH₂PO₄ that had been adjusted to pH 6 with 85% phosphoric acid is added to N′-(7-Bromo-4-chloro-quinazolin-2-yl)-N,N-dimethyl-formamidine VI, till the pH of the solution was ˜3, and the resultant reaction mixture is stirred for 24 h to 48 h at room temperature till hydrolysis of N′-(7-Bromo-4-chloro-quinazolin-2-yl)-N,N-dimethyl-formamidine VI to the N-(7-Bromo-4-chloro-quinazolin-2-yl)-formamide VII.

These mono- or di-substituted the 7-bromo-N4-alkyl-quinazoline-1,3-diamines VIII can be synthesized directly from the intermediate N-(7-Bromo-4-chloro-quinazolin-2-yl)-formamide VII by refluxing with an excess of amines (e.g., R₁R₂NH where R₁, R₂ are defined above) in the refluxing solvent such as ethanol, isopropanol, n-propanol, n-butanol and with similar reaction as described by Daluge et al, U.S. Pat. No. 5,917,042.

Compound IV: The coupling reaction can be carried out by a conventional aryl coupling method, e.g., Suzuki coupling method: (a) Suzuki et al., Synth. Commun. 1981, 11, 513, (b) Suzuki, Pure and Appl. Chem. 1985, 57, 1749-1758, (c) Suzuki et al., Chem. Rev. 1995, 95, 2457-2483, (d) Shieh et al., J. Org. Chem. 1992, 57, 379-381, (e) Martin et al., Acta Chemica Scandinavica. 1993, 47, 513.

Typical conditions used to carry out the Suzuki coupling of 7-bromo-N4-alkyl-quinazoline-1,3-diamine VIII include the use of either commercially appropriate aryl or heteroaromatic boronic acid or esters (e.g., where Ar is defined as aryl) as coupling partner, in aqueous base such as sodium bicarbonate or potassium carbonate or barium hydroxide or triethylamine solution, a palladium catalyst (2-20 mole %) such as tetrakis(triphenylphosphine)-palladium (0) or [1,1′-bis(diphenylphosphino)-ferrocene]dichloro-palladium(II), in a suitable solvent such as aqueous ethanol or THF or DMF or ethyleneglycol for at temperatures ranging from 25° C. to 125° C. for 2-18 hr yields compound the 7-aryl substituted N4-alkyl-quinazoline-1,3-diamine IV.

Alternatively, coupling reaction can be carried out by a conventional aryl or heteroaromatic coupling partner utilizing Stille coupling. e.g., Stille et al., Angew. Chem. Int. Ed. Engl., 1986, 25, 508.

Typical conditions used to carry out the Stille reaction include the use of an organostannane as the coupling partner, palladium catalyst (2-20 mole %) such as tetrakis(triphenylphosphine)-palladium (0) or [1,1′bis(diphenylphosphino)-ferrocene]dichloro-palladium(II), a salt such as potassium fluoride or lithium chloride, in a suitable anhydrous solvent such as THF or DMF or ethylene glycol for at temperatures ranging from 25° C. to 125° C. for 2-18 hr yields 6-aryl substituted 7-aryl substituted N4-alkyl-quinazoline-1,3-diamine IV.

Scheme 4 below provides one of the ways to synthesis appropriate substituted bromo aryl X that would be utilized as a coupling partner for Suzuki, Stille or other transition metal catalyzed coupling as described in scheme 7.

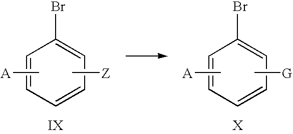

The appropriate substituted bromo aryl X Where A is hydrogen, halogen or mono or di or tri or tetra substituted alkyl, alkoxy, and where G is alkoxy, cyclic or acyclic amines can be made from commercially available bromo benzenes IX where A is hydrogen, halogen or mono or di or tri or tetra substituted alkyl, alkoxy, and where Z is mono or di or tri or tetra fluoro substituted compound via displacements of fluoride ion by an amine or alkoxy utilizing conventional fluoride displacement methods with a base such as potassium carbonate or cesium carbonates or sodium hydride in a suitable anhydrous solvent such as THF or DMF or DMSO or neat for at temperatures ranging from 25° C. to 125° C. for 2-18 hr. The fluoride displacement reactions are done according to the procedures reviewed by: (a) Vlasov, J. Fluorine Chem. 1993, 61, 193 (b) Clark, Chem. Rev. 1980, 80, 429 (c) Yakobson et al., Synthesis. 1983, 169 and the references cited in the mentioned reviews where IX possess multiple F, mono or di or tri or tetra substituted product can be obtained.

Scheme 5 below provides one of the ways to synthesis appropriate 2,6-hetero difunctional halo aryl XII that would be utilized as a coupling partner for Suzuki, Stille or other transition metal catalyzed coupling as described in scheme 7.

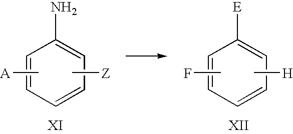

The appropriate substituted 2,6-hetero difunctional halo aryl XII where E is Bromo or Iodo, Where F is hydrogen, halogen or mono or di or tri or tetra substituted alkyl, alkoxy, thio alkoxy and where H is thio alkoxy, alkoxy, cyclic or acyclic amines can be made from commercially available anilines IX where A is hydrogen, halogen or mono or di or tri or tetra substituted alkyl, alkoxy, and where Z is mono or di or tri or tetra halogen substituted compound via diazotizations of anilines to respective halogens. The multi step synthesis of 2,6-hetero difunctional halo aryl XII was exactly carried out according to the procedures described by: Sienkowska M, Benin V, Kaszynki Tetrahedron 2000, 56, 1675 and the references cited in it.

Scheme 6 below provides one of the ways to synthesis appropriate 2,6 difunctional halo aryl XII that would be utilized as a coupling partner for Suzuki, Stille or other transition metal catalyzed coupling as described in Scheme 7

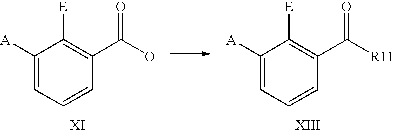

The appropriate substituted 2,6 difunctional halo aryl XIII where E is Bromo or Iodo, where A is hydrogen, halogen, lower alkyl, lower trifluoro alkyl, lower alkoxy, thio alkoxy, cyano, nitro, can be synthesised according from commercially available 2,6 difunctional benzoic acids XI according to literature procedures stated below:

Standard literature procedures for esterifications of carboxylic acids: see Smith, M.; March, J.; Advanced Organic Chemistry, Wiley-Interscience: NY, 2001, pp. 484-491 and the references cited in it.

Standard literature procedures for aminations of carboxylic acids: see Smith, M.; March, J.; Advanced Organic Chemistry, Wiley-Interscience: NY, 2001, pp. 508-515 and the references cited in it.

Standard literature procedures for aldehydes from carboxylic acids: see Smith, M.; March, J.; Advanced Organic Chemistry, Wiley-Interscience: NY, 2001, pp. 553-554 and the references cited in it.

Standard literature procedures for ketones: see Smith, M.; March, J.; Advanced Organic Chemistry, Wiley-Interscience: NY, 2001, pp. 1678-1680 and the references cited in it.

Non-commercially available 2,6 difunctional benzoic acids XI are prepared according to literature procedures as described by: (a) Huszthy, P; Kontos, Z; Vermes, B; Pinter, A. Tetrahedron (2001), 57, 4967-4975. (b) Denny, W. A; Atwell, G. J; Rewcastle, G. W; Baguley, B. C. J. Med. Chem. 1987, 30, 658-63. (C) Rewcastle, G. W; Denny, W. A. Synthesis 1985, 2, 217-30. (d) Atwell, G. J; Rewcastle, G. W; Baguley, B. C; Denny, W. A. J. Med. Chem. 1990, 33, 1375-9. (e) Mongin, F; Desponds, O; Schlosser, M. Tetrahedron Lett. 1996, 37, 2767-70.

Scheme 7 below provides another alternative enablement to make N4-alkyl-quinazoline-1,3-diamine IV from bromo aryl X or 2,6-hetero difunctional halo aryl XII or 2,6 difunctional halo aryl XIII via Suzuki or Stille coupling

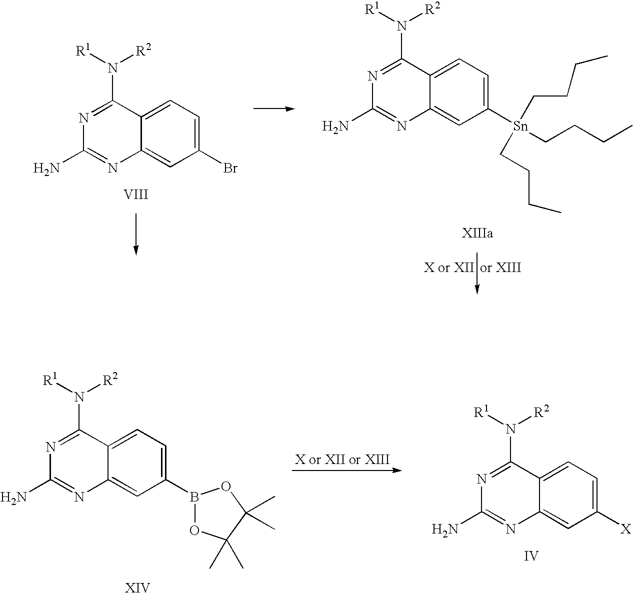

The organostannane XIIIa can be prepared by reacting mono- or di-substituted 7-bromo-N4-alkyl-quinazoline-1,3-diamines 7-bromo-N4-alkyl-quinazoline-1,3-diamine VIII to its coupling partner Bis (tributyl tin), palladium catalyst (2-20 mole %) such as tetrakis(triphenylphosphine)-palladium (0), a salt such as potassium fluoride or lithium chloride, in a suitable anhydrous solvent such as THF or DMF or ethylene glycol for at temperatures ranging from 25° C. to 125° C. for 2-18 hr.

The appropriate commercially available substituted bromo aryl X, XII, XIII (or prepared according to procedures described on (Scheme 4-6) can then be coupled to organo-stannane XIIIa via coupling reaction can be carried out by a conventional aryl or heteroaromatic coupling partner utilizing Stille coupling. e.g., Stille et al., Angew. Chem. Int. Ed. Engl., 1986, 25, 508.

Typical conditions used to carry out the Stille reaction include the use of an organostannane as the coupling partner, palladium catalyst (2-20 mole %) such as tetrakis(triphenylphosphine)-palladium (0) or [1,1′bis(diphenylphosphino)-ferrocene]dichloro-palladium(II), a salt such as potassium fluoride or lithium chloride, in a suitable anhydrous solvent such as THF or DMF or ethylene glycol for at temperatures ranging from 25° C. to 125° C. for 2-18 hr yields 6-aryl substituted 7-aryl substituted N4-alkyl-quinazoline-1,3-diamine IV.

Alternatively, the aryl[1,3,2]dioxaborolan XIV can be prepared by reacting mono- or di-substituted 7-bromo-N4-alkyl-quinazoline-1,3-diamine VIII to its coupling partner bis(pinacollato) diboran, palladium catalyst (2-20 mole %) such as tetrakis(triphenylphosphine)-palladium (0), a salt such as potassium acetate, in a suitable anhydrous solvent such as THF or DMF or ethylene glycol or DMSO for at temperatures ranging from 25° C. to 125° C. for 2-18 hr. e.g. (a) Suzuki et al., Synth. Commun. 1981, 11, 513, (b) Suzuki, Pure and Appl. Chem. 1985, 57, 1749-1758, (c) Suzuki et al., Chem. Rev. 1995, 95, 2457-2483, (d) Shieh et al., J. Org. Chem. 1992, 57, 379-381, (e) Martin et al., Acta Chemica Scandinavica. 1993, 47, 513.

The appropriate commercially available substituted bromo aryl X, XII, XIII (or prepared according to procedures described on (Scheme 4-6) can then be coupled to aryl[1,3,2]dioxaborolan XIV via coupling reaction utilizing e.g., Suzuki coupling method: (a) Suzuki et al., Synth. Commun. 1981, 11, 513, (b) Suzuki, Pure and Appl. Chem. 1985, 57, 1749-1758, (c) Suzuki et al., Chem. Rev. 1995, 95, 2457-2483, (d) Shieh et al., J. Org. Chem. 1992, 57, 379-381, (e) Martin et al., Acta Chemica Scandinavica. 1993, 47, 513.

In the examples which follow, the final compositions were purified either as free base or, utilizing reverse phase HPLC with TFA gradient, were prepared as the trifluoroacetic acid salt:

### EXAMPLES

### Example 1

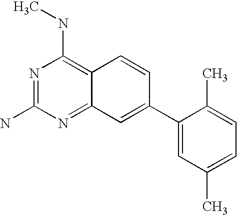

Prepared as described in Scheme 2, a solution of 7-bromo-quinazoline-2,4-diamine II (0.69 g, 2.886 mmol) in ethanol (25 mL) and ethylene glycol dimethyl ether (25 mL) was mixed with the tetrakis (triphenylphosphine) palladium(0) (0.71 g, 0.614 mmol), aq. saturated sodium carbonate solution (6.0 mL) and 2,5-dimethylphenyl boronic acid (0.86 g, 5.73 mmol) at room temperature under nitrogen. The resultant reaction mixture was heated at 85° C. for 1½ hours. The reaction was then cooled, diluted with water and extracted (3×100 mL) with 95:5:0.5 methylene chloride:methanol:aqueous ammonium hydroxide. The resultant combined organic layers were dried over anhydrous sodium sulfate, filtered and concentrated. Flash column chromatography on silica gel packed in 95:5:0.5 methylene chloride:methanol:aqueous. ammonium hydroxide. 7-(2,5-dimethyl-phenyl)-quinazoline-2,4-diamine (614.2 mg, 80.5%) was obtained as a light brown solid. ¹H NMR (DMSO-d₆, 400 MHz) δ 7.99 (d, J=8.79 Hz, 1H), 7.30 (broad s, 2H), 7.18 (d, J=7.81 Hz, 1H), 7.09 (d, J=7.82, 1H), 7.06 (broad s, 2H), 6.95 (dd, J₁=1.95, J₂=8.79, 1H), 6.02 (broad s, 2H), 2.30 (s, 3H), 2.19 (s, 3H).

A solution of 7-(2,5-dimethyl-phenyl)-quinazoline-2,4-diamine (510 mg, 1.929 mmol) and iodomethane (0.11 ml, 1.767 mmol) in anhydrous N,N-dimethylformamide (15 mL) at −5° C. was stirred under nitrogen and treated with 60% sodium hydride in oil dispersion (350 mg, 8.75 mmol) resulting a mild gas evolution. The resulting mixture was poured into water and extracted with a 9/1 methylene chloride/methanol solution. Flash chromatography (Merck Silica gel 60, 230-400 mesh, 9:1 methylene chloride/methanol) afforded 7-(2,5-dimethyl-phenyl)-N4-methyl-quinazoline-2,4-diamine (61.1 mg, 11.4%) as a pale yellow solid. ¹H NMR (DMSO-d₆, 400 MHz) δ 7.94 (d, J=8.79 Hz, 1H), 7.91 (broad q, J=3.91 Hz, 0.1H), 7.18 (d, J=7.81 Hz, 1H), 7.08 (m, 3H), 6.97 (dd, J₁=1.95 Hz, J₂=7.82 Hz 1H), 6.10 (broad s, 2H), 2.96 (d, J=3.91 Hz, 3H), 2.30 (s, 3H), 2.19 (s, 3H).

In an analogous manner, the compounds of Examples 2-29 were obtained as follows:

### Example 2

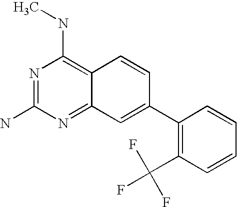

From 7-(2-trifluoromethyl-phenyl)-quinazoline-2,4-diamine there was produced N4-Methyl-7-(2-trifluoromethyl-phenyl)-quinazoline-2,4-diamine as an off-white solid; EI-HRMS m/e calcd for C₁₆H₁₃F₃N₄ (M⁺) 318.1092, found 318.1078.

### Example 3

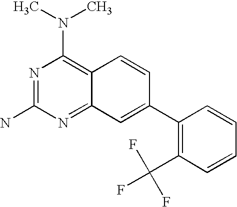

From 7-(2-trifluoromethyl-phenyl)-quinazoline-2,4-diamine there was produced N4,N4-Dimethyl-7-(2-trifluoromethyl-phenyl)-quinazoline-2,4-diamine trifluoroacetic acid salt as a white solid; EI-HRMS m/e calcd for C₁₇H₁₅F₃N₄ (M⁺) 332.1249, found 332.1240.

### Example 4

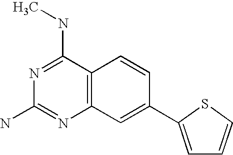

From 7-thiophen-2-yl-quinazoline-2,4-diamine there was produced N4-Methyl-7-thiophen-2-yl-quinazoline-2,4-diamine trifluoroacetic acid salt as a white solid; EI-HRMS m/e calcd for C₁₃H₁₂N₄S (M⁺) 256.0782, found 256.0790.

### Example 5

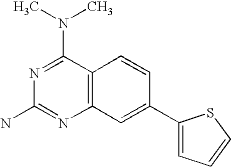

From 7-thiophen-2-yl-quinazoline-2,4-diamine there was produced N4,N4-Dimethyl-7-thiophen-2-yl-quinazoline-2,4-diamine trifluoroacetic acid salt as a white solid; EI-HRMS m/e calcd for C₁₄H₁₄N₄S (M⁺) 270.0939, found 270.0937.

### Example 6

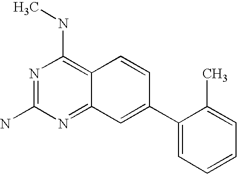

Prepared from 7-o-tolyl-quinazoline-2,4-diamine, there was produced N4-Methyl-7-o-tolyl-quinazoline-2,4-diamine as a white solid; EI-HRMS m/e calcd for C₁₆H₁₆N₄ (M⁺) 264.1375, found 264.1374.

Alternatively, in accordance with Scheme 3 a solution of 7-bromo-quinazoline-2,4-diamine II (4.02 g, 16.81

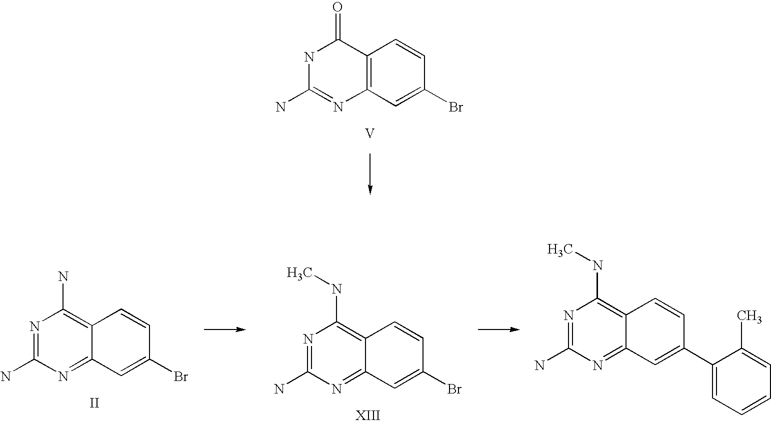

mmol) and iodomethane (0.95 mL, 15.26 mmol) in anhydrous N,N-dimethylformamide (90 mL) at −5° C. was stirred under nitrogen and treated with 60% sodium hydride in oil dispersion (3.1 g, 77.50 mmol) resulting a mild gas evolution. The resulting mixture was poured into water and extracted with a 9/1 methylene chloride/methanol solution. Flash chromatography (Merck Silica gel 60, 230-400 mesh, 9:1 methylene chloride/methanol) afforded 7-bromo-N4-methyl-quinazoline-2,4-diamine XIII (1.43 g, 33.6%) as an off-white solid. ¹H NMR (DMSO-d₆, 400 MHz) δ 7.99 (broad q, J=3.91 Hz, 1H), 7.84 (d, J=8.79 Hz, 1H), 7.33 (d, J=1.96 Hz, 1H), 7.15 (dd, J₁=1.96 Hz, J₂=8.79 Hz, 1H), 6.30 (broad s, 2H), 2.92 (d, J=4.88 Hz, 3H).

Alternatively, 7-bromo-N4-methyl-quinazoline-2,4-diamine IX was prepared as described as described in Scheme 3.

A 2-L 3-neck RBF equipped as above was charged with 2-Amino-7-bromo-quinazolin-4-ol V (72 g, 300 mmol), chloromethylene-dimethyliminium chloride (117.6 g, 900 mmol) and, dry CHCl₃ (900 mL), and refluxed for 25 h. The dark orange mixture was concentrated in vacuo to dryness, then added 1.5 M KH₂PO₄ (600 mL) that had been adjusted to pH 6 with 85% phosphoric acid. After addition, the pH of the solution was ˜3, and it was stirred for 2 h at RT. TLC analysis of the mixture still showed considerable intermediate, so the mixture was warmed to 60° C. for 2 h, and then allowed to stir at room temperature overnight. The mixture was brought to pH 6 with a saturated aqueous NaHCO₃ solution and the solids were collected by filtration, and then rinsed with water. The damp solid was combined with THF (400 mL) and 40% methylamine (200 mL) in water, and stirred at Room temperature for 1 h. Initially, the solids mostly went into solution, then the reaction rapidly thickened. The resultant reaction was warmed the mixture to 60° C. for 2 h. The reaction was then concentrated in vacuo to a dry slurry, and collected the solids by filtration. The solids were boiled with 600 mL of acetone/methanol mixture and the solids were collected by filtration. The filtrate was concentrated in vacuo to dryness. The residue (˜70 g) was dissolved in THF and added 87 g of Silica Gel 60 (230-400 mesh), then concentrated in vacuo to dryness. The residue was slurried with CH₂Cl₂ and applied to the top of a column of 517 g of Silica Gel 60 (230-400 mesh) packed in CH₂Cl₂. The column was eluted with methylene chloride followed by 10%, 20%, 25%, 40% methylene chloride in THF containing 1% NEt₃ affording 7-bromo-N4-methyl-quinazoline-2,4-diamine XIII (35.44 g, 46.7%) as an white solid. ¹H NMR (DMSO-d₆, 400 MHz) δ 7.99 (broad q, J=3.91 Hz, 1H), 7.84 (d, J=8.79 Hz, 1H), 7.33 (d, J=1.96 Hz, 1H), 7.15 (dd, J₁=1.96 Hz, J₂=8.79 Hz, 1H), 6.30 (broad s, 2H), 2.92 (d, J=4.88 Hz, 3H).

A mixture of 7-bromo-N4-methyl-quinazoline-2,4-diamine XIII (4.35 g, 17.19 mmol), ethanol (80 mL), ethylene glycol dimethyl ether (80 mL), tetrakis(triphenylphoshpine)palladium(0) (4.37 g, 3.782 mmol), aq. sat. sodium carbonate solution (30 mL) and 2-tolylboronic acid (4.09 g, 30.08 mmol) was heated at reflux for 2 hours. The resultant reaction mixture was then cooled, diluted with water, extracted 90:10:1 methylene chloride:methanol:ammonium hydroxide (3×200 mL). The combined Organic layers were dried over anhydrous sodium sulfate, filtered, added silica gel (5 gm), and concentrated. Flash column chromatography on silica gel packed with 95:5:0.5 methylene chloride:methanol:ammonium hydroxide and eluted with same to yield N4-methyl-7-o-tolyl-quinazoline-2,4-diamine (3.54 g, 77.9%) as an off white foam. ¹H NMR (DMSO-d₆, 400 MHz) δ 7.95 (d, J=8.79 Hz, 1H), 7.93 (broad q, J=4.89, 1H), 7.27 (m, 4H), 7.08 (d, J=1.95 Hz, 1H), 6.99 (dd, J₁=1.95 Hz, J₂=7.82 Hz, 1H), 6.14 (broad s, 2H), 2.96 (d, J=4.88 Hz, 3H), 2.25 (s, 3H).

### Example 7

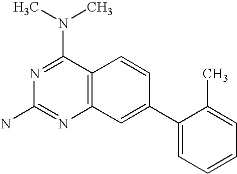

From 7-o-tolyl-quinazoline-2,4-diamine there was produced N4,N4-Dimethyl-7-o-tolyl-quinazoline-2,4-diamine trifluoroacetic acid salt as a white solid; EI-HRMS m/e calcd for C₁₇H₁₈N₄ (M⁺) 278.1531, found 278.1531.

### Example 8

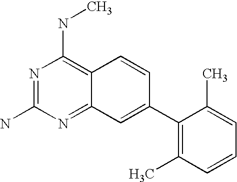

From 7-(2,6-Dimethyl-phenyl)-quinazoline-2,4-diamine there was produced 7-(2,6-Dimethyl-phenyl)-N4-methyl-quinazoline-2,4-diamine as an off-white solid; EI-HRMS m/e calcd for C₁₇H₁₈N₄ (M⁺) 278.1531, found 278.1528.

### Example 9

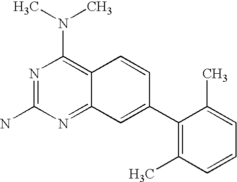

From 7-(2,6-Dimethyl-phenyl)-quinazoline-2,4-diamine there was produced 7-(2,6-Dimethyl-phenyl)-N4,N4-dimethyl-quinazoline-2,4-diamine as a white solid; EI-HRMS m/e calcd for C₁₈H₂₀N₄ (M⁺) 292.1688, found 292.1692.

### Example 10

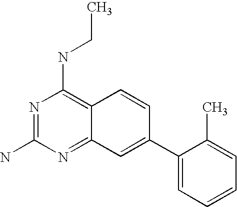

From 7-o-tolyl-quinazoline-2,4-diamine there was produced N4-Ethyl-7-o-tolyl-quinazoline-2,4-diamine trifluoroacetic acid salt as an off-white solid; EI-HRMS m/e calcd for C₁₇H₁₈N₄ (M⁺) 278.1531, found 278.1531.

### Example 11

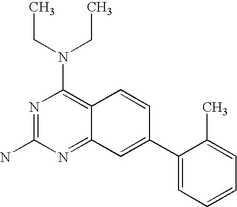

From 7-o-tolyl-quinazoline-2,4-diamine there was produced N4,N4-Diethyl-7-o-tolyl-quinazoline-2,4-diamine trifluoroacetic acid salt as an off-white solid; EI-HRMS m/e calcd for C₁₉H₂₂N₄ (M⁺) 306.1844, found 306.1838.

### Example 12

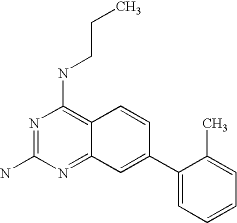

From 7-o-tolyl-quinazoline-2,4-diamine there was produced N4-Propyl-7-o-tolyl-quinazoline-2,4-diamine trifluoroacetic acid salt as an off-white solid; EI-HRMS m/e calcd for C₁₈H₂₀N₄ (M⁺) 292.1688, found 292.1681.

### Example 13

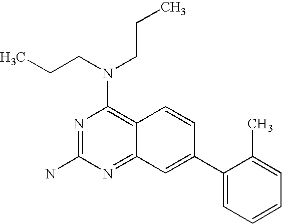

From 7-o-tolyl-quinazoline-2,4-diamine there was produced N4,N4-Dipropyl-7-o-tolyl-quinazoline-2,4-diamine trifluoroacetic acid salt as a tan solid; EI-HRMS m/e calcd for C₂₁H₂₆N₄ (M⁺) 334.2157, found 334.2150.

### Example 14

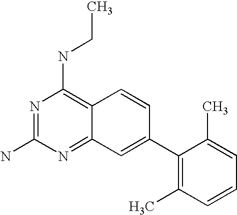

From 7-(2,6-Dimethyl-phenyl)-quinazoline-2,4-diamine there was produced 7-(2,6-Dimethyl-phenyl)-N4-ethyl-quinazoline-2,4-diamine trifluoroacetic acid salt as a white solid; EI-HRMS m/e calcd for C₁₈H₂₀N₄ (M⁺) 292.1688, found 292.1691.

### Example 15

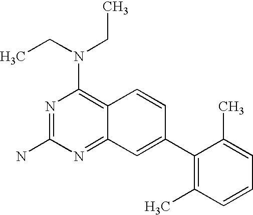

From 7-(2,6-Dimethyl-phenyl)-quinazoline-2,4-diamine there was produced 7-(2,6-Dimethyl-phenyl)-N4,N4-diethyl-quinazoline-2,4-diamine trifluoroacetic acid salt as a white solid; EI-HRMS m/e calcd for C₂₀H₂₄N₄ (M⁺) 320.2001, found 320.2007.

### Example 16

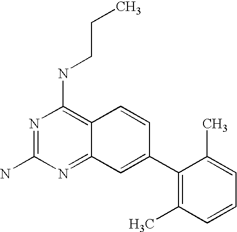

From 7-(2,6-Dimethyl-phenyl)-quinazoline-2,4-diamine there was produced 7-(2,6-Dimethyl-phenyl)-N4-propyl-quinazoline-2,4-diamine trifluoroacetic acid salt as a white solid; EI-HRMS m/e calcd for C₁₉H₂₂N₄ (M⁺) 306.1844, found 306.1838.

### Example 17

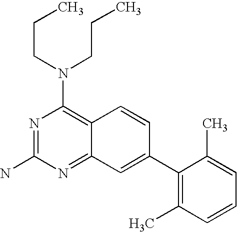

From 7-(2,6-Dimethyl-phenyl)-quinazoline-2,4-diamine there was produced 7-(2,6-Dimethyl-phenyl)-N4,N4-dipropyl-quinazoline-2,4-diamine trifluoroacetic acid salt as a white solid; EI-HRMS m/e calcd for C₂₂H₂₈N₄ (M⁺) 348.2314, found 348.2317.

### Example 18

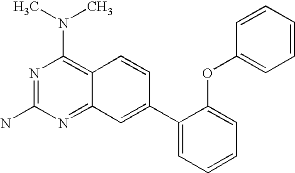

From 7-(2-phenoxy-phenyl)-quinazoline-2,4-diamine there was produced N4,N4-Dimethyl-7-(2-phenoxy-phenyl)-quinazoline-2,4-diamine as an off-white solid; EI-HRMS m/e calcd for C₂₂H₂₀N₄O (M⁺) 356.1639, found 356.1637.

### Example 19

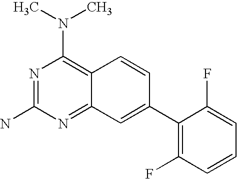

From 7-(2,6-Difluoro-phenyl)-quinazoline-2,4-diamine there was produced 7-(2,6-Difluoro-phenyl)-N4,N4-dimethyl-quinazoline-2,4-diamine as a light yellow solid; EI-HRMS m/e calcd for C₁₆H₁₄F₂N₄ (M⁺) 300.1186, found 300.1185.

### Example 20

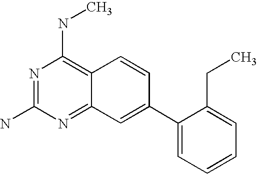

From 7-(2-Ethyl-phenyl)-quinazoline-2,4-diamine there was produced 7-(2-Ethyl-phenyl)-N4-methyl-quinazoline-2,4-diamine as an off-white solid; EI-HRMS m/e calcd for C₁₇H₁₈N₄ (M⁺) 278.1531, found 278.1530.

### Example 21

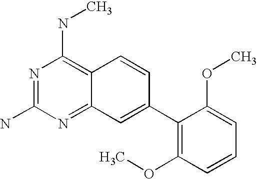

From 7-(2,6-Dimethoxy-phenyl)-quinazoline-2,4-diamine there was produced 7-(2,6-Dimethoxy-phenyl)-N4-methyl-quinazoline-2,4-diamine as a tan solid; EI-HRMS m/e calcd for C₁₇H₁₈N₄O₂ (M⁺) 310.1430, found 310.1429.

### Example 22

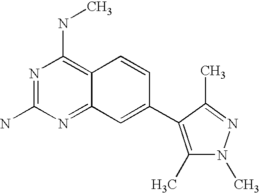

Prepared as described in Scheme 6, by utilizing Stille coupling, from N4-methyl-7-tributylstannanyl-quinazoline-2,4-diamine and 4-Iodo-1,3,5-trimethyl-1H-pyrazole: N4-Methyl-7-(1,3,5-trimethyl-1H-pyrazol-4-yl)-quinazoline-2,4-diamine as an off-white solid; LRMS for C₁₅H₁₈N₆ (M+H)⁺ at m/z=283.

### Example 23

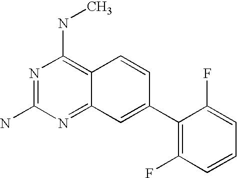

From 7-(2,6-Difluoro-phenyl)-quinazoline-2,4-diamine there was produced 7-(2,6-Difluoro-phenyl)-N4-methyl-quinazoline-2,4-diamine as a pale yellow solid; EI-HRMS m/e calcd for C₁₅H₁₂F₂N₄(M⁺)286.1030, found 286.1034.

### Example 24

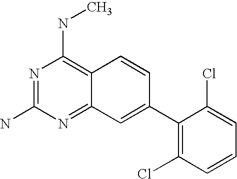

From 7-(2,6-Dichloro-phenyl)-quinazoline-2,4-diamine there was produced 7-(2,6-Dichloro-phenyl)-N4-methyl-quinazoline-2,4-diamine as a pale yellow solid; EI-HRMS m/e calcd for C₁₅H₁₂Cl₂N₄ (M⁺) 318.0439, found 318.0436.

### Example 25

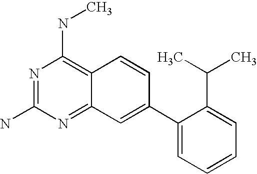

From 7-(2-Isopropyl-phenyl)-quinazoline-2,4-diamine there was produced 7-(2-Isopropyl-phenyl)-N4-methyl-quinazoline-2,4-diamine as an off-white solid; EI-HRMS m/e calcd for C₁₈H₂₀N₄ (M⁺) 292.1688, found 292.1689.

### Example 26

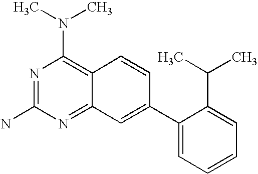

From 7-(2-Isopropyl-phenyl)-quinazoline-2,4-diamine there was produced 7-(2-Isopropyl-phenyl)-N4,N4-dimethyl-quinazoline-2,4-diamine as an off-white solid; EI-HRMS m/e calcd for C₁₉H₂₂N₄ (M⁺) 306.1844, found 306.1845.

### Example 27

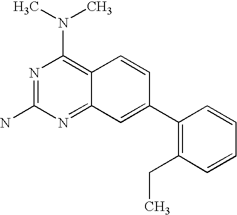

From 7-(2-Ethyl-phenyl)-quinazoline-2,4-diamine there was produced 7-(2-Ethyl-phenyl)-N4,N4-dimethyl-quinazoline-2,4-diamine as a light yellow solid; EI-HRMS m/e calcd for C₁₈H₂₀N₄ (M⁺) 292.1688, found 292.1680.

### Example 28

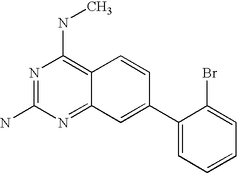

From 7-bromo-N4-methyl-quinazoline-2,4-diamine and 2-Bromo-phenyl boronic acid: 7-(2-bromo-phenyl)-N4-methyl-quinazoline-2,4-diamine as an off-white solid; EI-LRMS for C₁₅H₁₃BrN₄ (M+H)⁺ at m/z=330.

### Example 29

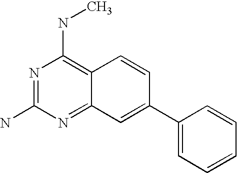

From 7-bromo-N4-methyl-quinazoline-2,4-diamine and 2-phenyl boronic acid there was produced: N4-Methyl-7-phenyl-quinazoline-2,4-diamine trifluoroacetic acid salt as a white solid; EI-HRMS m/e calcd for C₁₅H₁₄N₄ (M⁺) 250.1218, found 250.1215.

### Example 30

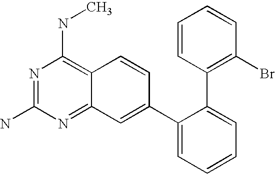

From 7-bromo-N4-methyl-quinazoline-2,4-diamine and 2-Bromo-phenyl boronic acid: 7-(2′-Bromo-biphenyl-2-yl)-4-methyl-quinazoline-2,4-diamine as an off-white solid; EI-LRMS for C₂₁H₁₇BrN₄ (M+H)⁺ at m/z=406.

### Example 31

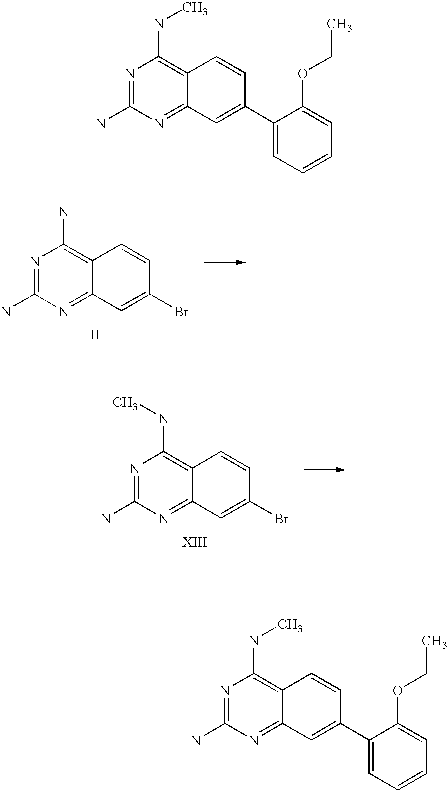

A 2-L 3-neck round bottom flask equipped as above was charged with 2-Amino-7-bromo-quinazolin-4-ol V (72 g, 300 mmol), prepared as described in scheme 3, chloromethylene-dimethyliminium chloride (117.6 g, 900 mmol) and, dry CHCl₃ (900 mL), and refluxed for 25 h. The dark orange mixture was concentrated in vacuo to dryness, then added 1.5 M KH₂PO₄ (600 mL) that had been adjusted to pH 6 with 85% phosphoric acid. After addition, the pH of the solution was ˜3, and it was stirred for 2 h at RT. TLC analysis of the mixture still showed considerable intermediate, so the mixture was warmed to 60° C. for 2 h, and then allowed to stir at room temperature overnight. The mixture was brought to pH 6 with a saturated aqueous NaHCO₃ solution and the solids were collected by filtration, and then rinsed with water. The damp solid was combined with THF (400 mL) and 40% methylamine (200 mL) in water, and stirred at Room temperature for 1 h. Initially, the solids mostly went into solution, and then the reaction rapidly thickened. The resultant reaction was warmed the mixture to 60° C. for 2 h. The reaction was then concentrated in vacuo to a dry slurry, and collected the solids by filtration. The solids were boiled with 600 mL of acetone/methanol mixture and the solids were collected by filtration. The filtrate was concentrated in vacuo to dryness. The residue (˜70 g) was dissolved in THF and added 87 g of Silica Gel 60 (230-400 mesh), then concentrated in vacuo to dryness. The residue was slurried with CH₂Cl₂ and applied to the top of a column of 517 g of Silica Gel 60 (230-400 mesh) packed in CH₂Cl₂. The column was eluted with methylene chloride followed by 10%, 20%, 25%, 40% methylene chloride in THF containing 1% triethylamine affording 7-bromo-N4-methyl-quinazoline-2,4-diamine XIII (35.44 g, 46.7%) as an white solid. ¹H NMR (DMSO-d₆, 400 MHz) δ 7.99 (broad q, J=3.91 Hz, 1H), 7.84 (d, J=8.79 Hz, 1H), 7.33 (d, J=1.96 Hz, 1H), 7.15 (dd, J₁=1.96 Hz, J₂=8.79 Hz, 1H), 6.30 (broad s, 2H), 2.92 (d, J=4.88 Hz, 3H).

A mixture of 7-bromo-N4-methyl-quinazoline-2,4-diamine XIII (0.300 g, 1.186 mmol), ethanol (20 mL), ethylene glycol dimethyl ether (20 mL), tetrakis(triphenylphoshpine) palladium(0) (0.293 g, 0.254 mmol), aq. sat. sodium carbonate solution (3 mL) and 2-Ethoxy-phenyl boronic acid (0.3937 g, 2.372 mmol) was heated at reflux for 2 hours. The resultant reaction mixture was then cooled, diluted with water, extracted 90:10:1 methylene chloride:methanol:ammonium hydroxide (3×200 mL). The combined Organic layers were dried over anhydrous sodium sulfate, filtered, added silica gel (5 gm), and concentrated. Flash column chromatography on silica gel packed with 95:5:0.5 methylene chloride:methanol:ammonium hydroxide and eluted with same to yield N4-methyl-7-o-tolyl-quinazoline-2,4-diamine (0.100 g, 28.7%) as an off white foam. EI-HRMS m/e calcd for C₁₇H₁₈N₄O (M⁺) 294.1481, found 294.1483. In an analogous manner as described in Scheme 3 and example 31, the following examples were obtained:

### Example 32

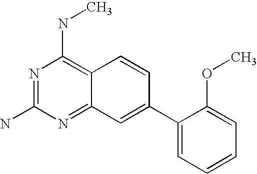

From 7-bromo-N4-methyl-quinazoline-2,4-diamine and 2-Methoxy-phenyl boronic acid there was produced 7-(2-Methoxy-phenyl)-N4-methyl-quinazoline-2,4-diamine as an off-white solid; EI-HRMS m/e calcd for C₁₆H₁₆N₄O (M⁺) 280.1324, found 280.1329.

### Example 33

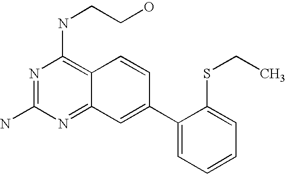

From 2-[2-Amino-7-bromo-quinazolin-4-ylamino]-ethanol and 2-ethylsulfanyl-phenyl boronic acid: 2-[2-Amino-7-(2-ethylsulfanyl-phenyl)-quinazolin-4-ylamino]-ethanol as an off-white solid; EI-HRMS m/e calcd for C₁₈H₂₀N₄OS (M⁺) 340.1358, found 340.1355.

### Example 34

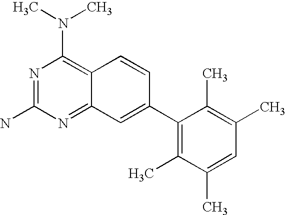

From 7-bromo-N4,N4-dimethyl-quinazoline-2,4-diamine and 2,3,5,6-tetramethyl-phenyl boronic acid there was produced N4,N4-Dimethyl-7-(2,3,5,6-tetramethyl-phenyl)-quinazoline-2,4-diamine as an off-white solid; EI-HRMS m/e calcd for C₂₀H₂₄N₄ (M⁺) 320.2001, found 320.1996.

### Example 35

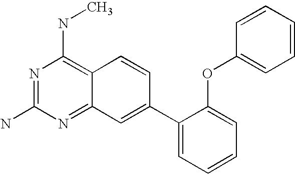

From 7-bromo-N4-methyl-quinazoline-2,4-diamine and 2-phenoxy-phenyl boronic acid there was produced N4-Methyl-7-(2-phenoxy-phenyl)-quinazoline-2,4-diamine as an off-white solid; EI-HRMS m/e calcd for C₂₁H₁₈N₄O (M⁺) 342.1481, found 342.1476.

### Example 36

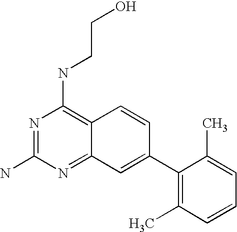

From 2-[2-Amino-7-bromo-quinazolin-4-ylamino]-ethanol and 2,6-dimethyl-phenyl boronic acid: there was produced 2-[2-Amino-7-(2,6-dimethyl-phenyl)-quinazolin-4-ylamino]-ethanol as a tan solid; (ES)⁺-HRMS m/e calcd for C₁₈H₂₀N₄O (M+H)⁺ 309.1710, found 309.1714.

### Example 37

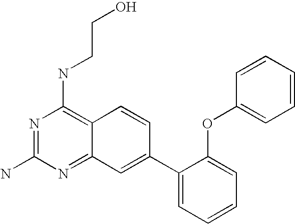

From 2-[2-Amino-7-bromo-quinazolin-4-ylamino]-ethanol and 2-phenoxy-phenyl boronic acid: there was produced 2-[2-Amino-7-(2-phenoxy-phenyl)-quinazolin-4-ylamino]-ethanol as an off-white solid; (ES)⁺-HRMS m/e calcd for C₂₂H₂₀N₄O₂ (M+H)⁺ 373.1659, found 373.1661.

### Example 38

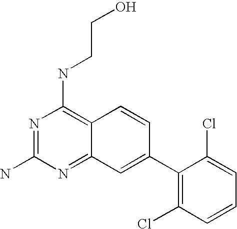

From 2-[2-Amino-7-bromo-quinazolin-4-ylamino]-ethanol and 2,6-dichloro-phenyl boronic acid: there was produced 2-[2-Amino-7-(2,6-dichloro-phenyl)-quinazolin-4-ylamino]-ethanol as a light grey solid; (ES)⁺-HRMS m/e calcd for C₁₆H₁₄Cl₂N₄O (M+H)⁺ 349.0618, found 349.0619.

### Example 39

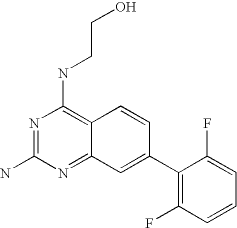

From 2-[2-Amino-7-bromo-quinazolin-4-ylamino]-ethanol and 2,6-difluoro-phenyl boronic acid: there was produced 2-[2-Amino-7-(2,6-difluoro-phenyl)-quinazolin-4-ylamino]-ethanol as an off white solid; EI-HRMS m/e calcd for C₁₆H₁₄F₂N₄O (M⁺) 316.1136, found 316.1121.

### Example 40

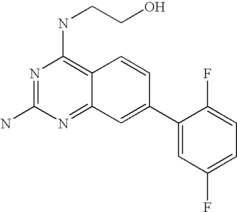

From 2-[2-Amino-7-bromo-quinazolin-4-ylamino]-ethanol and 2,5-difluoro-phenyl boronic acid: there was produced 2-[2-Amino-7-(2,5-difluoro-phenyl)-quinazolin-4-ylamino]-ethanol as an off-white solid; EI-HRMS m/e calcd for C₁₆H₁₄F₂N₄O (M⁺) 316.1136, found 316.1134.

### Example 41

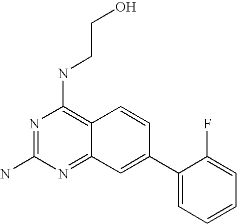

From 2-[2-Amino-7-bromo-quinazolin-4-ylamino]-ethanol and 2-fluoro-phenyl boronic acid: there was produced 2-[2-Amino-7-(2-fluoro-phenyl)-quinazolin-4-ylamino]-ethanol as an off-white solid; EI-HRMS m/e calcd for C₁₆H₁₅FN₄O (M⁺) 298.1229, found 298.1227.

### Example 42

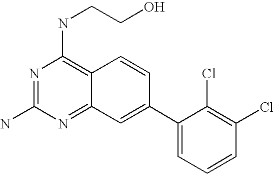

From 2-[2-Amino-7-bromo-quinazolin-4-ylamino]-ethanol and 2,3-dichloro-phenyl boronic acid: there was produced 2-[2-Amino-7-(2,3-dichloro-phenyl)-quinazolin-4-ylamino]-ethanol as an off-white solid; EI-HRMS m/e calcd for C₁₆H₁₄Cl₂N₄O (M⁺) 348.0545, found 348.0543.

### Example 43

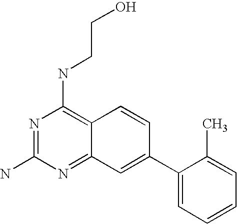

From 2-[2-Amino-7-bromo-quinazolin-4-ylamino]-ethanol and o-tolyl boronic acid: there was produced 2-(2-Amino-7-o-tolyl-quinazolin-4-ylamino)-ethanol as a light brown solid; EI-HRMS m/e calcd for C₁₇H₁₈N₄O (M⁺) 294.1481, found 294.1485.

### Example 44

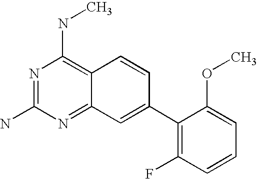

From N4-methyl-quinazoline-2,4-diamine and 2-fluoro-6-methoxyphenylboronic acid: 7-(2-Fluoro-6-methoxy-phenyl)-N4-methyl-quinazoline-2,4-diamine as a white powder; LRMS for C₁₆H₁₅FN₄O (M+H)⁺ at m/z=299.

### Example 45

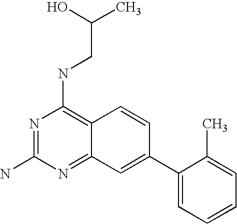

From 1-[2-Amino-7-bromo-quinazolin-4-ylamino]-propan-2-ol and o-tolyl boronic acid: there was produced 1-(2-Amino-7-o-tolyl-quinazolin-4-ylamino)-propan-2-ol as an off-white solid; (ES)⁺-HRMS m/e calcd for C₁₈H₂₀N₄O (M+H)⁺ 309.1710, found 309.1713.

### Example 46

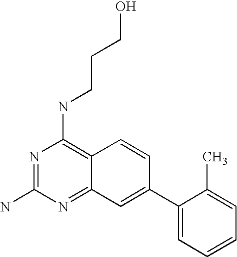

From 2-[2-Amino-7-bromo-quinazolin-4-ylamino]-propan-1-ol and o-tolyl boronic acid: 3-(2-Amino-7-o-tolyl-quinazolin-4-ylamino)-propan-1-ol as an orange solid; (ES)⁺-HRMS m/e calcd for C₁₈H₂₀N₄O (M+H)⁺ 309.1710, found 309.1712.

### Example 47

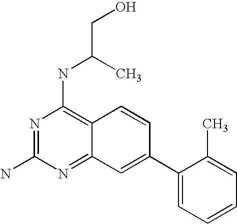

From 2-[2-Amino-7-bromo-quinazolin-4-ylamino]-propan-1-ol and o-tolyl boronic acid: there was produced 2-(2-Amino-7-o-tolyl-quinazolin-4-ylamino)-propan-1-ol as an off-white solid; (ES)⁺-HRMS m/e calcd for C₁₈H₂₀N₄O (M+H)⁺ 309.1710, found 309.1714.

### Example 48

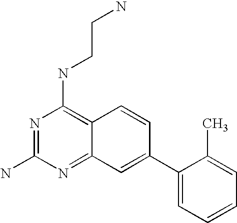

From N4-[2-Amino-ethyl]-7-bromo-quinazolin-2,4-diamine and o-tolyl boronic acid: there was produced N4-(2-Amino-ethyl)-7-o-tolyl-quinazoline-2,4-diamine as a white solid; (ES)⁺-HRMS m/e calcd for C₁₇H₁₉N₅ (M+H)⁺ 294.1713, found 294.1713.

### Example 49

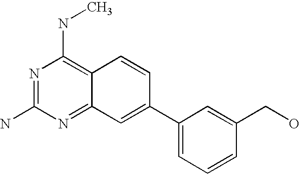

From 7-bromo-N4-methyl-quinazoline-2,4-diamine and 3-methyl alcohol-phenyl boronic acid: there was produced [3-(2-Amino-4-methylamino-quinazolin-7-yl)-phenyl]-methanol trifluoroacetic acid salt as a white solid; (ES)⁺-HRMS m/e calcd for C₁₆H₁₆N₄O (M+H)⁺ 281.1397, found 281.1401.

### Example 50

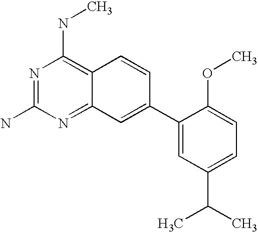

From 7-bromo-N4-methyl-quinazoline-2,4-diamine and 5-Isopropyl-2-methoxy-phenyl boronic acid: there was produced 7-(5-Isopropyl-2-methoxy-phenyl)-N4-methyl-quinazoline-2,4-diamine trifluoroacetic acid salt as a light brown solid; (ES)⁺-HRMS m/e calcd for C₁₉H₂₂N₄O (M+H)⁺ 323.1867, found 323.1870.

### Example 51

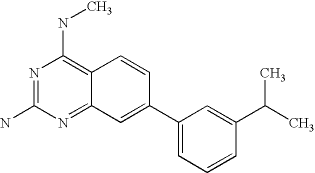

From 7-bromo-N4-methyl-quinazoline-2,4-diamine and 3-Isopropyl-phenyl boronic acid: there was produced 7-(3-Isopropyl-phenyl)-N4-methyl-quinazoline-2,4-diamine trifluoroacetic acid salt as a light grey solid; (ES)⁺-HRMS m/e calcd for C₁₈H₂₀N₄ (M+H)⁺ 293.1761, found 293.1765.

### Example 52

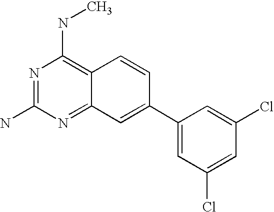

From 7-bromo-N4-methyl-quinazoline-2,4-diamine and 3,5-Dichloro-phenyl boronic acid: 7-(3,5-Dichloro-phenyl)-N4-methyl-quinazoline-2,4-diamine trifluoroacetic acid salt as a light grey solid; (ES)⁺-HRMS m/e calcd for C₁₅H₁₂Cl₂N₄ (M+H)⁺ 319.0512, found 319.0514.

### Example 53

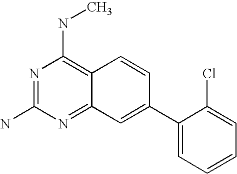

From 7-bromo-N4-methyl-quinazoline-2,4-diamine and 2-Chloro-phenyl boronic acid: there was produced 7-(2-Chloro-phenyl)-N4-methyl-quinazoline-2,4-diamine trifluoroacetic acid salt as a white solid; (ES)⁺-HRMS m/e calcd for C₁₅H₁₃ClN₄ (M+H)⁺ 285.0902, found 285.0906.

### Example 54

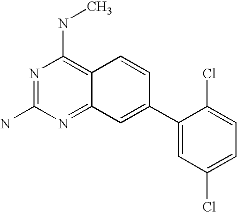

From 7-bromo-N4-methyl-quinazoline-2,4-diamine and 2,5-Dichloro-phenyl boronic acid: there was produced 7-(2,5-Dichloro-phenyl)-N4-methyl-quinazoline-2,4-diamine trifluoroacetic acid salt as a white solid; (ES)⁺-HRMS m/e calcd for C₁₅H₁₂Cl₂N₄ (M+H)⁺ 319.0512, found 319.0514.

### Example 55

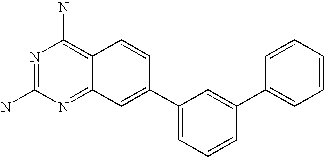

From 7-bromo-N4-methyl-quinazoline-2,4-diamine and 3-phenyl-phenyl boronic acid: there was produced 7-Biphenyl-3-yl-N4-methyl-quinazoline-2,4-diamine trifluoroacetic acid salt as a white solid; (ES)⁺-HRMS m/e calcd for C₂₁H₁₈N₄ (M+H)⁺ 327.1604, found 327.1607.

### Example 56

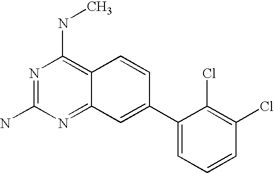

From 7-bromo-N4-methyl-quinazoline-2,4-diamine and 2,3-Dichloro-phenyl boronic acid: there was produced 7-(2,3-Dichloro-phenyl)-N4-methyl-quinazoline-2,4-diamine trifluoroacetic acid salt as a tan solid; (ES)⁺-HRMS m/e calcd for C₁₅H₁₂Cl₂N₄ (M+H)⁺ 319.0512, found 319.0514.

### Example 57

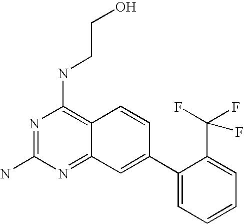

From 2-[2-Amino-7-bromo-quinazolin-4-ylamino]-ethanol and 2-trifluoromethyl-phenyl boronic acid: there was produced 2-[2-Amino-7-(2-trifluoromethyl-phenyl)-quinazolin-4-ylamino]-ethanol as an off-white solid; (ES)⁺-HRMS m/e calcd for C₁₇H₁₅F₃N₄O (M+H)⁺ 349.1271, found 349.1273.

### Example 58

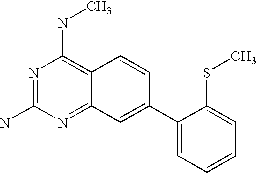

From 7-bromo-N4-methyl-quinazoline-2,4-diamine and 2-methylsulfanyl-phenyl boronic acid: there was produced N4-Methyl-7-(2-methylsulfanyl-phenyl)-quinazoline-2,4-diamine as a white solid; (ES)⁺-HRMS m/e calcd for C₁₆H₁₆N₄S (M+H)⁺ 297.1169, found 297.1168.

### Example 59

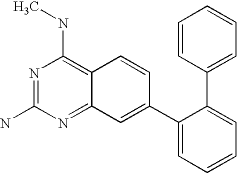

From 7-bromo-N4-methyl-quinazoline-2,4-diamine and 2-phenyl-phenyl boronic acid: there was produced 7-Biphenyl-2-yl-N4-methyl-quinazoline-2,4-diamine trifluoroacetic acid salt as an orange solid; (ES)⁺-HRMS m/e calcd for C₂₁H₁₈N₄ (M+H)⁺ 327.1604, found 327.1607.

### Example 60

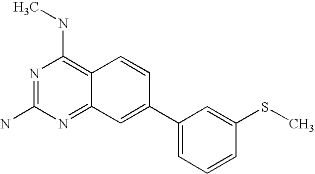

From 7-bromo-N4-methyl-quinazoline-2,4-diamine and 3-methylsulfanyl-phenyl boronic acid: there was produced N4-Methyl-7-(3-methylsulfanyl-phenyl)-quinazoline-2,4-diamine trifluoroacetic acid salt as an off-white solid; (ES)⁺-HRMS m/e calcd for C₁₆H₁₆N₄S (M+H)⁺ 297.1169, found 297.1171.

### Example 61

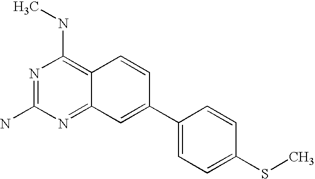

From 7-bromo-N4-methyl-quinazoline-2,4-diamine and 4-methylsulfanyl-phenyl boronic acid: there was produced N4-Methyl-7-(4-methylsulfanyl-phenyl)-quinazoline-2,4-diamine trifluoroacetic acid salt as a white solid; (ES)⁺-HRMS m/e calcd for C₁₆H₁₆N₄S (M+H)⁺ 297.1169, found 297.1173.

### Example 62

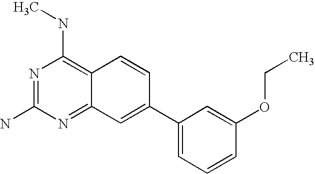

From 7-bromo-N4-methyl-quinazoline-2,4-diamine and 3-Ethoxy-phenyl boronic acid: there was produced 7-(3-Ethoxy-phenyl)-N4-methyl-quinazoline-2,4-diamine trifluoroacetic acid salt as a white solid; (ES)⁺-HRMS m/e calcd for C₁₇H₁₈N₄O (M+H)⁺ 295.1554, found 295.1557.

### Example 63

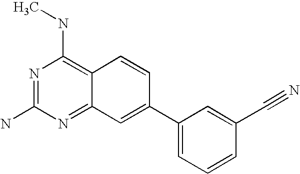

From 7-bromo-N4-methyl-quinazoline-2,4-diamine and 3-cyano-phenyl boronic acid: there was produced 3-(2-Amino-4-methylamino-quinazolin-7-yl)-benzonitrile trifluoroacetic acid salt as a white solid; (ES)⁺-HRMS m/e calcd for C₁₆H₁₃N₅ (M+H)⁺ 276.1244, found 276.1247.

### Example 64

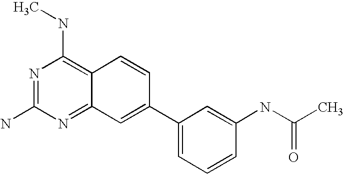

From 7-bromo-N4-methyl-quinazoline-2,4-diamine and 3-acetamide-phenyl boronic acid: there was produced N-[3-(2-Amino-4-methylamino-quinazolin-7-yl)-phenyl]-acetamide trifluoroacetic acid salt as an off-white solid; (ES)⁺-HRMS m/e calcd for C₁₇H₁₇N₅O (M+H)⁺ 308.1506, found 308.1507.

### Example 65

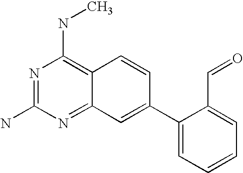

From 7-Bromo-N4-methyl-quinazoline-2,4-diamine) and 2-formylphenylboronic: there was produced 2-(2-amino-4-methylamino-quinazolin-7-yl)-benzaldehyde as a light yellow solid; LRMS for C₁₆H₁₄N₄O (M+H)⁺ at m/z=279.

### Example 66

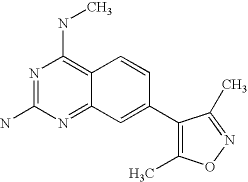

From 7-bromo-N4-methyl-quinazoline-2,4-diamine and 3,5-Dimethyl-isoxazol-4-yl boronic acid: there was produced 7-(3,5-Dimethyl-isoxazol-4-yl)-N4-methyl-quinazoline-2,4-diamine as an off-white solid; EI-HRMS m/e calcd for C₁₄H₁₅N₅O (M⁺) 269.1277, found 269.1269.

### Example 67

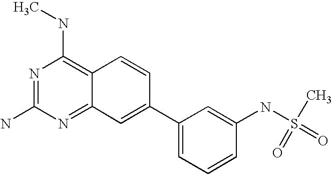

From 7-bromo-N4-methyl-quinazoline-2,4-diamine: there was produced N-[3-(2-Amino-4-methylamino-quinazolin-7-yl)-phenyl]-methanesulfonamide trifluoroacetic acid salt as a white powder; (ES)⁺-HRMS m/e calcd for C₁₆H₁₇N₅O₂S (M+H)⁺ 344.1174, found 344.1176.

### Example 68

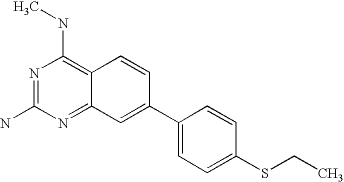

From 7-bromo-N4-methyl-quinazoline-2,4-diamine and 4-ethylsulfanyl-phenyl boronic acid: 7-(4-Ethylsulfanyl-phenyl)-N4-methyl-quinazoline-2,4-diamine trifluoroacetic acid salt as an off-white powder; (ES)⁺-HRMS m/e calcd for C₁₇H₁₈N₄S (M+H)⁺ 311.1325, found 311.1326.

### Example 69

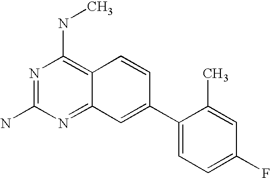

From 7-bromo-N4-methyl-quinazoline-2,4-diamine and 4-Fluoro-2-methyl-phenyl boronic acid: there was produced 7-(4-Fluoro-2-methyl-phenyl)-N4-methyl-quinazoline-2,4-diamine as a white powder; (ES)⁺-HRMS m/e calcd for C₁₆H₁₅FN₄ (M+H)⁺ 283.1354, found 283.1356.

### Example 70

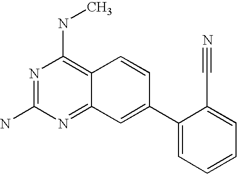

From 7-bromo-N4-methyl-quinazoline-2,4-diamine and 2-benzonitrile boronic acid: there was produced 2-(2-Amino-4-methylamino-quinazolin-7-yl)-benzonitrile as a light yellow solid; EI-HRMS m/e calcd for C₁₆H₁₃N₅ (M⁺) 275.1171, found 275.1170.

### Example 71

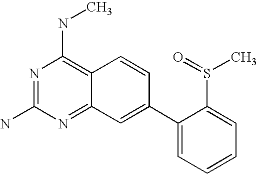

From oxidation of any conventional methods e.g. For general review of oxidation of thioethers to sulphoxide and sulphones, see Smith, M.; March, J.; Advanced Organic Chemistry, Wiley-Interscience: NY, 2001, pp. 1541-1542 and the references cited in it. Appropriate methods described in above reference of oxidation of an alkyl thio substituent to the corresponding sulfoxide group can be utilized to effect this conversion of N4-Methyl-7-(2-methylsulfanyl-phenyl)-quinazoline-2,4-diamine: there was produced 7-(2-Methanesulfinyl-phenyl)-N4-methyl-quinazoline-2,4-diamine as a white solid; (ES)⁺-HRMS m/e calcd for C₁₆H₁₆N₄OS (M+H)⁺ 313.1118, found 313.1121.

### Example 72

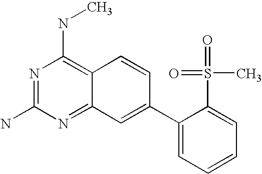

From oxidation of any conventional methods e.g. For general review of oxidation of thioethers to sulphoxide and sulphones, see Smith, M.; March, J.; Advanced Organic Chemistry, Wiley-Interscience: NY, 2001, pp. 1541-1542 and the references cited in it. Appropriate methods described in above reference of oxidation of an alkyl thio substituent to the corresponding sulfone group can be utilized to effect this conversion of 7-(2-Methanesulfinyl-phenyl)-N4-methyl-quinazoline-2,4-diamine: there was produced 7-(2-Methanesulfonyl-phenyl)-N4-methyl-quinazoline-2,4-diamine as a light yellow solid; (ES)⁺-HRMS m/e calcd for C₁₆H₁₆N₄O₂S (M+H)⁺ 329.1067, found 329.1070.

### Example 73

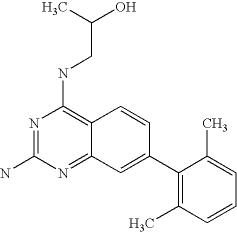

From 1-[2-Amino-7-bromo-quinazolin-4-ylamino]-propan-2-ol and 2,6 dimethyl-phenyl boronic acid: there was 2,6-dimethyl-phenyl)-quinazolin-4-ylamino]-propan-2-ol trifluoroacetic acid salt as an off-white solid; (ES)⁺-HRMS m/e calcd for C₁₉H₂₂N₄O (M+H)⁺ 323.1867, found 323.1869.

### Example 74

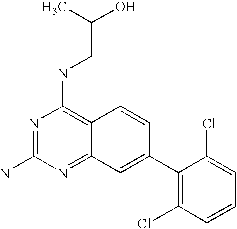

From 1-[2-Amino-7-bromo-quinazolin-4-ylamino]-propan-2-ol and 2, 6 dichloro-phenyl boronic acid: there was produced 1-[2-Amino-7-(2,6-dichloro-phenyl)-quinazolin-4-ylamino]-propan-2-ol trifluoroacetic acid salt as an off-white solid; (ES)⁺-HRMS m/e calcd for C₁₇H₁₆Cl₂N₄O (M+H)⁺ 363.0774, found 363.0777.

### Example 75

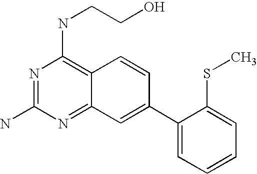

From 2-[2-Amino-7-bromo-quinazolin-4-ylamino]-ethanol and 2-methylsulfanyl-phenyl boronic acid: there was produced 2-[2-Amino-7-(2-methylsulfanyl-phenyl)-quinazolin-4-ylamino]-ethanol as an off-white solid; (ES)⁺-HRMS m/e calcd for C₁₇H₁₈N₄OS (M+H)⁺ 327.1274, found 327.1277.

### Example 76

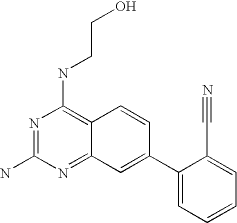

From 2-[2-Amino-7-bromo-quinazolin-4-ylamino]-ethanol and 2-benzonitrile boronic acid: there was produced 2-[2-Amino-4-(2-hydroxy-ethylamino)-quinazolin-7-yl]-benzonitrile as an off-white solid; (ES)⁺-HRMS m/e calcd for C₁₇H₁₅N₅O (M+H)⁺ 306.1350, found 306.1353.

### Example 77

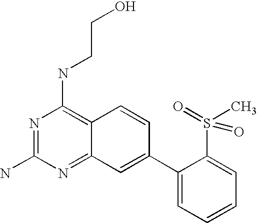

From oxidation of any conventional methods e.g. For general review of oxidation of thioethers to sulphoxide and sulphones, see Smith, M.; March, J.; Advanced Organic Chemistry, Wiley-Interscience: NY, 2001, pp. 1541-1542 and the references cited in it. Appropriate methods described in above reference of oxidation of an alkyl thio substituent to the corresponding sulfone group can be utilized to effect this conversion of 2-[2-Amino-7-(2-methanesulfinyl-phenyl)-quinazolin-4-ylamino]-ethanol (example 78): there was produced 2-[2-Amino-7-(2-methanesulfonyl-phenyl)-quinazolin-4-ylamino]-ethanol as an off-white solid; EI-HRMS m/e calcd for C₁₇H₁₈N₄O₃S (M⁺) 358.1100, found 358.1108.

### Example 78

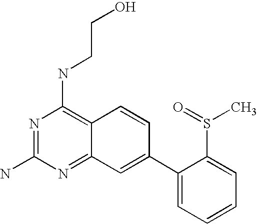

From oxidation of any conventional methods e.g. For general review of oxidation of thioethers to sulphoxide and sulphones, see Smith, M.; March, J.; Advanced Organic Chemistry, Wiley-Interscience: NY, 2001, pp. 1541-1542 and the references cited in it. Appropriate methods described in above reference of oxidation of an alkyl thio substituent to the corresponding sulphoxide group can be utilized to effect this conversion of 2-[2-Amino-7-(2-methylsulfanyl-phenyl)-quinazolin-4-ylamino]-ethanol (example 58): there was produced 2-[2-Amino-7-(2-methanesulfinyl-phenyl)-quinazolin-4-ylamino]-ethanol as an off-white solid; EI-HRMS m/e calcd for C₁₇H₁₈N₄O₂S (M⁺) 342.1150, found 342.1140.

### Example 79

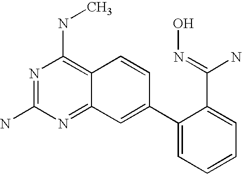

Cyano substituent can then be converted to cyano-hydroxyimino substituent. Any conventional method for converting a cyano substituent to cyano-hydroxyimino substituent can be utilized to effect this conversion as described in Hill, J., In Comprehensive Heterocyclic Chemistry, Vol. 6; Potts, K. T., Ed.; Pergamon: Oxford, 1984, 427 and the references cited in it. The above example was made from 2-[2-Amino-4-(2-hydroxy-ethylamino)-quinazolin-7-yl]-benzonitrile (example 70): there was produced 2-(2-Amino-4-methylamino-quinazolin-7-yl)-N-hydroxy-benzamidine as a white solid; EI-HRMS m/e calcd for C₁₆H₁₆N₆O (M−H)⁺ 307.1307, found 307.1305.

### Example 80

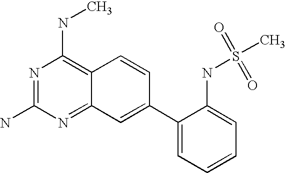

From 2-[2-Amino-7-bromo-quinazolin-4-ylamino]-ethanol and 2-methane sulfonamide phenyl boronic acid: there was produced N-[2-(2-Amino-4-methylamino-quinazolin-7-yl)-phenyl]-methanesulfonamide as a light yellow solid; EI-HRMS m/e calcd for C₁₆H₁₇N₅O₂S (M⁺) 343.1103, found 343.1111.

### Example 81

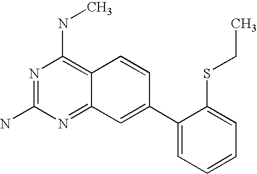

7-bromo-N4,N4-dimethyl-quinazoline-2,4-diamine From 2-[2-Amino-7-bromo-quinazolin-4-ylamino]-ethanol and 2-ethylsulfanyl-phenyl boronic acid: 7-(2-Ethylsulfanyl-phenyl)-N4-methyl-quinazoline-2,4-diamine as an off-white powder; EI-HRMS m/e calcd for C₁₇H₁₈N₄S (M⁺) 310.1252, found 310.1257.

### Example 82

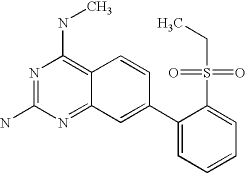

From oxidation of any conventional methods e.g. For general review of oxidation of thioethers to sulphoxide and sulphones, see Smith, M.; March, J.; Advanced Organic Chemistry, Wiley-Interscience: NY, 2001, pp. 1541-1542 and the references cited in it. Appropriate methods described in above reference of oxidation of an alkyl thio substituent to the corresponding sulfone group can be utilized to effect this conversion of 7-(2-Ethylsulfanyl-phenyl)-N4-methyl-quinazoline-2,4-diamine (example 81): there was produced 7-(2-Ethanesulfonyl-phenyl)-N4-methyl-quinazoline-2,4-diamine as a white solid; LRMS for C₁₇H₁₈N₄O₂S (M+H)⁺ at m/z=343.

### Example 83

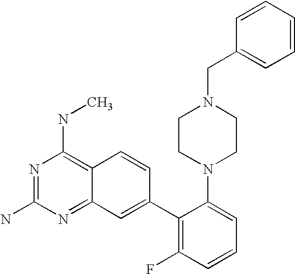

A mixture of 7-(2,6-difluorophenyl)-N4-methyl-quinazoline-2,4-diamine (example 23) (0.33 g, 1.153 mmol), anhydrous potassium carbonate (800.0 mg, 5.788 mmol), 1-benzylpiperazine (1.1 g, 6.241 mmol) and 1-methyl-2-pyrrolidinone (5 mL) was heated in a thick walled sealed tube at 200° C. for 3 days. The resultant reaction mixture was then cooled, diluted with water and extracted with 95:5:0.5 methylene chloride:methanol:aqueous ammonium hydroxide (3×100 mL)/ The combined organic layers were dried over anhydrous sodium sulfate, filtered, and concentrated. The crude material was purified by Biotage chromatography with 95:5:0.5 methylene chloride:methanol:aqueous ammonium hydroxide, followed by reverse phase HPLC with subsequent neutralization of the trifluoroacetic acid salt to the free base to obtain 7-[2-(4-Benzyl-piperazin-1-yl)-6-fluoro-phenyl]-N4-methyl-quinazoline-2,4-diamine (81.8 mg, 16.0%) as an off white solid. ¹H NMR (DMSO-d₆, 400 MHz) δ 7.95 (d, J=8.78 Hz, 1H), 7.91 (broad q, 1H), 7.27 (m, 7H), 7.10 (d, J=7.82 Hz, 1H), 6.92 (m, 2H), 6.16 (broad s, 2H), 3.34 (broad s, 2H), 2.96 (d, J=4.89 Hz, 3H), 2.77 (broad s, 4H), 2.19 (broad s, 4H).

### Example 84

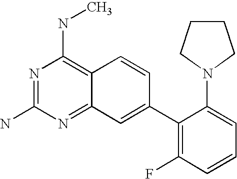

From 7-(2,6-Fluoro-phenyl)-N4-methyl-quinazoline-2,4-diamine (example 23) and pyrrolidine there was produced 7-(2-Fluoro-6-pyrrolidin-1-yl-phenyl)-N4-methyl-quinazoline-2,4-diamine trifluoroacetic acid salt as a light brown solid; EI-HRMS m/e calcd for C₁₉H₂₀FN₅ (M⁺) 337.1703, found 337.1691.

### Example 85

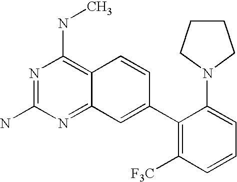

From 7-(2-Fluoro-6-trifluoromethyl-phenyl)-N4-methyl-quinazoline-2,4-diamine (example 136) and pyrrolidine, there was produced N4-Methyl-7-(2-pyrrolidin-1-yl-6-trifluoromethyl-phenyl)-quinazoline-2,4-diamine as a light brown powder, 70 mg; LRMS for C₂₀H₂₀F₃N₅ (M+H)⁺ at m/z=388.

### Example 86

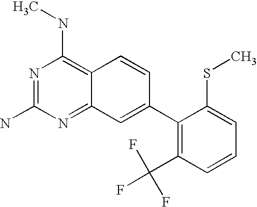

As described in scheme 6, A mixture of 7-bromo-N4-methyl-quinazoline-2,4-diamine (2.5 g, 9.878 mmol), tetrakis (triphenylphosphine) palladium(0) (2.44 g, 2.114 mmol) and hexa-n-butylditin (25.7 mL, 49.39 mmol) in N,N-dimethylacetamide (15 mL) was refluxed for 16 hours. The reaction suspension was passed through a celite pad and the resulting solution was added to water (10 mL) and extracted (3×100 mL) with methylene chloride:methanol:ammonium hydroxide (9:1:0.1). The combined organic layers were dried over anhydrous magnesium sulfate, filtered and concentrated. Flash column chromatography of the crude product was carried out with methylene chloride:methanol:ammonium hydroxide (90:10:1) to give N4-methyl-7-tributylstannanyl-quinazoline-2,4-diamine (2.6 g, 56.8%) as a light yellow oil. ¹H NMR (DMSO-d₆, 300 MHZ) δ 8.25 (broad s, 1H), 7.88 (d, J=8.06 Hz, 1H), 7.35 (s, 1H), 7.17 (d, J=8.05 Hz, 1H), 6.55 (broad s, 2H), 2.96 (d, J=4.39 Hz, 3H), 1.50 (m, 6H), 1.29 (m, 6H), 1.07 (m, 6H), 0.84 (t, J=7.15 Hz, 9H).

The coupling reaction can be carried out by a conventional aryl or heteroaromatic coupling partner utilizing Stille coupling. e.g. Stille et al., Angew. Chem. Int. Ed. Engl., 1986, 25, 508.

Typical conditions used to carry out the Stille reaction include the use of an organostannane as the coupling partner, palladium catalyst (2-20 mole %) such as tetrakis(triphenylphosphine)-palladium (0) or [1,1′bis(diphenylphosphino)-ferrocene]dichloro-palladium(II), a salt such as potassium fluoride or lithium chloride, in a suitable anhydrous solvent such as THF or DMF or ethylene glycol for at temperatures ranging from 25° C. to 125° C. for 2-18 hr

A mixture of N4-methyl-7-tributylstannanyl-quinazoline-2,4-diamine (800 mg, 1.727 mmol), 1-methylsulfanyl-2-bromo-3-trifluoromethyl-benzene prepared according to the general procedure detailed in scheme 5, (543.1 mg, 2.00 mmole), dichlorobis (triphenylphosphine) palladium(II) (242.4 mg, 0.345 mmol), tetrahydrofuran (2 mL) and N,N-dimethylformamide (2 mL) was placed in a thick walled vial, sealed and micro waved at 200° C. for one hour. The reaction mixture was then passed through an ion exchange pad and the resulting solution was added to water (10 mL) and extracted (3×100 mL) with methylene chloride:methanol:ammonium hydroxide (90:10:1). The combined organic layers were dried over anhydrous magnesium sulfate, filtered and concentrated. Two flash column chromatographies of the crude product were carried out with methylene chloride:methanol:ammonium hydroxide (90:10:1) obtained N4-methyl-7-(2-methylsulfanyl-6-trifluoromethyl-phenyl)-quinazoline-2,4-diamine (90 mg, 14.3%) as a white solid. ¹H NMR (DMSO-d₆, 400 MHz) δ 7.95 (m, 2H), 7.60 (broad s, 3H), 6.94 (s, 1H), 6.80 (d, J=8.79 Hz, 1H), 6.17 (broad s, 2H), 2.96 (d, J=3.91 Hz, 3H), 2.37 (s, 3H).

In an analogous manner, as described in scheme 6, there were obtained:

### Example 87

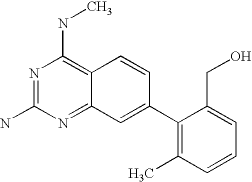

2-Iodo-3-methylbenzoic acid (2.6 g, 10 mmol) was dissolved in THF (15 mL), followed by slow addition of 1 M lithium aluminium hydride in THF (10 mL, 10 mmol). The colorless solution turned into light yellow. Upon the completion of addition, the reaction was stirred at ambient temperature for 30 min then quenched with water (50 mL). Ethyl acetate (25 mL) was added to the reaction mixture which was filtered and transferred to a separation funnel. The two layers were separated and the water layer was further extracted with ethyl acetate (2×100 mL). The combined organic layer was dried over sodium sulfate and concentrated to dryness to obtain 2-iodo-3-methylbenzyl alcohol as off-white solid (1.2 g); ¹H NMR (CDCl₃, 300 MHz) δ 1.92 (broad, 1H), 2.47 (s, 3H), 4.71 (s, 2H), 7.15-7.32 (m, 3H).

As described in Scheme 6, from N4-methyl-7-tributylstannanyl-quinazoline-2,4-diamine and 2-iodo-3-methylbenzyl alcohol: [2-(2-amino-4-methylamino-quinazolin-7-yl)-3-methyl-phenyl]-methanol as a white solid; LRMS for C₁₇H₁₈N₄O (M+H)⁺ at m/z=295.

### Example 88

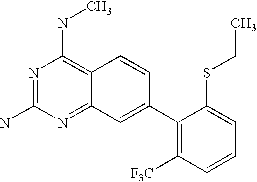

From N4-methyl-7-tributylstannanyl-quinazoline-2,4-diamine and 2-Ethylsulfanyl-6-trifluoromethyl-phenyl iodide prepared according to general procedure detailed in Scheme 6: there was produced 7-(2-Ethylsulfanyl-6-trifluoromethyl-phenyl)-N4-methyl-quinazoline-2,4-diamine as an off-white solid; EI-HRMS m/e calcd for C₁₈H₁₇F₃N₄S (M⁺) 378.1126, found 378.1134.

### Example 89

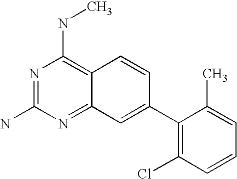

From N4-methyl-7-tributylstannanyl-quinazoline-2,4-diamine and 3-chloro-2-iodotoluene: 7-(2-Chloro-6-methyl-phenyl)-N4-methyl-quinazoline-2,4-diamine as an off-white solid; LRMS for C₁₆H₁₅ClN₄ (M+H)⁺ at m/z=299.

### Example 90

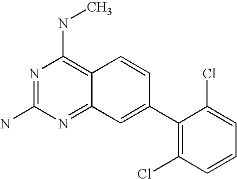

From N4-methyl-7-tributylstannanyl-quinazoline-2,4-diamine and 2,6-dichlorobenzoyl chloride: 7-(2,6-Dichloro-phenyl)-N4-methyl-quinazoline-2,4-diamine trifluoroacetic acid salt were obtained as an off white solid; LRMS for C₁₅H₁₂N₄Cl₂ (M+H)⁺ at m/z=320.

### Example 91

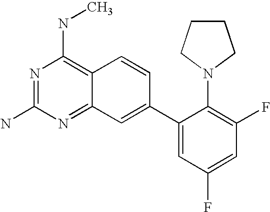

As described in Scheme 4, from 1-bromo-2,3,5-trifluorobenzene and pyrrolidine, there was produced 1-bromo-3,5-difluoro-2-(pyrrolidin-1-yl)benzene and this compound was used in the following synthesis.

As described in Scheme 6, by utilizing Stille coupling, from 1-bromo-3,5-difluoro-2-(pyrrolidin-1-yl)benzene and N4-methyl-7-tributylstannanyl-quinazoline-2,4-diamine: 7-(3,5-difluoro-2-pyrrolidin-1-yl-phenyl)-N4-methyl-quinazoline-2,4-diamine as a yellow solid; LRMS for C₁₉H₁₉F₂N₅ (M+H)⁺ at m/z=356.

### Example 92

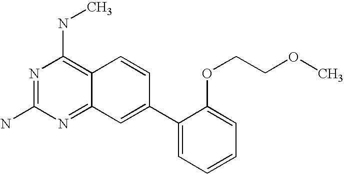

As described in Scheme 4, from 1-bromo-2-fluorobenzene and 2-ethoxyethanol, there was produced 1-bromo-2-(2-methoxy-ethoxy)benzene: ¹H NMR □ (DMSO-d₆, 300 MHz) δ 3.32 (s, 3H), 3.67 (m, 2H), 4.14 (m, 2H), 6.87 (ddd, J₁=7.3 Hz, J₂=7.8 Hz, J₃=1.5 Hz, 1H), 7.09 (dd, J₁=8.2 Hz, J₂=1.5 Hz, 1H), 7.31 (ddd, J₁=7.3 Hz, J₂=8.2 Hz, J₃=1.6 Hz, 1H), 7.55 (dd, J₁=7.8 Hz, J₂=1.6 Hz, 1H).

As described in Scheme 6, by utilizing Stille coupling from 1-bromo-2-(2-methoxy-ethoxy)benzene and N4-methyl-7-tributylstannanyl-quinazoline-2,4-diamine: 7-[2-(2-methoxy-ethoxy)-phenyl]-N4-methyl-quinazoline-2,4-diamine as a white semisolid; LRMS for C₁₈H₂₀N₄O₂ (M+H)⁺ at m/z=325.

### Example 93

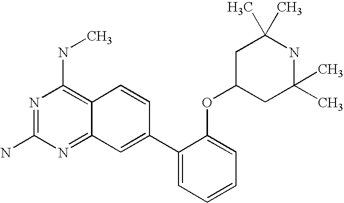

As described in Scheme 4, from 1-bromo-2-fluorobenzene and 2,2,6,6-tetramethyl-4-hydroxypiperidine, there was produced 1-bromo-2-(2,2,6,6-tetramethyl-piperidin-4-yloxy)benzene: ¹H NMR (DMSO-d₆, 300 MHz) δ1.07 (s, 6H), 1.15 (s, 6H), 1.13-1.26 (m, 2H), 1.90 (m, 2H), 4.79 (m, 1H), 6.85 (m, 1H), 7.15 (m, 1H), 7.31 (m, 1H), 7.54 (dd, J₁=8.0 Hz, J₂=1.6 Hz, 1H).

As described in Scheme 6, by utilizing Stille coupling from 1-bromo-2-(2,2,6,6-tetramethyl-piperidin-4-yloxy)benzene and N4-methyl-7-tributylstannanyl-quinazoline-2,4-diamine: N4-methyl-7-[2-(2,2,6,6-tetramethyl-piperidin-4-yloxy)-phenyl]-quinazoline-2,4-diamine trifluoroacetic acid salt as a light yellow solid; LRMS for C₂₄H₃₁N₅O (M+H)⁺ at m/z=406.

### Example 94

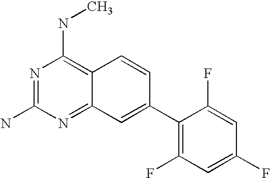

By utilizing Stille coupling, from 1-bromo-2,4,6-triflorobenzene and N4-methyl-7-tributylstannanyl-quinazoline-2,4-diamine:N4-Methyl-7-(2,4,6-trifluoro-phenyl)-quinazoline-2,4-diamine trifluoroacetic acid salt as a light yellow solid; LRMS for C₁₅H₁₁F₃N₄ (M+H)⁺ at m/z=305.

### Example 95

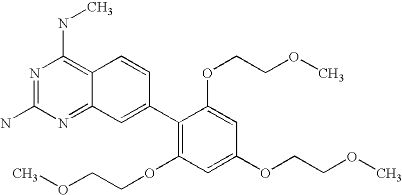

As described in Scheme 4, from 1-bromo-2,4,6-trifluorobenzene and 2-methoxyethanol, there was produced 1-bromo-2,4,6-tri(2-methoxy-ethoxy)benzene and this compound was used in the in next step.

As described in Scheme 6, By utilizing Stille coupling from 1-bromo-2,4,6-tri(2-methoxy-ethoxy)benzene and N4-methyl-7-tributylstannanyl-quinazoline-2,4-diamine: N4-methyl-7-[2,4,6-tris-(2-methoxy-ethoxy)-phenyl]-quinazoline-2,4-diamine trifluoroacetic acid salt as a light yellow solid; LRMS for C₂₄H₃₂N₄O₆ (M+H)⁺ at m/z=473.

By utilizing Stille coupling, from 1-bromo-2,4,6-tri(2-methoxy-ethoxy)benzene and N4-methyl-7-tributylstannanyl-quinazoline-2,4-diamine:N4-Methyl-7-[2,4,6-tris-(2-methoxy-ethoxy)-phenyl]-quinazoline-2,4-diamine trifluoroacetic acid salt as a light yellow solid; LRMS for C₂₄H₃₂N₄O₆ (M+H)⁺ at m/z=473.

### Example 96

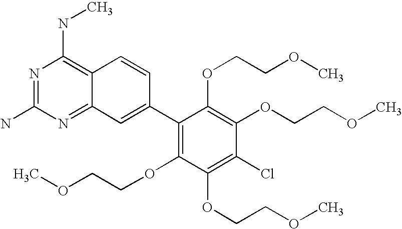

As described in Scheme 4, from 1-bromo-4-chloro-2,3,5,6-tetrafluorobenzene and 2-methoxyethanol, there was produced 1-bromo-4-chloro-2,3,5,6-tetrakis(2-methoxy-ethoxy)benzene and this compound was used in the following synthesis.

As described in Scheme 6, from 1-bromo-4-chloro-2,3,5,6-tetrakis(2-methoxy-ethoxy)benzene and N4-methyl-7-tributylstannanyl-quinazoline-2,4-diamine: 7-[4-chloro-2,3,5,6-tetrakis-(2-methoxy-ethoxy)-phenyl]-N4-methyl-quinazoline-2,4-diamine trifluoroacetic acid salt as a light yellow oil; LRMS for C₂₇H₃₇ClN₄O₈ (M+H)⁺ at m/z=581.

### Example 97

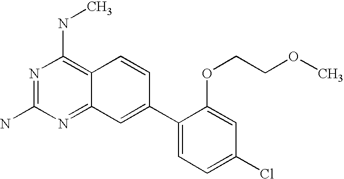

As described in Scheme 4, from 4-chloro-2-fluoro-1-iodoobenzene and 2-methoxyethanol, there was produced 4-chloro-1-iodo-2-(2-methoxy-ethoxy)benzene: LRMS for C₉H₁₀ClBrO₂ (M+H)⁺ at m/z=313.

As described in Scheme 6, from 4-chloro-1-iodo-2-(2-methoxy-ethoxy)benzene and N4-methyl-7-tributylstannanyl-quinazoline-2,4-diamine: 7-[4-chloro-2-(2-methoxy-ethoxy)-phenyl]-N4-methyl-quinazoline-2,4-diamine as a white solid; LRMS for C₁₈H₁₉ClN₄O₂ (M+H)⁺ at m/z=359.

### Example 98

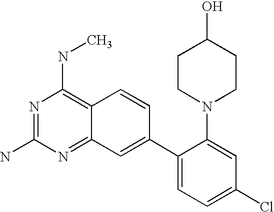

As described in Scheme 4, from 4-chloro-2-fluoro-1-iodoobenzene and 4-hydroxypiperidine, there was produced (4-chloro-2-iodo-phenyl)piperidin-4-ol: LRMS for C₁₁H₁₃ClBrNO (M+H)⁺ at m/z=338.

As described in Scheme 6, from (4-chloro-2-iodo-phenyl)piperidin-4-ol and N4-methyl-7-tributylstannanyl-quinazoline-2,4-diamine: 1-[2-(2-Amino-4-methylamino-quinazolin-7-yl)-5-chloro-phenyl]-piperidin-4-ol as a light yellow solid; LRMS for C₂₀H₂₂ClN₅O (M+H)⁺ at m/z=384.

### Example 99

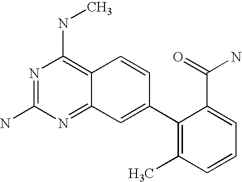

2-Iodo-3-methylbenzoic acid (2.0 g, 7.6 mmol), EDCl (2.2 g, 11.45 mmol), HOBt (1.74 g, 11.45 mmol), DIEA (5.3 ml, 30.4 mmol) and ammonium chloride (0.813 g, 15.2 mmol) were dissolved in DMF (14 mL) and the mixture was stirred at ambient temperature for 2 h. Water (50 ml) was added to the reaction mixture and thus formed mixture was stirred for 10 min. The white precipitate was collected, washed with water and diethyl ether and dried. 2-Iodo-3-methylbenzamide (1.80 g) was obtained as a white solid; ¹H NMR (DMSO-d₆, 300 MHz) δ 2.41 (s, 3H), 7.06 (m, 1H), 7.23-7.37 (m, 2H), 7.47 (broad, 1H), 7.76 (broad, 1H).

As described in Scheme 6, from 2-Iodo-3-methyl-benzamide and N4-methyl-7-tributylstannanyl-quinazoline-2,4-diamine: 2-(2-amino-4-methylamino-quinazolin-7-yl)-3-methyl-benzamide as an off-white powder; LRMS for C₁₇H₁₇N₅₀ (M+H)⁺ at m/z=308.

### Example 100

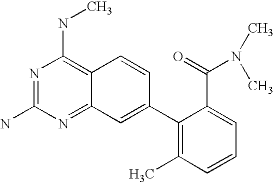

From a coupling reaction of 2-iodo-3-methylbenzoic acid and N,N-dimethylamine, there was produced 2-iodo-3,N,N-trimethylbenzamide: ¹H NMR (DMSO-d₆, 300 MHz) δ 2.41 (s, 3H), 2.72 (m, 3H), 2.98 (m, 3H), 6.98 (m, 1H), 7.32 (m, 2H).

As described in Scheme 6, from 2-iodo-3,N,N-trimethylbenzamide and N4-methyl-7-tributylstannanyl-quinazoline-2,4-diamine: 2-(2-amino-4-methylamino-quinazolin-7-yl)-3,N,N-trimethyl-benzamide as an off-white powder; LRMS for C₁₉H₂₁N₅O (M+H)⁺ at m/z=336.

### Example 101

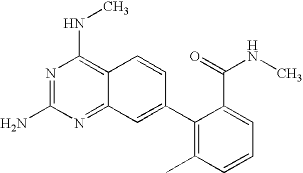

2-iodo-3,N-dimethylbenzamide was obtained from 2-iodo-3-methylbenzoic acid and methylamine; ¹H NMR (DMSO-d₆, 300 MHz) δ 2.41 (s, 3H), 2.72 (m, 3H), 7.03 (m, 1H), 7.31 (m, 2H), 8.23 (s, 1H). As described in Scheme 6, from 2-iodo-3,N-dimethylbenzamide and N4-methyl-7-tributylstannanyl-quinazoline-2,4-diamine: 2-(2-amino-4-methylamino-quinazolin-7-yl)-3,N-dimethyl-benzamide as a white powder; LRMS for C₁₈H₁₉N₅O (M+H)⁺ at m/z=322.

### Example 102

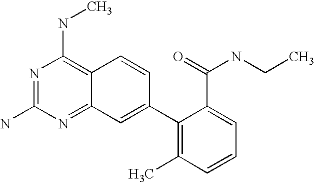

N-Ethyl-2-iodo-3-methylbenzamide was obtained from the coupling reaction of 2-iodo-3-methylbenzoic acid and ethylamine in THF solution. LRMS for C₁₀H₁₂NOBr (M+H)⁺ at m/z=289.

As described in Scheme 6, from N-ethyl-2-iodo-3-methylbenzamide and N4-methyl-7-tributylstannanyl-quinazoline-2,4-diamine: 2-(2-amino-4-methylamino-quinazolin-7-yl)-N-ethyl-3-methyl-benzamide as a white powder; LRMS for C₁₉H₂₁N₅O (M+H)⁺ at m/z=336.

### Example 103

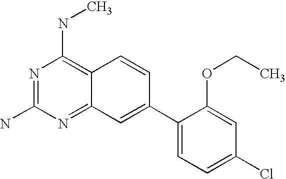

As described in Scheme 4, from 4-chloro-2-fluoro-iodobenzene and ethanol, there was produced 4-choloro-2-ethoxy-iodobenzene and this compound was used in the following synthesis.

As described in Scheme 6, from 4-choloro-2-ethoxy-iodobenzene and N4-methyl-7-tributylstannanyl-quinazoline-2,4-diamine: 7-(4-chloro-2-ethoxy-phenyl)-N4-methyl-quinazoline-2,4-diamine as a light yellow solid; LRMS for C₁₇H₁₇ClN₄O (M+H)⁺ at m/z=329.

### Example 104

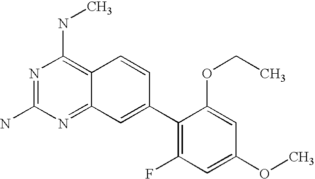

As described in Scheme 4, from 2,6-difluoro-4-methoxy-bromobenzene and ethanol, there was produced 2-ethoxy-6-fluoro-4-methoxy-bromobenzene and this compound was used in the following synthesis.

As described in Scheme 6, from 2-ethoxy-6-fluoro-4-methoxy-bromobenzene and N4-methyl-7-tributylstannanyl-quinazoline-2,4-diamine: 7-(2-ethoxy-6-fluoro-4-methoxy-phenyl)-N4-methyl-quinazoline-2,4-diamine as a white powder; LRMS for C₁₈H₁₉FN₄O₂ (M+H)⁺ at m/z=343.

### Example 105

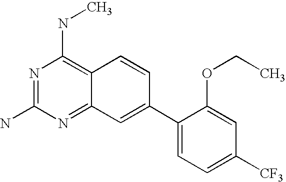

As described in Scheme 4, from 2-fluoro-4-trifluoromethyl-bromobenzene and ethanol, there was produced 2-ethoxy-4-trifluoromethyl-bromobenzene and this compound was used in the following synthesis.

As described in Scheme 6, from 2-ethoxy-4-trifluoromethyl-bromobenzene and N4-methyl-7-tributylstannanyl-quinazoline-2,4-diamine: 7-(2-ethoxy-4-trifluoromethyl-phenyl)-N4-methyl-quinazoline-2,4-diamine trifluoroacetic acid salt as a white powder; LRMS for C₁₈H₁₇F₃N₄O (M+H)⁺ at m/z=363.

### Example 106

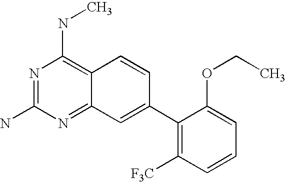

As described in Scheme 4, from 2-fluoro-6-trifluoromethyl-bromobenzene and ethanol, there was produced 2-ethoxy-6-trifluoromethyl-bromobenzene and this compound was used in the following synthesis.

As described in Scheme 6, from 2-ethoxy-6-trifluoromethyl-bromobenzene and N4-methyl-7-tributylstannanyl-quinazoline-2,4-diamine: 7-(2-ethoxy-6-trifluoromethyl-phenyl)-N4-methyl-quinazoline-2,4-diamine trifluoroacetic acid salt as a white powder; LRMS for C₁₈H₁₇F₃N₄O (M+H)⁺ at m/z=363.

### Example 107

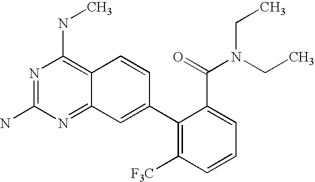

From the coupling reaction of 2-(2-amino-4-methylamino-quinazolin-7-yl)-3-methyl-benzoic acid and N,N-diethylamine: 2-(2-Amino-4-methylamino-quinazolin-7-yl)-N,N-diethyl-3-methyl-benzamide as an off-white powder; LRMS for C₂₁H₂₅N₅O (M+H)⁺ at m/z=364.

### Example 108

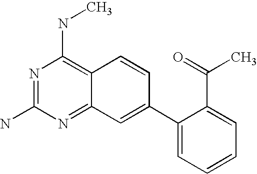

As described in Scheme 6, from 1-bromo-acetophenone and N4-methyl-7-tributylstannanyl-quinazoline-2,4-diamine: 1-[2-(2-Amino-4-methylamino-quinazolin-7-yl)-phenyl]-ethanone as a light yellow powder; LRMS for C₁₇H₁₆N₄O (M+H)⁺ at m/z=293.

### Example 109

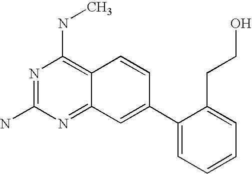

As described in Scheme 6, from 2-(2-bromophenyl)ethanol and N4-methyl-7-tributylstannanyl-quinazoline-2,4-diamine: 2-[2-(2-Amino-4-methylamino-quinazolin-7-yl)-phenyl]-ethanol as an off-white powder; LRMS for C₁₇H₁₈N₄O (M+H)⁺ at m/z=295.

### Example 110

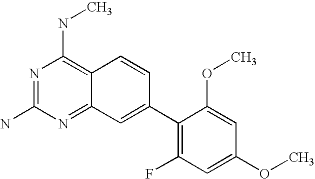

As described in Scheme 6, from 2,4-dimethoxy-6-fluoro-bromobenzene and N4-methyl-7-tributylstannanyl-quinazoline-2,4-diamine: 7-(2-fluoro-4,6-dimethoxy-phenyl)-N4-methyl-quinazoline-2,4-diamine as a white powder; LRMS for C₁₇H₁₇FN₄O₂ (M+H)⁺ at m/z=329.

### Example 111

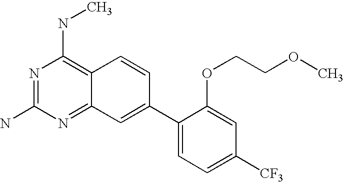

As described in Scheme 4, from 2-fluoro-6-trifluoromethyl-bromobenzene and 2-methoxyethanol, there were produced 2-(2-methoxy-ethoxy)-4-trifluoromethyl-bromobenzene and this compound was used in the following synthesis.

As described in Scheme 6, from 2-(2-methoxy-ethoxy)-4-trifluoromethylbromobenzene and N4-methyl-7-tributylstannanyl-quinazoline-2,4-diamine: 7-[2-(2-methoxy-ethoxy)-4-trifluoromethyl-phenyl]-N4-methyl-quinazoline-2,4-diamine as a white powder; LRMS for C₁₉H₁₉F₃N₄O₂ (M+H)⁺ at m/z=393.

### Example 112

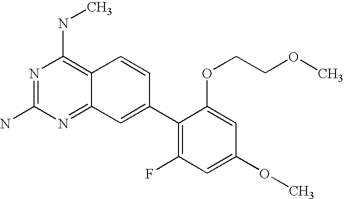

As described in Scheme 4, from 2,6-difluoro-4-methoxy-bromobenzene and 2-methoxyethanol, there was prepared 6-fluoro-4-methoxy-2-(2-methoxy-ethoxy)-bromobenzene and this compound was used in the following synthesis.

As described in Scheme 6, from 6-fluoro-4-methoxy-2-(2-methoxy-ethoxy)-bromobenzene and N4-methyl-7-tributylstannanyl-quinazoline-2,4-diamine: 7-[6-fluoro-4-methoxy-2-(2-methoxy-ethoxy)-phenyl]-N4-methyl-quinazoline-2,4-diamine trifluoroacetic acid salt as a white powder; LRMS for C₁₉H₂₁FN₄O₃ (M+H)⁺ at m/z=373.

### Example 113

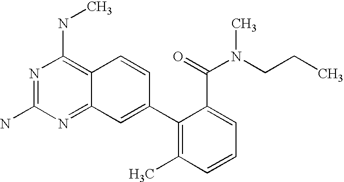

From the coupling reaction of 2-iodo-3-methylbenzoic acid and N-methyl-N-propylamine, there was produced 2-iodo-3,N-dimethyl-N-propylbenamide as a light yellow oil; LRMS for C₁₂H₁₆NOI (M+H)⁺ at m/z=318.

As described in Scheme 6, from 2-iodo-3,N-dimethyl-N-propylbenamide and N4-methyl-7-tributylstannanyl-quinazoline-2,4-diamine: 2-(2-amino-4-methylamino-quinazolin-7-yl)-3,N-dimethyl-N-propyl-benzamide as an off-white powder; LRMS for C₂₁H₂₅N₅O (M+H)⁺ at m/z=364.

### Example 114

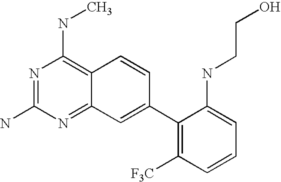

As described in Scheme 4, from 2-fluoro-6-trifluoromethyl-bromobenzene and 2-aminoethanol, there were produced 2-(2-hydroxy-ethylamino)-6-trifluoromethyl-bromobenzene and this compound was used in the following synthesis.

As described in Scheme 6, from 2-(2-hydroxy-ethylamino)-6-trifluoromethyl-bromobenzene and N4-methyl-7-tributylstannanyl-quinazoline-2,4-diamine: 2-[2-(2-amino-4-methylamino-quinazolin-7-yl)-3-trifluoromethyl-phenylamino]-ethanol as a light yellow powder; LRMS for C₁₈H₁₈F₃N₅O (M+H)⁺ at m/z=378.

### Example 115

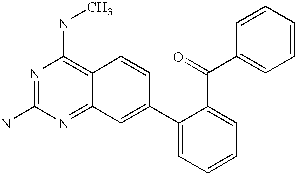

As described in Scheme 6, from (2-bromo-phenyl)-phenyl-methanone and N4-methyl-7-tributylstannanyl-quinazoline-2,4-diamine: [2-(2-amino-4-methylamino-quinazolin-7-yl)-phenyl]-phenyl-methanone as a light yellow powder; LRMS for C₂₂H₁₈N₄O (M+H)⁺ at m/z=355.

### Example 116

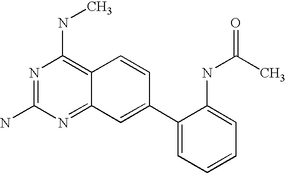

As described in Scheme 6, from N-(2-Bromo-phenyl)-acetamide and N4-methyl-7-tributylstannanyl-quinazoline-2,4-diamine: N-[2-(2-Amino-4-methylamino-quinazolin-7-yl)-phenyl]-acetamide as a light yellow powder; LRMS for C₁₇H₁₇N₅O (M+H)⁺ at m/z=308.

### Example 117

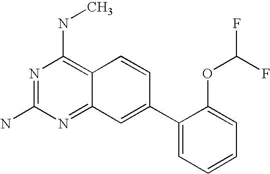

As described in Scheme 6, from 1-bromo-2-difluoromethoxy-benzene and N4-methyl-7-tributylstannanyl-quinazoline-2,4-diamine: 7-(2-difluoromethoxy-phenyl)-N4-methyl-quinazoline-2,4-diamine as a light yellow powder; LRMS for C₁₆H₁₄F₂N₄O (M+H)⁺ at m/z=317.

### Example 118

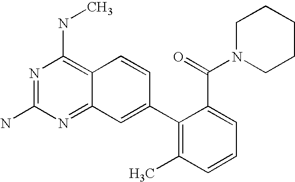

From the coupling reaction of 2-iodo-3-methylbenzoic acid and piperidine, there was produced (2-iodo-3-methyl-phenyl)-piperidin-1-yl-methanone and this compound was used in the following synthesis.

As described in Scheme 6, from (2-iodo-3-methyl-phenyl)-piperidin-1-yl-methanone and N4-methyl-7-tributylstannanyl-quinazoline-2,4-diamine: [2-(2-amino-4-methylamino-quinazolin-7-yl)-3-methyl-phenyl]-piperidin-1-yl-methanone as an off-white powder; LRMS for C₂₂H₂₅N₅O (M+H)⁺ at m/z=376.

### Example 119

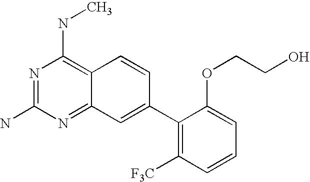

As described in Scheme 4, from 2-fluoro-6-trifluoromethyl-bromobenzene and 2-hydroxyethanol, there was prepared 2-(2-hydroxy-ethoxy)-6-trifluoromethylbromobenzene and this compound was used in the following synthesis.

As described in Scheme 6, from 2-(2-hydroxy-ethoxy)-6-trifluoromethylbromobenzene and N4-methyl-7-tributylstannanyl-quinazoline-2,4-diamine: 2-[2-(2-amino-4-methylamino-quinazolin-7-yl)-3-trifluoromethyl-phenoxy]-ethanol as a white powder; LRMS for C₁₈H₁₇F₃N₄O₂ (M+H)⁺ at m/z=379.

### Example 120

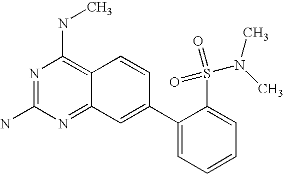

Preparation of 2-bromo-N,N-dimethyl-benzenesulfonamide. 2-Bromo-benzenesulfonyl chloride (1 g, 3.91 mmol) was mixed with N,N-dimethylamine (7.8 mL) in THF and followed by addition of pyridine (5 mL) and DMF (3 mL). The mixture was stirred at ambient temperature for 2 h. Water (5 mL) was added to the mixture and the mixture was extracted with ethyl acetate (3×20 mL). The combined organic layer was washed with diluted brine and brine. 2-Bromo-N,N-dimethyl-benzenesulfonamide was obtained as light yellow solid (0.893 g); LRMS for C₈H₁₀NO₂Br (M+H)⁺ at m/z=265

As described in Scheme 6, from 2-bromo-N,N-dimethyl-benzenesulfonamide and N4-methyl-7-tributylstannanyl-quinazoline-2,4-diamine:2-(2-aAmino-4-methylamino-quinazolin-7-yl)-N,N-dimethyl-benzenesulfonamide as a white solid; LRMS for C₁₇H₁₉N₅O₂S (M+H)⁺ at m/z=358.

### Example 121

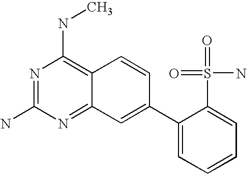

As described in Scheme 6, from 2-bromo-benzenesulfonamide and N4-methyl-7-tributylstannanyl-quinazoline-2,4-diamine: 2-(2-amino-4-methylamino-quinazolin-7-yl)-benzenesulfonamide as a light yellow solid; LRMS for C₁₅H₁₅N₅O₂S (M+H)⁺ at m/z=330.

### Example 122

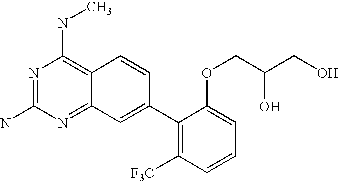

As described in Scheme 4, from 2,2-dimethyl-1,3-dioxolane-4-methanol and 2-fluoro-6-trifluorobromobenzene: 4-[(2-bromo-3-trifluoromethyl-phenoxy)methyl]-2,2-dimethyl-1,3-dioxolane; ¹H NMR (DMSO-d₆, 300 MHz) δ 1.41 (s, 3H,), 1.48 (s, 3H), 4.01-4.26 (m, 4H), 4.52 (m, 1H), 7.01 (dd, J₁=7.6 Hz, J₂=1.9 Hz, 1H), 7.30-7.40 (m, 2H).

As described in Scheme 6, from 4-[(2-bromo-3-trifluoromethyl-phenoxy)methyl]-2,2-dimethyl-1,3-dioxolane and N4-methyl-7-tributylstannanyl-quinazoline-2,4-diamine: 3-[2-(2-Amino-4-methylamino-quinazolin-7-yl)-3-trifluoromethyl-phenoxy]-propane-1,2-diol as an off-white solid; LRMS for C₁₉H₁₉F₃N₄O₃ (M+H)⁺ at m/z=409.

### Example 123

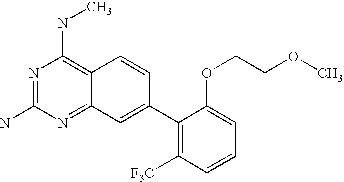

From 2-methoxyethanol and 7-(2-fluoro-6-trifluoromethyl-phenyl)-N4-methyl-quinazoline-2,4-diamine (example 136), 172 mg, 0.512 mmol) was added. The resulted mixture was heated to 125° C. in a pressure tube for 16 h. After concentration, the residue was applied on silica gel chromatography and the isolated product was further purified by reverse phase HPLC. The purified product was passed through an ion exchange column and 7-[2-(2-Methoxy-ethoxy)-6-trifluoromethyl-phenyl]-N4-methyl-quinazoline-2,4-diamine was obtained as an off-white solid (65.9 mg); LRMS for C₁₉H₁₉F₃N₄O₂ (M+H)⁺ at m/z=393.

### Example 124

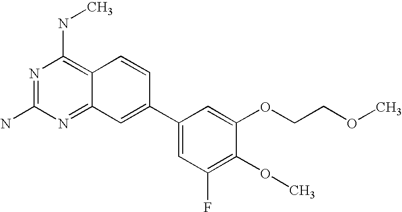

As described in Scheme 4, from 3,5-difluoro-4-methoxy-bromobenzene and 2-methoxyethanol, there was produced 5-fluoro-4-methoxy-3-(2-methoxy-ethoxy)-bromobenzene and this compound was used in the following synthesis.

As described in Scheme 6, from 5-fluoro-4-methoxy-3-(2-methoxy-ethoxy)-bromobenzene and N4-methyl-7-tributylstannanyl-quinazoline-2,4-diamine: 7-[5-fluoro-4-methoxy-3-(2-methoxy-ethoxy)-phenyl]-N4-methyl-quinazoline-2,4-diamine as a white solid; LRMS for C₁₉H₂₁FN₄O₃ (M+H)⁺ at m/z=373.

### Example 125

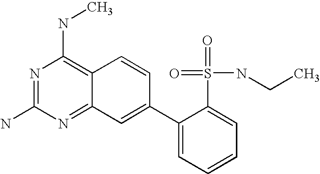

2-Bromosulfonyl chloride (2.0 g, 7.83 mmol) was dissolved in anhydrous DMF (5 mL) and anhydrous pyridine (5 mL), followed by addition of ethylamine in THF (2 M solution, 7.83 mL, 15.66 mmol). The so formed solution was stirred at ambient temperature for 4 h. Ethyl acetate and 1 N HCl were added to the mixture. The organic layer was washed with brine and 2 M Na₂CO₃ and dried over MgSO₄. After concentration of the organic layer, 2-bromo-N-ethyl-benzenesulfonamide (0.51 g) was obtained as a brown solid; LRMS for C₈H₁₀BrSNO₂ (M+H)⁺ at m/z=265.

As described in Scheme 6, from 2-bromo-N-ethyl-benzenesulfonamide and N4-methyl-7-tributylstannanyl-quinazoline-2,4-diamine: 2-(2-Amino-4-methylamino-quinazolin-7-yl)-benzenesulfonamide was obtained as a light yellow solid; LRMS for C₁₇H₁₉N₅O₂S (M+H)⁺ at m/z=358.

### Example 126

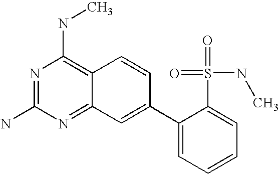

2-Bromosulfonyl chloride (2.0 g, 7.83 mmol) was dissolved in anhydrous DMF (5 mL) and anhydrous pyridine (5 mL), followed by addition of methylamine in THF (2 M solution, 7.83 mL, 15.66 mmol). The so formed solution was stirred at ambient temperature for 4 h. Ethyl acetate and 1 N HCl were added to the mixture. The organic layer was washed with brine and 2 M Na₂CO₃ and dried over MgSO₄. After concentration of the organic layer, 2-bromo-N-methyl-benzenesulfonamide (1.70 g) was obtained as a yellow solid; ¹H NMR (CDCl₃, 300 MHz) δ 2.62 (d, J=5.4 Hz, 3H), 5.25 (broad, 1H), 7.47 (m, 2H), 7.75 (dd, J₁=7.6 Hz, J₂=1.5 Hz, 1H), 8.14 (dd, J₁=7.5 Hz, J₂=2.0 Hz, 1H).

As described in Scheme 6, from 2-bromo-N-methyl-benzenesulfonamide and N4-methyl-7-tributylstannanyl-quinazoline-2,4-diamine: N-methyl-2-(2-amino-4-methylamino-quinazolin-7-yl)-benzenesulfonamide was obtained as a light white solid; m.p.=316-317° C.; LRMS for C₁₆H₁₇N₅O₂S (M+H)⁺ at m/z=344.

### Example 127

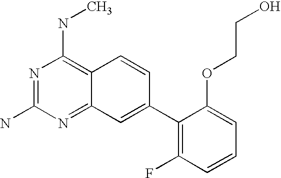

As described in Scheme 4, from 2,6-difluoro-bromobenzene and ethylene glycol, there was produced 6-fluoro-2-(2-hydroxy-ethoxy)-bromobenzene and this compound was used in the following synthesis.

As described in Scheme 6, from 6-fluoro-2-(2-hydroxy-ethoxy)-bromobenzene and N4-methyl-7-tributylstannanyl-quinazoline-2,4-diamine: 7-[6-fluoro-2-(2-hydroxy-ethoxy)-phenyl]-N4-methyl-quinazoline-2,4-diamine as a white solid; LRMS for C₁₇H₁₇FN₄O₂ (M+H)⁺ at m/z=329.

### Example 128

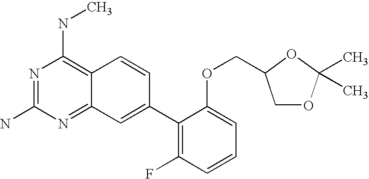

As described in Scheme 4, from 2,6-difluoro-bromobenzene and 2,2-dimethyl-1,3-dioxolane-4-methanol, there was produced 2-(2,2-dimethyl-[1,3]dioxolan-4-yl-methoxy)-6-fluoro-bromobenzene and this compound was used in the following synthesis.

As described in Scheme 6, from 2-(2,2-dimethyl-[1,3]dioxolan-4-ylmethoxy)-6-fluoro-bromobenzene and N4-methyl-7-tributylstannanyl-quinazoline-2,4-diamine: 7-[2-(2,2-dimethyl-[1,3]dioxolan-4-ylmethoxy)-6-fluoro-phenyl]-N4-methyl-quinazoline-2,4-diamine hydrochloride salt as a white solid; LRMS for C₂₁H₂₃FN₄O₃ (M+H)⁺ at m/z=399.

### Example 129

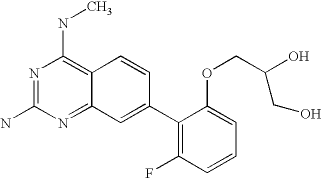

As described in Scheme 4, from 2,6-difluoro-bromobenzene and 2,2-dimethyl-1,3-dioxolane-4-methanol, there was prepared 3-(2-bromo-3-fluoro-phenoxy)-propane-1,2-diol and this compound was used in the following synthesis.

As described in Scheme 6, from 3-(2-bromo-3-fluoro-phenoxy)-propane-1,2-diol and N4-methyl-7-tributylstannanyl-quinazoline-2,4-diamine: 3-[2-(2-amino-4-methylamino-quinazolin-7-yl)-3-fluoro-phenoxy]-propane-1,2-diol as a light yellow solid; LRMS for C₁₈H₁₉FN₄O₃ (M+H)⁺ at m/z=359.

### Example 130

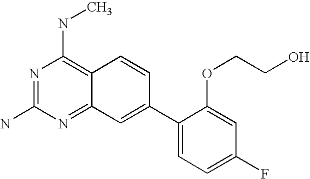

Preparation of (2-bromo-5-fluoro-phenoxy)-acetic acid methyl ester. 2-Bromo-5-fluoro-phenol (2.0 g, 10.47 mmol), methyl 2-bromoacetate (1.2 mL, 12.56 mmol) and potassium carbonate (4.3 g, 31.41 mmol) were suspended in DMF (20 mL). The mixture was heated to 68° C. for 18 h and 90° C. for 4 h. Water and brine were added to the reaction mixture. The mixture was extracted with ethyl acetate twice. The combined organic layer was washed with dilute brine and brine, dried over sodium sulfate and concentrated in vacuo. The residue was purified on silica gel flash chromatography. (2-Bromo-5-fluoro-phenoxy)-acetic acid methyl ester was obtained as an off-white solid; ¹H NMR (CDCl₃, 300 MHz) δ 3.82 (s, 3H), 4.70 (s, 2H), 6.55 (dd, J₁=10.0 Hz, J₂=2.7 Hz, 1H), 6.64 (dt, J₁=8.7 Hz, J2=8.7 Hz, J₃=2.7 Hz, 1H), 7.50 (dd, J₁=8.7 Hz, J₂=6.1 Hz, 1H).

As described in Scheme 5, from (2-bromo-5-fluoro-phenoxy)-acetic acid methyl ester and N4-methyl-7-tributylstannanyl-quinazoline-2,4-diamine: [2-(2-amino-4-methylamino-quinazolin-7-yl)-5-fluoro-phenoxy]-acetic acid was obtained as an yellow oil (0.32 g).

[2-(2-Amino-4-methylamino-quinazolin-7-yl)-5-fluoro-phenoxy]-acetic acid (0.32 g, 0.93 mmol) was dissolved in a mixture of THF and dioxane, followed by addition of excess amount of lithium aluminum hydride. The resulted mixture was heated to 60° C. for 16 h. The reaction was cooled, quenched with methanol and concentrated. The residue was suspended in water and adjusted to pH=7. The resulted mixture was extracted with ethyl acetate (95%) and methanol (5%). The organic layer was washed with brine, dried and concentrated. The residue was dissolved in methanol (10%) and dichloromethane (90%), loaded onto an ion exchange column and eluted with 1.7 N ammonium in methanol and dichloromethane. Upon concentration, the residue was purified by reverse phase HPLC. 2-[2-(2-Amino-4-methylamino-quinazolin-7-yl)-5-fluoro-phenoxy]-ethanol was obtained (7.4 mg) as an off-white power; LRMS for C₁₇H₁₇FN₄O₂ (M+H)⁺ at m/z=329.

### Example 131

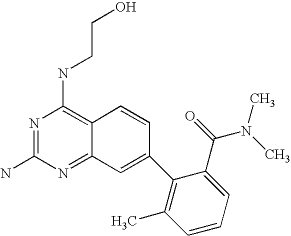

From a coupling reaction of 2-iodo-3-methylbenzoic acid and N,N-dimethylamine, there was produced 2-iodo-3,N,N-trimethylbenzamide: ¹H NMR (DMSO-d₆, 300 MHz) δ 2.41 (s, 3H), 2.72 (m, 3H), 2.98 (m, 3H), 6.98 (m, 1H), 7.32 (m, 2H).

As described in Scheme 6, from 2-iodo-3,N,N-trimethylbenzamide and 2-(2-Amino-7-tributylstannanyl-quinazolin-4-ylamino)-ethanol: there was produced 2-[2-Amino-4-(2-hydroxy-ethylamino)-quinazolin-7-yl]-3,N,N-trimethyl-benzamide trifluoroacetic acid salt as a white solid; (ES)⁺-HRMS m/e calcd for C₂₀H₂₃N₅O₂ (M+H)⁺ 366.1925, found 366.1929.

### Example 132

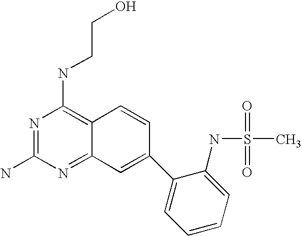

As described in Scheme 6, from N-(2-bromophenyl)-methanesulfonamide and 2-(2-Amino-7-tributylstannanyl-quinazolin-4-ylamino)-ethanol: there was produced N-{2-[2-Amino-4-(2-hydroxy-ethylamino)-quinazolin-7-yl]-phenyl}-methanesulfonamide trifluoroacetic acid salt as an off-white solid; (ES)⁺-HRMS m/e calcd for C₁₇H₁₉N₅O₃S (M+H)⁺ 374.1282, found 374.1284.

### Example 133

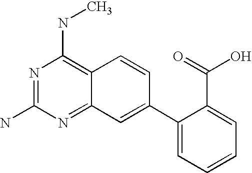

In a 25 ml flask, 2-(2-amino-4-methylamino-quinazolin-7-yl)-benzaldehyde (example 65, 62.3 mg, 0.224 mmol) was dissolved in of acetonitrile (3 mL). Sodium chlorite (40.5 mg, 0.448 mmol) and water (3 mL) were added to above solution, followed by addition of acetic acid (15 μL, 0.224 mmol). The reaction was stirred at ambient temperature for 16 h. The reaction mixture was adjusted to pH=1-2 with 1 N HCl. Upon concentration of the mixture, the residue was purified by silica gel flash chromatography then reverse phase HPLC. 2-(2-Amino-4-methylamino-quinazolin-7-yl)-benzoic acid trifluoroacetic acid salt were obtained as a white solid; LRMS for C₁₆H₁₄N₄O₂ (M+H)⁺ at m/z=295.

### Example 134

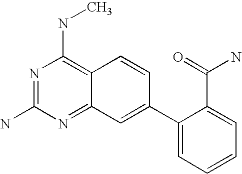

Isopropanol (5 mL) was placed in a 25 ml of round bottom flask, which was cooled in dry ice-acetonitrile. Ammonium gas was introduced into the cold solvent for 5 min. Sodium cyanide (0.13 g, 2.5 mmol) was added to above solution. The mixture was stirred for 5 min and 2-(N4-methyl-2,4-diamino-quinazolin-7-yl)-benzaldehyde (example 65, 53.4 mg, 0.19 mmol) was added, followed by addition of manganese (IV) oxide (0.38 g, 4.36 mmol). The mixture was then placed in an ice-water bath and warmed up to ambient temperature gradually. The reaction mixture was stirred for 16 h and filtered through a celite plug. Upon concentration of the filtrate, water was added to the residue. Thus formed solid was collected and further purified by reverse phase HPLC. There was obtained 2-(N4-methyl-2,4-diamino-quinazolin-7-yl)-benzamide as an off-white solid (44.9 mg, 80%); LRMS for C₁₆H₁₅N₅O (M+H)⁺ at m/z=294.

### Example 135

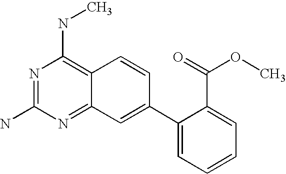

2-(2-Amino-4-methylamino-quinazolin-7-yl)-benzaldehyde (example 65, 0.13 g, 0.5 mmol) was dissolved in anhydrous methanol, followed by addition of manganese (IV) oxide (0.44 g, 5 mmol) and sodium cyanide (0.12 g, 2.5 mmol). The reaction mixture was stirred at ambient temperature for 16 h and was concentrated. To the residue, water and ethyl acetate were added. The organic layer was separated and concentrated. Upon separation on reverse phase HPLC, there was obtained 2-(2-amino-4-methylamino-quinazolin-7-yl)-benzoic acid methyl ester as a white solid (69.2 mg); LRMS for C₁₇H₁₆N₄O₂ (M+H)⁺ at m/z=309.

### Example 136

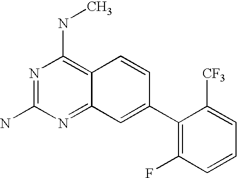

Preparation of N⁴-methyl-7-(4,4,5,5-tetramethyl-[1,3,2]dioxaborolan-2-yl)-quinazoline-2,4-diamine. 7-Bromo-N4-methyl-quinazoline-2,4-diamine (100 mg, 0.4 mmol), bis(pinacolato)diboron (151 mg, 0.6 mmol), [1,1-bis(diphenylphosphino)-ferrocene]dichloropalladium (9 mg, 0.012 mmol) and potassium acetate (118.2 mg, 1.2 mmol) were mixed in DMSO (4 mL). The resulted mixture was stirred and heated to 95° C. for 18 h. The reaction mixture was cooled and filtered through a short plug of mixture of silica gel, celite and sodium sulfate, washed with ethyl acetate and a mixture of acetonitrile and methanol and concentrated. Removal of DMSO via lyophilization, N⁴-methyl-7-(4,4,5,5-tetramethyl-[1,3,2]dioxaborolan-2-yl)-quinazoline-2,4-diamine was obtained and used in the following synthesis.

N⁴-Methyl-7-(4,4,5,5-tetramethyl-[1,3,2]dioxaborolan-2-yl)-quinazoline-2,4-diamine (2.2 mmol), 2-bromo-1-fluoro-3-trifluoromethylbenzene (694 mg, 2.8 mmol), tetrakis(triphenylphosphine)palladium (0) (253 mg, 0.22 mmol) and sodium carbonate (5 mL, 2 M aqueous solution) were suspended in a mixed solvents of DME (10 mL) and ethanol (10 mL) in a sealed pressure tube. The mixture was stirred and heated to 95° C. for 16 h. The reaction mixture was concentrated and dissolved in a mixture of THF and methanol (3:1). The solution was filtered through a plug of silica gel, celite and sodium sulfate. The filtrate was concentrated and purified by silica gel flash chromatography. The product was eluted with a mixed solvent of dichloromethane, methanol and ammonium hydroxide (95:5:0.5). The product was dissolved in dichloromethane (90%) and methanol (10%) and loaded onto a preparative thin layer chromatography (TLC) plate. The developing solvents were dichloromethane, methanol and ammonium hydroxide (90:10:1). The product was extracted out from the plate with THF (80%) and methanol (20%). ¹H NMR of the product showed the contamination with a stabilizer in THF. The preparative thin layer chromatography (TLC) procedure was repeated under same condition and the product was extracted out with dichloromethane (80%) and methanol (20%). There was obtained 7-(2-fluoro-6-trifluoromethyl-phenyl)-N4-methyl-quinazoline-2,4-diamine (120 mg) as a solid; LRMS for C₁₆H₁₂F₄N₄ (M+H)⁺ at m/z=337.

### Example 137

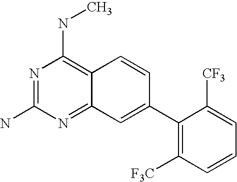

As described in Scheme 6, from N⁴-methyl-7-(4,4,5,5-tetramethyl-[1,3,2]dioxaborolan-2-yl)-quinazoline-2,4-diamine and 2,6-bis(trifluoromethyl)bromobenzene: 7-(2,6-bis-trifluoromethyl-phenyl)-N4-methyl-quinazoline-2,4-diamine trifluoroacetic acid salt as a light brown powder; LRMS for C₁₇H₁₂F₆N₄ (M+H)⁺ at m/z=387.

### Example 138

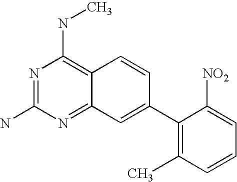

As described in Scheme 6, from N⁴-methyl-7-(4,4,5,5-tetramethyl-[1,3,2]dioxaborolan-2-yl)-quinazoline-2,4-diamine and 2-methyl-6-nitrobenzene: N4-methyl-7-(2-methyl-6-nitro-phenyl)-quinazoline-2,4-diamine trifluoroacetic acid salt as a light brown powder; LRMS for C₁₆H₁₅N₅O₂ (M+H)⁺ at m/z=310.

### Example 139

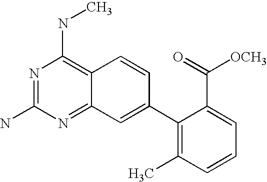

As described in Scheme 6, from bis(pinacolato)diboron and 3-methyl-2-bromobenzoic acid methyl ester, there was prepared 3-methyl-2-(4,4,5,5-tetramethyl-[1,3,2]dioxaborolan-2-yl)-benzoic acid and this compound was used in the following reaction.

As described in Scheme 6, from 7-bromo-N4-methyl-quinazoline-2,4-diamine and 3-methyl-2-(4,4,5,5-tetramethyl-[1,3,2]dioxaborolan-2-yl)-benzoic acid methyl ester: 2-(2-amino-4-methylamino-quinazolin-7-yl)-3-methyl-benzoic acid methyl ester as a light brown powder; LRMS for C₁₈H₁₈N₄O₂ (M+H)⁺ at m/z=323.

### Example 140

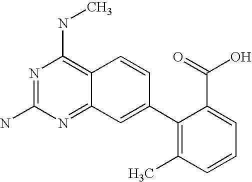

2-(2-Amino-4-methylamino-quinazolin-7-yl)-3-methyl-benzoic acid methyl ester (example 139, 4.5 mg, 0.013 mmol) was dissolved in a mixture of THF, methanol and water. Lithium hydroxide monohydrate (3 mg, 0.075 mmol) was added to above solution and the mixture was stirred at ambient temperature for 24 h. The solvent was removed under nitrogen and 1 M HCl aqueous solution (1 mL) was added. The white precipitate was collected by centrifugation and washed with water. 2-(2-Amino-4-methylamino-quinazolin-7-yl)-3-methyl-benzoic acid as a white powder; LRMS for C₁₇H₁₆N₄O₂ (M+H)⁺ at m/z=309.

### Example 141

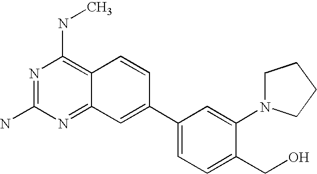

As described in Scheme 4, from 4-bromo-2-fluorobenzaldehyde and pyrrolidine, there was prepared 4-bromo-2-pyrrolidin-1-ylbenzaldehyde, LRMS for C₁₁H₁₂NOBr (M+H)⁺ at m/z=255.

As described in Scheme 6, from 4-bromo-2-pyrrolidin-1-ylbenzaldehyde and N4-methyl-7-tributylstannanyl-quinazoline-2,4-diamine, there was obtained 4-(2-amino-4-methylamino-quinazolin-7-yl)-2-pyrrolidin-1-ylbenzaldehyde; LRMS for C₂₀H₂₁N₅O (M+H)⁺ at m/z=348

4-(2-Amino-4-methylamino-quinazolin-7-yl)-2-pyrrolidin-1-ylbenzaldehyde (1.18 mmol) was dissolved in a solvent mixture of methanol and dichloromethane, followed by addition of sodium borohydrate (excess equivalent). The resulted mixture was stirred for 20 min and concentrated. After aqueous workup with water and ethyl acetate and reverse phase HPLC purification, there was obtained [4-(2-amino-4-methylamino-quinazolin-7-yl)-2-pyrrolidin-1-yl-phenyl]-methanol trifluoroacetic acid salt as a yellow solid; LRMS for C₂₀H₂₃N₅O (M+H)⁺ at m/z=350.

### Example 142

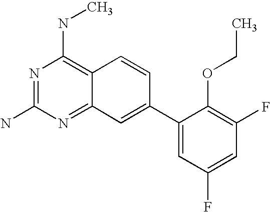

As described in Scheme 4, from 1-bromo-2,3,5-trifluorobenzene and ethanol, there was prepared 1-bromo-2-ethoxy-3,5-difluorobenzene; LRMS for C₈H₇BrOF2 (M+H)⁺ at m/z=238.

As described in Scheme 6, from 1-bromo-2-ethoxy-3,5-difluorobenzene and N4-methyl-7-tributylstannanyl-quinazoline-2,4-diamine: 7-(3,5-difluoro-2-ethoxy-phenyl)-N4-methyl-quinazoline-2,4-diamine as a yellow solid; LRMS for C₁₇H₁₆F₂N₄₀ (M+H)⁺ at m/z=331.

### Example 143

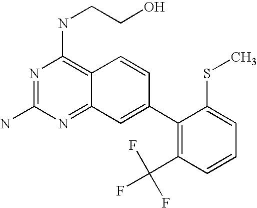

A mixture of 2-[2-Amino-7-bromo-quinazolin-4-ylamino]-ethanol (0.490 g, 1.731 mmol), tetrakis (triphenylphosphine) palladium(0) (0.026 g, 0.0130 mmol) and hexa-n-butylditin (2.0 mL, 3.958 mmol) in N,N-dimethylacetamide (2 mL) and toluene (12 mL) was refluxed for 16 hours. The reaction suspension was passed through a celite pad and the resulting solution was added to water (10 mL) and extracted (3×100 mL) with methylene chloride:methanol:ammonium hydroxide (9:1:0.1). The combined organic layers were dried over anhydrous magnesium sulfate, filtered and concentrated. Flash column chromatography of the crude product was carried out with methylene chloride:methanol:ammonium hydroxide (90:10:1) to give 2-(2-Amino-7-tributylstannanyl-quinazolin-4-ylamino)-ethanol (0.237 g, 27.8%) as a light yellow oil. EI-HRMS m/e calcd for C₂₂H₃₈N₄OSn (M+H⁺) 495.2146, found 495.2149

The coupling reaction can be carried out by a conventional aryl or heteroaromatic coupling partner utilizing Stille coupling. e.g. Stille et al., Angew. Chem. Int. Ed. Engl., 1986, 25, 508.

Typical conditions used to carry out the Stille reaction include the use of an organostannane as the coupling partner, palladium catalyst (2-20 mole %) such as tetrakis(triphenylphosphine)-palladium (0) or [1,1′bis(diphenylphosphino)-ferrocene]dichloro-palladium(II), a salt such as potassium fluoride or lithium chloride, in a suitable anhydrous solvent such as THF or DMF or ethylene glycol for at temperatures ranging from 25° C. to 125° C. for 2-18 hr

From 2-(2-Amino-7-tributylstannanyl-quinazolin-4-ylamino)-ethanol and 1-methylsulfanyl-2-bromo-3-trifluoromethyl-benzene prepared according to the general procedure detailed in Scheme 5: 2-[2-Amino-7-(2-methylsulfanyl-6-trifluoromethyl-phenyl)-quinazolin-4-ylamino]-ethanol as white solid; EI-HRMS m/e calcd for C₁₈H₁₇F₃N₄OS (M+H⁺) 395.1148, found 395.1152

### Example 144

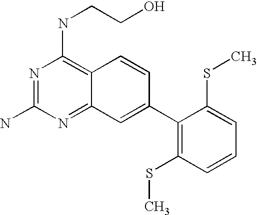

From 2-(2-Amino-7-tributylstannanyl-quinazolin-4-ylamino)-ethanol and 2,6-bis-methylsulfanyl-phenyl iodide prepared according to the general procedure detailed in Scheme 5 yielded 2-[2-Amino-7-(2,6-bis-methylsulfanyl-phenyl)-quinazolin-4-ylamino]-ethanol as white solid; EI-HRMS m/e calcd for C₁₈H₂₀N₄OS₂ (M+H⁺) 373.1152, found 373.1155

### Example 145

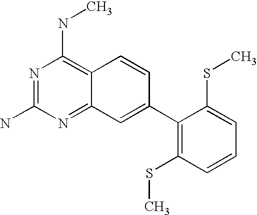

From N4-methyl-7-tributylstannanyl-quinazoline-2,4-diamine and 2,6-Bis-methylsulfanyl-phenyl iodide prepared according to the general procedure detailed in Scheme 5 there was produced: 7-(2,6-Bis-methylsulfanyl-phenyl)-N4-methyl-quinazoline-2,4-diamine as an off-white solid; EI-HRMS m/e calcd for C₁₇H₁₈N₄S2 (M+H⁺) 343.1046, found 343.1048

### Example 146

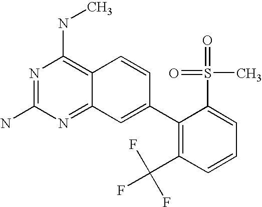

From oxidation of any conventional methods e.g. For general review of oxidation of thioethers to sulphoxide and sulphones, see Smith, M.; March, J.; Advanced Organic Chemistry, Wiley-Interscience: NY, 2001, pp. 1541-1542 and the references cited in it. Appropriate methods described in above reference of oxidation of an alkyl thio substituent to the corresponding sulfone group can be utilized to effect this conversion of N4-methyl-7-(2-methylsulfanyl-6-trifluoromethyl-phenyl)-quinazoline-2,4-diamine (example 86) yielding 7-(2-Methanesulfonyl-6-trifluoromethyl-phenyl)-N4-methyl-quinazoline-2,4-diamine as white solid; EI-HRMS m/e calcd for C₁₇H₁₅F₃N₄O₂S₂ (M+H⁺) 397.0941, found 397.0944

### Example 147

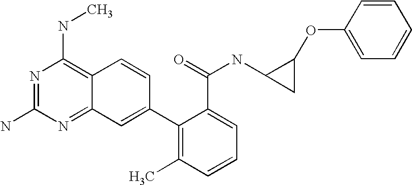

From the coupling reaction as described in Scheme 6 of 2-iodo-3-methylbenzoic acid and 2-phenoxycyclopropylamine (the procedure described by Jacob Finkelstein et al (J. Med. Chem., 8, 1965, 432-439), there was produced 2-iodo-3-methyl-N-(2-phenoxy-cyclopropyl)benamide.

As described in Scheme 7, from 2-iodo-3-methyl-N-(2-phenoxy-cyclopropyl)benamide and N4-methyl-7-tributylstannanyl-quinazoline-2,4-diamine: 2-(2-amino-4-methylamino-quinazolin-7-yl)-3-methyl-N-(2-phenoxy-cyclopropyl)-benzamide as a solid; LRMS for C₂₆H₂₅N₅O₂ (M+H)⁺ at m/z=440.

### Example 148

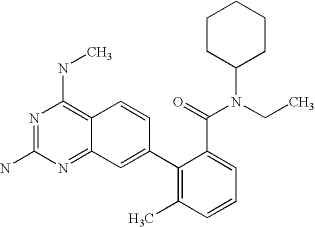

From the coupling reaction as described in Scheme 6 of 2-iodo-3-methylbenzoic acid and N-ethylcyclohexyllamine, there was produced N-cyclohexyl-N-ethyl-2-iodo-3-methyl-benamide. As described in Scheme 7, from N-cyclohexyl-N-ethyl-2-iodo-3-methyl-benamide and N4-methyl-7-tributylstannanyl-quinazoline-2,4-diamine: 2-(2-Amino-4-methylamino-quinazolin-7-yl)-N-cyclohexyl-N-ethyl-3-methylbenz-amide was yielded as a solid; LRMS for C₂₅H₃₁N₅O (M+H)⁺ at m/z=418.

### Example 149

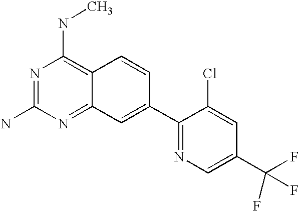

As described in Scheme 7, from 2-bromo-3-chloro-5-trifluoromethyl pyridine and N4-methyl-7-tributylstannanyl-quinazoline-2,4-diamine: 7-(3-Chloro-5-trifluoromethyl-pyridin-2-yl)-N4-methyl-quinazoline-2,4-diamine as a solid; LRMS for C₁₅H₁₁ClF₃N₅ (M+H)⁺ at m/z=354.

### Example 150

### In Vitro Inhibition of PTP1B

### Enzymes

Human PTP1B (1-321) was cloned from a human cDNA library using conventional molecular biology techniques. The cDNA sequence was identical to the published human PTP1B sequence (Accession number M33689). The protein was expressed and purified from E. coli as described by Barford D. et. al J. Mol Biol (1994) 239, 726-730.

### PTPase Assays

The measurement of PTPase activity was carried out using one of two methods:

The first method for the measurement of PTP1B inhibitory activity a tyrosine phosphorylated peptide based on the amino acid sequence of insulin receptor tyrosine autophosphorylation site 1146 (TRDI(pY)E) was used as substrate. The reaction conditions were as follows:

PTP1B (0.5-2 nM) was incubated with compound for 15 min in buffer containing 37.5 mM Bis-Tris buffer pH 6.2, 140 mMNaCl, 0.05% BSA and 2 mM DTT. The reaction was started by the addition of 50 μM substrate. After 20 min at room temperature (22-25° C.), the reaction was stopped with KOH and the amount of free phosphate measured using Malachite Green as previously described (Harder et al. 1994 Biochem J. 298; 395).

The second method was used for the measurement of general PTPase inhibitory activity across a panel of PTPases the substrate (6,8-difluoro-4-methylumbelliferyl phosphate (DiFMUP; from Molecular Probes) was used at the Km for each enzyme. The buffer conditions were identical as in the Malachite Green assay. The reaction was stopped with KOH. In this case the dephosphoryated product becomes fluorescent and the fluorescense read (Excitiation: 360 mM/Emmission: 460 nM).

For kinetic experiments, the same buffer conditions were used except that the reaction was started using enzyme and the reaction stopped after 10 minutes.

The IC50 values (in μM) for the PTP1B inhibitory activity of the compounds in the present application are in the range of 1.09 μM to 91.79 μM. The most preferred compounds show an IC50 of <30.0 μM.

### Example 151

### Glucose Uptake Assay

The day before the assay the SKMC media was changed to high glucose DMEM, 25 mM Hepes, pH 7.0 and 2% Charcoal/dextran treated FBS for 19 hours.

On the morning of the assay, cells were starved for max. 2 hours in low glucose (5.5 mM glucose) DMEM, 25 mM Hepes, pH 7.0 and 0.5% BSA. The starvation medium was removed and replaced with test medium (150 mMNaCl, 25 mM Hepes, pH 7.0) containing either 1% DMSO, or test compound diluted in DMSO or Porcine Insulin to a final concentrations of 1, 0.1, 0.05, 0.01 and 0.01 μM. Each assay point was performed in triplicate. The cells were incubated for 45 min at 37° C. 10 μM Cytochalasin B (CB) was added to appropriate wells to stop the active glucose transport (i.e., GLUT 1 & 4). At this point 2-Deoxy-D(U-¹⁴C)glucose (Amersham, Code CFB195, 200 uCi/ml) was added to all wells to a final concentration of 0.8 μCi/ml. The cells were incubated for an additional 45 minutes at 37° C. in an incubator. Cells were then very gently washed for three times in PBS (RT). The cells were then lysed with the addition of 0.05% NaOH solution for 20 min at RT. The lysate was transferred to a scintillation vial containing 5 ml of scintillation fluid and counted in a Beckman LS6500 Scintillation counter. Analysis of results: The counts obtained with CB (passive glucose transport values) were subtracted from every value obtained with PI (or compounds) in order to evaluate only active glucose transport. Fold increase was calculated by dividing values in the presence of PI (or compounds) by the value obtained in the presence of DMSO (control). Compounds were considered to be active when they increase glucose uptake at least 25% of the Porcine Insulin response at 0.05 μM.

In Vivo Inhibition of PTP1B: Effects of Compounds on Blood Glucose Levels in Mouse Model

To measure the anti-diabetic effect compounds were tested in well established rodent in vivo models of type 2 diabetes and obesity.

### Example 152

### Mouse Models

Diet Induced Obese (DIO) Mouse Model: A majority of male C57BL/6J mice fed a diet consisting of 35.5% fat for 3 months develop obesity, hyperinsulinemia and hyperglycemia. DIO mice are probably a better model for human type-2 diabetes than are genetic mutations with multiple neuroendocrine abnormalities. Furthermore, the DIO mice probably develop type-2 diabetes in a manner similar to most cases of type-2 diabetes in humans, e.g. only those predisposed individuals who become obese after access to a diabetogenic diet.

B6.C-m Lep^{db}/++/J: Mice homozygous for the diabetes spontaneous mutation (Lepr^{db}) become identifiably obese around 3 to 4 weeks of age. Elevations of plasma insulin begin at 10 to 14 days and of blood sugar at 4 to 8 weeks. Homozygous mutant mice are polyphagic, polydipsic, and polyuric. The course of the disease is markedly influenced by genetic background. A number of features are observed on the C57BLKS background, including an uncontrolled rise in blood sugar, severe depletion of the insulin-producing beta-cells of the pancreatic islets, and death by 10 months of age. Exogenous insulin fails to control blood glucose levels and gluconeogenic enzyme activity increases. Peripheral neuropathy and myocardial disease are seen in C57BLKS Lepr^{db} homozygotes.

B6.V-Lep^{ob}/J: Mice homozygous for the obese spontaneous mutation, (Lep^{ob} commonly referred to as ob or ob/ob), are first recognizable at about 4 weeks of age. Homozygous mutant mice increase in weight rapidly and may reach three times the normal weight of wildtype controls. In addition to obesity, mutant mice exhibit hyperphagia, a diabetes-like syndrome of hyperglycemia, glucose intolerance, elevated plasma insulin, subfertility, impaired wound healing, and an increase in hormone production from both pituitary and adrenal glands. They are also hypometabolic and hypothermic. The obesity is characterized by an increase in both number and size of adipocytes. Although hyperphagia contributes to the obesity, homozygotes gain excess weight and deposit excess fat even when restricted to a diet sufficient for normal weight maintenance in lean mice. Hyperinsulinemia does not develop until after the increase body weight and is probably the result of it. Homozygotes do have an abnormally low threshold for stimulation of pancreatic islet insulin secretion even in very young preobese animals. Female homozygotes exhibit decreased uterine and ovarian weights, decreased ovarian hormone production and hypercytolipidemia in follicular granulosa and endometrial epithelial tissue layers (Garris et al., 2004).

### Mouse Criteria:

DIO Mouse Model: Mice used in these studies were at least 18 weeks of age and maintained on a high fat diet (BioServ F3282) for at least 12 weeks, The mice were weighed on the day prior to the study and sorted into treatment groups. Because of the variability in body weights, the DIO mice having the most extreme (i.e. highest or lowest) body weights were excluded.

B6.C-m Lep^{db}/++/J: Mice used in these studies were at least 9 weeks of age and maintained on Purina Lab Diet 5008 starting at 6 weeks of age. Two to three days prior to the study blood glucose levels of the mice were determined following a two hour fast. The mice were sorted into treatment groups. Because of the variability in blood glucose levels, the mice having the most extreme (i.e. highest or lowest) blood glucose levels were excluded. We tried to achieve an average blood glucose level between 160-190 mg/dl.

B6.V-Lep^{ob}/J: Mice used in these studies were at least 7 weeks of age and maintained on Purina Lab Diet 5001. Two to three days prior to the study blood glucose levels of the mice were determined following a two hour fast. The mice were sorted into treatment groups. Because of the variability in blood glucose levels, the mice having the most extreme (i.e. highest or lowest) blood glucose levels were excluded. In some instances mice were sorted based on body weights, the ob/ob mice having the most extreme (i.e. highest or lowest) body weights were excluded.

### Experimental Parameters:

Oral Glucose Tolerance Test (OGTT): Mice were placed into individual cages and fasted for 15 hours. After 15 hours the mice were treated orally by gavage with vehicle or compound using a dose volume of 5 ml/kg. An oral glucose challenge (1-2 g/kg) was administered four hours following treatment. Blood was collected from the tail vein into a 20 ul heparinized microhematocrit tube immediately prior to dosing with vehicle or compound, immediately prior to the OGTT and 0.5, 1, 1.5, 2 and sometimes up to 4 hours following the OGTT. The blood was transferred immediately to a microfuge tube. Blood glucose was measured with the YSI 2700 Select Glucose Analyzer. In some instances mice were fasted for only 2 hours prior to dosing with vehicle or compound and the OGTT was administered 4 hours post dose.

Acute Efficacy Study: Mice were placed into individual cages and fasted for 2 hours. After 2 hours the mice were treated orally by gavage with vehicle or compound using a dose volume of 5 ml/kg. Blood was collected from the tail vein into a 20 ul heparinized microhematocrit tube immediately prior to dosing with vehicle or compound and 2, 4, 6 and 8 hours following treatment. The blood was transferred immediately to a microfuge tube. Blood glucose was measured with the YSI 2700 Select Glucose Analyzer

Mice that have type 2 diabetes were generated by maintaining them on a high fat diet for 4-6 months (Diabetes vol. 37 September 1988). Male C57BL/6J mice (age 3-4 weeks) were placed on high fat diet for 4-6 months. At this time they were hyperglycemic and hyperinsulinemic and weighed 40-50 g. DIO mice (n=10) were weighed and fasted for a two hour period prior to oral treatment. Immediately prior to dosing a pre-dose blood glucose reading was taken by snipping off a portion of the tail and collecting blood from the tail vein. Mice were treated either with a single dose of compound (acute) or once a day for 5 days (sub-chronic). For the acute studies, glucose was generally measured at 2 h, 4 h, 6 h, 8 h post treatment. Compounds were considered active if the compounds demonstrated AUC (Area under the curve) showed a statistically significant (p≦0.05) glucose lowering (>15%) compared to the vehicle treated animals.

For sub-chronic (5 day) studies mice were dosed once a day by gavage as described above. On day five, glucose was measured prior to dosing (0 time) and 2 hours after dosing. Insulin and triglycerides were measured at 2 hour post dose. Compounds were considered active if the compounds demonstrated AUC (Area under the curve) showed a statistically significant (p≦0.05) glucose, insulin and triglyceride lowering compared to the vehicle treated animals.

The following are examples of compounds which tested positively in the in vivo mouse models and gave the indicated IC50 activities in accordance with the procedures described in Example 150:

| Example | IC50 (μM) |
|---|---|
| 6 | 3.15 |
| 8 | 0.79 |
| 9 | 13.98 |
| 14 | 2.48 |
| 21 | 3.50 |
| 24 | 1.11 |
| 36 | 3.17 |
| 41 | 18.97 |
| 43 | 6.02 |
| 57 | 3.34 |
| 58 | 1.01 |
| 69 | 4.04 |
| 71 | 2.78 |
| 72 | 1.44 |
| 75 | 4.18 |
| 82 | 1.39 |
| 89 | 1.46 |
| 120 | 1.60 |
| 125 | 1.89 |
| 126 | 1.94 |

What is claimed is:

1. A compound of the formula: 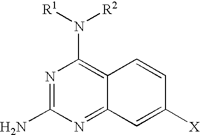
wherein X is a group X-1 of the formula: 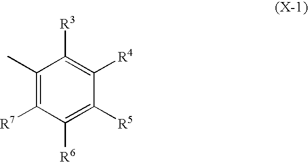
or X is a group X-2 of the formula: 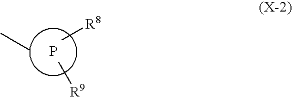
wherein
R¹ and R² are each independently selected from the group consisting of hydrogen, lower alkyl, alkoxy lower alkyl, and hydroxy lower alkyl, except that R¹ and R² may not both be hydrogen; R³, R⁴, R⁶ and R⁷ are each independently selected from the group consisting of hydrogen, lower alkyl, substituted lower alkyl, lower alkoxy, substituted lower alkoxy, hydroxy, halogen, lower alkylthio, lower alkylsulfinyl, lower alkylsulfonyl, aminosulfonyl, cyano, nitro, carbamoyl, lower alkylcarbamoyl, lower alkanoyl, aroyl, aryl, aryloxy, aryl lower alkoxy, aryl lower alkenyl, aryl lower alkynyl, lower alkenyl, lower alkynyl, lower alkylamino, substituted lower alkylamino, lower alkanoylamino, sulfonylamino, cycloalkyl, heterocycloalkyl, heterocyclyloxy, heterocyclylcarbonyl, carboxyl, lower alkoxy carbonyl, and a substituent of the formula: 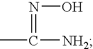
R⁵ is selected from the group consisting of hydrogen, lower alkyl, lower alkoxy, alkoxy lower alkyl, alkoxy lower alkoxy, hydroxy lower alkyl, hydroxy, hydroxyalkoxy, halogen, lower alkylthio, lower alkylsulfinyl, lower alkylsulfonyl, perfluoro lower alkyl, lower alkanoyl, aroyl, aryl alkynyl, lower alkynyl and lower alkanoylamino; \textcircled{P} is a 5 or 6 membered heteroaromatic ring containing from 1 to 2 hetero atoms selected from the group consisting of oxygen, sulfur and nitrogen;
R⁸ and R⁹ are each independently hydrogen, lower alkyl, lower alkoxy, perfluoro lower alkyl, halogen, aryl lower alkyl, aryl, or aryl lower alkoxy;
or the pharmaceutically acceptable salt thereof.

2. The compound of claim 1 of the formula: 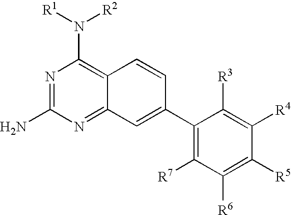
or the pharmaceutically acceptable salts thereof.

3. The compound of claim 2 wherein R⁴, R⁵ and R⁶ are each independently hydrogen, halogen, lower alkyl, lower alkoxy, hydroxy, hydroxy lower alkyl, lower alkylthio, lower alkyl sulfonyl or perfluoro lower alkyl.

4. The compound of claim 2 wherein only R⁶ is hydrogen.

5. The compound of claim 2 wherein R⁶ and only one of R⁴ or R⁶ is hydrogen.

6. The compound of claim 2 where R⁴, R⁵ and R⁶ are hydrogen.

7. The compound of claim 5 wherein the R⁴ or R⁶ which is substituted is substituted with halogen, lower alkyl, lower alkoxy, hydroxy, hydroxy lower alkyl, lower alkylthio, lower alkyl sulfonyl or perfluoro lower alkyl.

8. The compound of claim 2 wherein R³ and R⁷ are each independently hydrogen, halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

9. The compound of claim 3 wherein R³ and R⁷ are each independently hydrogen, halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

10. The compound of claim 4 wherein R³ and R⁷ are each independently hydrogen, halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

11. The compound of claim 5 wherein R³ and R⁷ are each independently hydrogen, halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

12. The compound of claim 6 wherein R³ and R⁷ are each independently hydrogen, halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

13. The compound of claim 7 wherein R³ and R⁷ are each independently hydrogen, halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

14. The compound of claim 5 wherein R³ and R⁷ are each independently halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

15. The compound of claim 6 wherein R³ and R⁷ are each independently halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

16. The compound of claim 7 wherein R³ and R⁷ are each independently halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

17. The compound of claim 2 wherein R¹ or R² is hydrogen.

18. The compound of claim 17 wherein the R¹ or R² which is substituted is substituted with C1-4 alkyl or hydroxy C1-3 alkyl.

19. The compound of claim 17 wherein R⁴, R⁵ and R⁶ are each independently hydrogen, halogen, lower alkyl, lower alkoxy, hydroxy, hydroxy lower alkyl, lower alkylthio, lower alkyl sulfonyl or perfluoro lower alkyl.

20. The compound of claim 17 wherein only R⁶ is hydrogen.

21. The compound of claim 17 wherein R⁶ and only one of R⁴ or R⁶ is hydrogen.

22. The compound of claim 21 wherein the R⁴ or R⁶ which is substituted is substituted with halogen, lower alkyl, lower alkoxy, hydroxy, hydroxy lower alkyl, lower alkylthio, lower alkyl sulfonyl or perfluoro lower alkyl.

23. The compound of claim 17 wherein R⁴, R⁵ and R⁶ are hydrogen.

24. The compound according to claim 23 wherein the R¹ or R² which is substituted is substituted with C1-4 alkyl or hydroxy C1-3 alkyl.

25. The compound of claim 17 wherein R³ and R⁷ are each independently hydrogen, halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

26. The compound of claim 19 wherein R³ and R⁷ are each independently hydrogen, halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

27. The compound of claim 20 wherein R³ and R⁷ are each independently hydrogen, halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

28. The compound of claim 21 wherein R³ and R⁷ are each independently hydrogen, halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

29. The compound of claim 22 wherein R³ and R⁷ are each independently hydrogen, halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

30. The compound of claim 23 wherein R³ and R⁷ are each independently hydrogen, halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

31. The compound of claim 30 wherein the R¹ or R² which is substituted is substituted with C1-4 alkyl or hydroxy C1-3 alkyl.

32. The compound of claim 21 wherein R³ and R⁷ are each independently halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

33. The compound of claim 22 wherein R³ and R⁷ are each independently halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

34. The compound of claim 23 wherein R³ and R⁷ are each independently halogen, lower alkyl, lower alkoxy, alkoxy lower alkoxy, nitro, hydroxy, hydroxy lower alkoxy, hydroxy lower alkyl, lower alkylthio, lower alkyl sulfonyl, and perfluoro lower alkyl.

35. The compound of claim 34 wherein the R¹ or R² which is substituted is substituted with C1-4 alkyl or hydroxy C1-3 alkyl.

36. The compound of claim 23 wherein R³ and R⁷ are chlorine, fluorine, trifluoromethyl, C1-4 alkyl, C1-3 alkylthio, C1-3 alkylsulfonyl, C1-3 alkoxy, C1-3 alkoxy substituted with a group selected from hydroxy, methoxy and ethoxy

37. The compound of claim 36 wherein the R¹ or R² which is substituted is substituted with C1-4 alkyl or hydroxy C1-3 alkyl.

38. The compound of claim 1 of the formula: 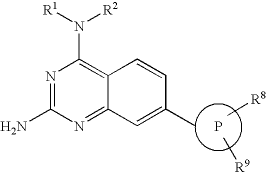
or the pharmaceutically acceptable salts thereof.

39. The compound of claim 38 wherein R¹ or R² is hydrogen.

40. The compound of claim 38 wherein R⁸ and R⁹ are each independently lower alkyl, lower alkoxy, perfluoro lower alkyl or halogen.

41. The compound of claim 39 wherein R⁸ and R⁹ are each independently lower alkyl, lower alkoxy, perfluoro lower alkyl or halogen.

42. The compound of claim 39 wherein the R¹ or R² which is substituted is substituted with C1-4 alkyl or hydroxy C1-3 alkyl.

43. The compound of claim 41 wherein the R¹ or R² which is substituted is substituted with C1-4 alkyl or hydroxy C1-3 alkyl.

44. A pharmaceutical composition comprising a pharmaceutically effective amount of a compound of claim 2 and a pharmaceutically acceptable carrier and/or diluent.

45. A pharmaceutical composition comprising a pharmaceutically effective amount of a compound of claim 38 and a pharmaceutically acceptable carrier and/or diluent.

46. A method for the treatment of diabetes comprising administering to a patient in need thereof a therapeutically effective amount of a composition of claim 44.

47. A method for the treatment of diabetes comprising administering to a patient in need thereof a therapeutically effective amount of a composition of claim 45.

48. The compound of claim 1 selected from the group consisting of:
7-(2,5-dimethyl-phenyl)-quinazoline-2,4-diamine,
N4-Methyl-7-(2-trifluoromethyl-phenyl)-quinazoline-2,4-diamine,
N4,N4-Dimethyl-7-(2-trifluoromethyl-phenyl)-quinazoline-2,4-diamine,
N4-Methyl-7-thiophen-2-yl-quinazoline-2,4-diamine,
N4,N4-Dimethyl-7-thiophen-2-yl-quinazoline-2,4-diamine,
N4-Methyl-7-o-tolyl-quinazoline-2,4-diamine,
N4,N4-Dimethyl-7-o-tolyl-quinazoline-2,4-diamine,
7-(2,6-Dimethyl-phenyl)-N4-methyl-quinazoline-2,4-diamine,
7-(2,6-Dimethyl-phenyl)-N4,N4-dimethyl-quinazoline-2,4-diamine; and
N4-Ethyl-7-o-tolyl-quinazoline-2,4-diamine.

49. The compound of claim 1 selected from the group consisting of:
N4,N4-Diethyl-7-o-tolyl-quinazoline-2,4-diamine,
N4-Propyl-7-o-tolyl-quinazoline-2,4-diamine,
N4,N4-Dipropyl-7-o-tolyl-quinazoline-2,4-diamine,
7-(2,6-Dimethyl-phenyl)-N4-ethyl-quinazoline-2,4-diamine,
7-(2,6-Dimethyl-phenyl)-N4,N4-diethyl-quinazoline-2,4-diamine,
7-(2,6-Dimethyl-phenyl)-N4-propyl-quinazoline-2,4-diamine,
7-(2,6-Dimethyl-phenyl)-N4,N4-dipropyl-quinazoline-2,4-diamine,
N4,N4-Dimethyl-7-(2-phenoxy-phenyl)-quinazoline-2,4-diamine,
7-(2,6-Difluoro-phenyl)-N4,N4-dimethyl-quinazoline-2,4-diamine, and
7-(2-Ethyl-phenyl)-N4-methyl-quinazoline-2,4-diamine.

50. The compound of claim 1 selected from the group consisting of:
7-(2,6-Dimethoxy-phenyl)-N4-methyl-quinazoline-2,4-diamine,
N4-Methyl-7-(1,3,5-trimethyl-1H-pyrazol-4-yl)-quinazoline-2,4-diamine,
7-(2,6-Difluoro-phenyl)-N4-methyl-quinazoline-2,4-diamine,
7-(2,6-Dichloro-phenyl)-N4-methyl-quinazoline-2,4-diamine,
7-(2-Isopropyl-phenyl)-N4-methyl-quinazoline-2,4-diamine,
7-(2-Isopropyl-phenyl)-N4,N4-dimethyl-quinazoline-2,4-diamine,
7-(2-Ethyl-phenyl)-N4,N4-dimethyl-quinazoline-2,4-diamine,
7-(2-bromo-phenyl)-N4-methyl-quinazoline-2,4-diamine,
N4-Methyl-7-phenyl-quinazoline-2,4-diamine, and
7-(2′-Bromo-biphenyl-2-yl)-4-methyl-quinazoline-2,4-diamine.

51. The compound of claim 1 selected from the group consisting of:
N4-methyl-7-o-tolyl-quinazoline-2,4-diamine,
7-(2-Methoxy-phenyl)-N4-methyl-quinazoline-2,4-diamine,
2-[2-Amino-7-(2-ethylsulfanyl-phenyl)-quinazolin-4-ylamino]-ethanol,
N4,N4-Dimethyl-7-(2,3,5,6-tetramethyl-phenyl)-quinazoline-2,4-diamine,
N4-Methyl-7-(2-phenoxy-phenyl)-quinazoline-2,4-diamine,
2-[2-Amino-7-(2,6-dimethyl-phenyl)-quinazolin-4-ylamino]-ethanol,
2-[2-Amino-7-(2-phenoxy-phenyl)-quinazolin-4-ylamino]-ethanol,
2-[2-Amino-7-(2,6-dichloro-phenyl)-quinazolin-4-ylamino]-ethanol,
2-[2-Amino-7-(2,6-difluoro-phenyl)-quinazolin-4-ylamino]-ethanol, and
2-[2-Amino-7-(2,5-difluoro-phenyl)-quinazolin-4-ylamino]-ethanol.

52. The compound of claim 1 selected from the group consisting of:
2-[2-Amino-7-(2-fluoro-phenyl)-quinazolin-4-ylamino]-ethanol,
2-[2-Amino-7-(2,3-dichloro-phenyl)-quinazolin-4-ylamino]-ethanol,
2-(2-Amino-7-o-tolyl-quinazolin-4-ylamino)-ethanol,
7-(2-Fluoro-6-methoxy-phenyl)-N4-methyl-quinazoline-2,4-diamine,
1-(2-Amino-7-o-tolyl-quinazolin-4-ylamino)-propan-2-ol,
3-(2-Amino-7-o-tolyl-quinazolin-4-ylamino)-propan-1-ol,
2-(2-Amino-7-o-tolyl-quinazolin-4-ylamino)-propan-1-ol,
N4-(2-Amino-ethyl)-7-o-tolyl-quinazoline-2,4-diamine,
[3-(2-Amino-4-methylamino-quinazolin-7-yl)-phenyl]-methanol, and
7-(5-Isopropyl-2-methoxy-phenyl)-N4-methyl-quinazoline-2,4-diamine.

53. The compound of claim 1 selected from the group consisting of:
7-(3-Isopropyl-phenyl)-N4-methyl-quinazoline-2,4-diamine,
7-(3,5-Dichloro-phenyl)-N4-methyl-quinazoline-2,4-diamine,
7-(2-Chloro-phenyl)-N4-methyl-quinazoline-2,4-diamine,
7-(2,5-Dichloro-phenyl)-N4-methyl-quinazoline-2,4-diamine,
7-Biphenyl-3-yl-N4-methyl-quinazoline-2,4-diamine,
7-(2,3-Dichloro-phenyl)-N4-methyl-quinazoline-2,4-diamine,
2-[2-Amino-7-(2-trifluoromethyl-phenyl)-quinazolin-4-ylamino]-ethanol,
N4-Methyl-7-(2-methylsulfanyl-phenyl)-quinazoline-2,4-diamine,
7-Biphenyl-2-yl-N4-methyl-quinazoline-2,4-diamine, and
N4-Methyl-7-(3-methylsulfanyl-phenyl)-quinazoline-2,4-diamine.

54. The compound of claim 1 selected from the group consisting of:
N4-Methyl-7-(4-methylsulfanyl-phenyl)-quinazoline-2,4-diamine,
7-(3-Ethoxy-phenyl)-N4-methyl-quinazoline-2,4-diamine,
3-(2-Amino-4-methylamino-quinazolin-7-yl)-benzonitrile,
N-[3-(2-Amino-4-methylamino-quinazolin-7-yl)-phenyl]-acetamide,
2-(2-amino-4-methylamino-quinazolin-7-yl)-benzaldehyde,
7-(3,5-Dimethyl-isoxazol-4-yl)-N4-methyl-quinazoline-2,4-diamine,
N-[3-(2-Amino-4-methylamino-quinazolin-7-yl)-phenyl]-methanesulfonamide,
7-(4-Ethylsulfanyl-phenyl)-N4-methyl-quinazoline-2,4-diamine,
7-(4-Fluoro-2-methyl-phenyl)-N4-methyl-quinazoline-2,4-diamine, and
2-(2-Amino-4-methylamino-quinazolin-7-yl)-benzonitrile.

55. The compound of claim 1 selected from the group consisting of:
7-(2-Methanesulfinyl-phenyl)-N4-methyl-quinazoline-2,4-diamine,
7-(2-Methanesulfonyl-phenyl)-N4-methyl-quinazoline-2,4-diamine,
2,6-dimethyl-phenyl)-quinazolin-4-ylamino]-propan-2-ol,
1-[2-Amino-7-(2,6-dichloro-phenyl)-quinazolin-4-ylamino]-propan-2-ol,
2-[2-Amino-7-(2-methylsulfanyl-phenyl)-quinazolin-4-ylamino]-ethanol,
2-[2-Amino-4-(2-hydroxy-ethylamino)-quinazolin-7-yl]-benzonitrile,
2-[2-Amino-7-(2-methanesulfonyl-phenyl)-quinazolin-4-ylamino]-ethanol,
2-[2-Amino-7-(2-methanesulfinyl-phenyl)-quinazolin-4-ylamino]-ethanol,
2-(2-Amino-4-methylamino-quinazolin-7-yl)-N-hydroxy-benzamidine, and
N-[2-(2-Amino-4-methylamino-quinazolin-7-yl)-phenyl]-methanesulfonamide.

56. The compound of claim 1 selected from the group consisting of:
7-(2-Ethylsulfanyl-phenyl)-N4-methyl-quinazoline-2,4-diamine,
7-(2-Ethanesulfonyl-phenyl)-N4-methyl-quinazoline-2,4-diamine,
7-[2-(4-Benzyl-piperazin-1-yl)-6-fluoro-phenyl]-N4-methyl-quinazoline-2,4-diamine,
7-(2-Fluoro-6-pyrrolidin-1-yl-phenyl)-N4-methyl-quinazoline-2,4-diamine,
N4-Methyl-7-(2-pyrrolidin-1-yl-6-trifluoromethyl-phenyl)-quinazoline-2,4-diamine,
N4-methyl-7-(2-methylsulfanyl-6-trifluoromethyl-phenyl)-quinazoline-2,4-diamine,
[2-(2-amino-4-methylamino-quinazolin-7-yl)-3-methyl-phenyl]-methanol,
7-(2-Ethylsulfanyl-6-trifluoromethyl-phenyl)-N4-methyl-quinazoline-2,4-diamine, and
7-(2-Chloro-6-methyl-phenyl)-N4-methyl-quinazoline-2,4-diamine and
7-(2,6-Dichloro-phenyl)-N4-methyl-quinazoline-2,4-diamine.

57. The compound of claim 1 selected from the group consisting of:
7-(3,5-difluoro-2-pyrrolidin-1-yl-phenyl)-N4-methyl-quinazoline-2,4-diamine,
7-[2-(2-methoxy-ethoxy)-phenyl]-N4-methyl-quinazoline-2,4-diamine,
N4-methyl-7-[2-(2,2,6,6-tetramethyl-piperidin-4-yloxy)-phenyl]-quinazoline-2,4-diamine,
N4-Methyl-7-(2,4,6-trifluoro-phenyl)-quinazoline-2,4-diamine,
N4-Methyl-7-[2,4,6-tris-(2-methoxy-ethoxy)-phenyl]-quinazoline-2,4-diamine,
7-[4-chloro-2,3,5,6-tetrakis-(2-methoxy-ethoxy)-phenyl]-N4-methyl-quinazoline-2,4-diamine,
7-[4-chloro-2-(2-methoxy-ethoxy)-phenyl]-N4-methyl-quinazoline-2,4-diamine,
1-[2-(2-Amino-4-methylamino-quinazolin-7-yl)-5-chloro-phenyl]-piperidin-4-ol,
2-(2-amino-4-methylamino-quinazolin-7-yl)-3-methyl-benzamide, and
2-(2-amino-4-methylamino-quinazolin-7-yl)-3,N,N-trimethyl-benzamide.

58. The compound of claim 1 selected from the group consisting of:
2-(2-amino-4-methylamino-quinazolin-7-yl)-3,N-dimethyl-benzamide,
2-(2-amino-4-methylamino-quinazolin-7-yl)-N-ethyl-3-methyl-benzamide,
7-(4-chloro-2-ethoxy-phenyl)-N4-methyl-quinazoline-2,4-diamine,
7-(2-ethoxy-6-fluoro-4-methoxy-phenyl)-N4-methyl-quinazoline-2,4-diamine,
7-(2-ethoxy-4-trifluoromethyl-phenyl)-N4-methyl-quinazoline-2,4-diamine,
7-(2-ethoxy-6-trifluoromethyl-phenyl)-N4-methyl-quinazoline-2,4-diamine,
2-(2-Amino-4-methylamino-quinazolin-7-yl)-N,N-diethyl-3-methyl-benzamide,
1-[2-(2-Amino-4-methylamino-quinazolin-7-yl)-phenyl]-ethanone,
2-[2-(2-Amino-4-methylamino-quinazolin-7-yl)-phenyl]-ethanol, and
7-(2-fluoro-4,6-dimethoxy-phenyl)-N4-methyl-quinazoline-2,4-diamine.

59. The compound of claim 1 selected from the group consisting of:
7-[2-(2-methoxy-ethoxy)-4-trifluoromethyl-phenyl]-N4-methyl-quinazoline-2,4-diamine,
7-[6-fluoro-4-methoxy-2-(2-methoxy-ethoxy)-phenyl]-N4-methyl-quinazoline-2,4-diamine,
2-(2-amino-4-methylamino-quinazolin-7-yl)-3, N-dimethyl-N-propyl-benzamide,
2-[2-(2-amino-4-methylamino-quinazolin-7-yl)-3-trifluoromethyl-phenylamino]-ethanol,
[2-(2-amino-4-methylamino-quinazolin-7-yl)-phenyl]-phenyl-methanone,
N-[2-(2-Amino-4-methylamino-quinazolin-7-yl)-phenyl]-acetamide,
7-(2-difluoromethoxy-phenyl)-N4-methyl-quinazoline-2,4-diamine,
[2-(2-amino-4-methylamino-quinazolin-7-yl)-3-methyl-phenyl]-piperidin-1-yl-methanone,
2-[2-(2-amino-4-methylamino-quinazolin-7-yl)-3-trifluoromethyl-phenoxy]-ethanol, and
2-(2-aAmino-4-methylamino-quinazolin-7-yl)-N,N-dimethyl-benzenesulfonamide.

60. The compound of claim 1 selected from the group consisting of:
2-(2-amino-4-methylamino-quinazolin-7-yl)-benzenesulfonamide,
3-[2-(2-Amino-4-methylamino-quinazolin-7-yl)-3-trifluoromethyl-phenoxy]-propane-1,2-diol,
7-[2-(2-Methoxy-ethoxy)-6-trifluoromethyl-phenyl]-N4-methyl-quinazoline-2,4-diamine,
7-[5-fluoro-4-methoxy-3-(2-methoxy-ethoxy)-phenyl]-N4-methyl-quinazoline-2,4-diamine,
2-(2-Amino-4-methylamino-quinazolin-7-yl)-benzenesulfonamide,
N-methyl-2-(2-amino-4-methylamino-quinazolin-7-yl)-benzenesulfonamide,
7-[6-fluoro-2-(2-hydroxy-ethoxy)-phenyl]-N4-methyl-quinazoline-2,4-diamine,
7-[2-(2,2-dimethyl-[1,3]dioxolan-4-ylmethoxy)-6-fluoro-phenyl]-N4-methyl-quinazoline-2,4-diamine,
3-[2-(2-amino-4-methylamino-quinazolin-7-yl)-3-fluoro-phenoxy]-propane-1,2-diol, and
2-[2-(2-Amino-4-methylamino-quinazolin-7-yl)-5-fluoro-phenoxy]-ethanol.

61. The compound of claim 1 selected from the group consisting of:
2-[2-Amino-4-(2-hydroxy-ethylamino)-quinazolin-7-yl]-3,N,N-trimethyl-benzamide,
N-{2-[2-Amino-4-(2-hydroxy-ethylamino)-quinazolin-7-yl]-phenyl}-methanesulfonamide,
2-(2-Amino-4-methylamino-quinazolin-7-yl)-benzoic acid,
2-(N4-methyl-2,4-diamino-quinazolin-7-yl)-benzamide,
2-(2-amino-4-methylamino-quinazolin-7-yl)-benzoic acid methyl ester,
7-(2-fluoro-6-trifluoromethyl-phenyl)-N4-methyl-quinazoline-2,4-diamine,
7-(2,6-bis-trifluoromethyl-phenyl)-N4-methyl-quinazoline-2,4-diamine,
N4-methyl-7-(2-methyl-6-nitro-phenyl)-quinazoline-2,4-diamine,
2-(2-amino-4-methylamino-quinazolin-7-yl)-3-methyl-benzoic acid methyl ester, and
2-(2-Amino-4-methylamino-quinazolin-7-yl)-3-methyl-benzoic acid.

62. The compound of claim 1 selected from the group consisting of:
[4-(2-amino-4-methylamino-quinazolin-7-yl)-2-pyrrolidin-1-yl-phenyl]-methanol,
7-(3,5-difluoro-2-ethoxy-phenyl)-N4-methyl-quinazoline-2,4-diamine,
2-[2-Amino-7-(2-methylsulfanyl-6-trifluoromethyl-phenyl)-quinazolin-4-ylamino]-ethanol,
2-[2-Amino-7-(2,6-bis-methylsulfanyl-phenyl)-quinazolin-4-ylamino]-ethanol,
7-(2,6-Bis-methylsulfanyl-phenyl)-N4-methyl-quinazoline-2,4-diamine,
7-(2-Methanesulfonyl-6-trifluoromethyl-phenyl)-N4-methyl-quinazoline-2,4-diamine,
2-(2-amino-4-methylamino-quinazolin-7-yl)-3-methyl-N-(2-phenoxy-cyclopropyl)-benzamide,
2-(2-Amino-4-methylamino-quinazolin-7-yl)-N-cyclohexyl-N-ethyl-3-methylbenz-amide, and
7-(3-Chloro-5-trifluoromethyl-pyridin-2-yl)-N4-methyl-quinazoline-2,4-diamine.
